(19) United States
(12) Patent Application Publication
(10) Pub. No.: US 2026/0215072 A1
(43) Pub. Date: Jul. 23, 2026

(54) MIXED COMPOSITION AND ORGANIC ELECTROLUMINESCENT ELEMENT

(71) Applicant: NIPPON STEEL Chemical & Material Co., Ltd., Tokyo (JP)

(72) Inventors: Mitsuru SAKAI, Tokyo (JP); Satoshi UKIGAI, Tokyo (JP); Tetsuro YAMASHITA, Tokyo (JP); Yuji IKENAGA, Tokyo (JP)

(73) Assignee: NIPPON STEEL Chemical & Material Co., Ltd., Tokyo (JP)

(21) Appl. No.: 19/142,645

(22) PCT Filed: Feb. 8, 2024
(86) PCT No.: PCT/JP2024/004354

§ 371 Date: Jun. 24, 2025

(30) Foreign Application Priority Data
Feb. 16, 2023 (JP) 2023-022230

(51) Int. Cl. H10K 50/11; C07B 59/00; C07D 403/14; C09K 11/06; H10K 85/30; H10K 85/60; H10K 101/00

(52) U.S. Cl. CPC H10K 50/11; C07B 59/002; C07D 403/14; C09K 11/06; H10K 85/342; H10K 85/346; H10K 85/371; H10K 85/615; H10K 85/636; H10K 85/654; H10K 85/6572; H10K 85/6574; H10K 85/658; C07B 2200/05; C09K 2211/1029; H10K 2101/90

(57) ABSTRACT

To provide a practically useful organic EL device having a low driving voltage and also having a high efficiency and a long lifetime, and a material suitable therefor. A mixed composition comprising a compound represented by the general formula (1) and a compound represented by the general formula (2), wherein the compound represented by the general formula (1) is an indolocarbazole compound having a structure in which a substituted triazine ring is bound to one N of an indolocarbazole ring and a carbazolyl group is bound to other N thereof, and the compound represented by the general formula (2) is a biscarbazole compound having a structure in which an aromatic hydrocarbon group is bound to N in a carbazole ring by substitution.

### TECHNICAL FIELD

The present invention relates to a mixed composition, and an organic electroluminescent element or device (referred to as an organic EL device.) in which the mixed composition is used. The present invention specifically relates to a mixed composition of an indolocarbazole compound and a biscarbazole compound, and an organic EL device in which the mixed composition is used.

### BACKGROUND ART

Application of a voltage to an organic electroluminescent element or device (referred to as an organic EL device.) allows injection of holes and electrons from an anode and a cathode, respectively, into a light-emitting layer. Then, in the light-emitting layer, injected holes and electrons recombine to generate excitons. At this time, according to statistical rules of electron spins, singlet excitons and triplet excitons are generated at a ratio of 1:3. Regarding a fluorescence-emitting organic EL device using light emission from singlet excitons, it is said that the internal quantum efficiency thereof has a limit of 25%. Meanwhile, regarding a phosphorescent organic EL device using light emission from triplet excitons, it is known that intersystem crossing is efficiently performed from singlet excitons, the internal quantum efficiency is enhanced to 100%.

However, a technical object of a phosphorescent organic EL device is to increase the lifetime.

Moreover, highly efficient organic EL devices utilizing delayed fluorescence have been developed recently. For example, Patent Literature 1 discloses an organic EL device utilizing a TTF (Triplet-Triplet Fusion) mechanism, which is one of delayed fluorescence mechanisms. The TTF mechanism utilizes a phenomenon in which singlet excitons are generated due to collision of two triplet excitons, and it is thought that the internal quantum efficiency can be theoretically raised to 40%. However, since the efficiency is lower compared to phosphorescent organic EL devices, further improvement in efficiency is required.

Meanwhile, patent Literature 2 discloses an organic EL device utilizing a TADF (Thermally Activated Delayed Fluorescence) mechanism. The TADF mechanism utilizes a phenomenon in which reverse intersystem crossing from triplet excitons to singlet excitons is generated in a material having a small energy difference between a singlet level and a triplet level, and it is thought that the internal quantum efficiency can be theoretically raised to 100%. However, there is a demand for further improvement in lifetime characteristics, as in a phosphorescent device.

### CITATION LIST

### Patent Literature

Patent Literature 1: WO2010/134350 A
Patent Literature 2: WO2011/070963 A
Patent Literature 3: WO2013/088973 A
Patent Literature 4: JP2003-133075 A
Patent Literature 5: US2015/0236262 A
Patent Literature 6: WO2011/059463 A
Patent Literature 7: WO2016/194604 A
Patent Literature 8: WO2018/198844 A
Patent Literature 9: WO2018/061446 A
Patent Literature 10: WO2022/255243 A
Patent Literature 11: WO2023/287026 A
Patent Literature 12: WO2020/195917 A

Patent Literatures 3 discloses use of an indolocarbazole compound as a host material. Patent Literature 4 discloses use of a biscarbazole compound as a host material.

Patent Literature 5 discloses use of an indolocarbazole compound and a biscarbazole compound as a mixed host. Patent Literatures 6 discloses use of a deuterated indolocarbazole compound as a host material.

Patent Literatures 7, 8, and 9 disclose use of a mixed composition of plural indolocarbazole compounds and biscarbazole compounds, as a host material. Patent Literature 12 discloses use of a molten mixture obtained by heating and melting an indolocarbazole compound and a biscarbazole compound in advance before vapor deposition, as a host.

Patent Literatures 10 and 11 disclose use of a mixed composition of plural indolocarbazole compounds and deuterated biscarbazole compounds, as a host material.

However, none of these can be said to be sufficient in that a device achieves a reduction in driving voltage, an enhancement in luminous efficiency, and an increase in lifetime, and further improvement is desired.

### SUMMARY OF INVENTION

### Technical Problem

In order to apply organic EL devices to display devices such as flat panel displays, it is necessary to improve the luminous efficiency of devices and at the same time, to sufficiently secure the lifetime characteristics of the devices. In view of the above circumstances, an object of the present invention is to provide a practically useful organic EL device having a low driving voltage and also having a high efficiency and a long lifetime, and a compound suitable therefor.

### Solution to Problem

As a result of intensive studies, the present inventors have found that excellent characteristics are exhibited by use of a mixed composition of specific indolocarbazole compound and biscarbazole compound, for an organic EL device, and have completed the present invention.

The present invention relates to a mixed composition comprising a compound represented by the general formula (1) and a compound represented by the following general formula (2):

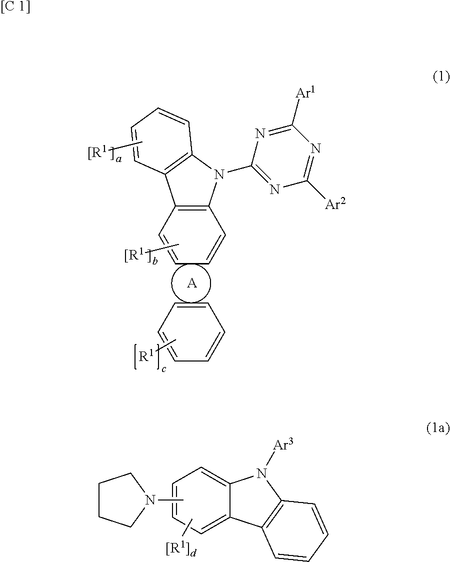

wherein a ring A is a 5-membered heterocycle fused to two adjacent rings at any positions and represented by formula (1a); Ar¹ and Ar² each independently represent a substituted or unsubstituted phenyl group, a substituted or unsubstituted biphenyl group, or a substituted or unsubstituted terphenyl group; Ar³ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other, and aromatic hydrocarbon groups or aromatic heterocyclic groups in the case of these aromatic groups linked are the same as or different from each other;

Each R¹ independently represents a substituted or unsubstituted aromatic hydrocarbon group having 6 to 10 carbon atoms, a substituted or unsubstituted aromatic heterocyclic group having 3 to 12 carbon atoms or a substituted or unsubstituted linked aromatic group in which two to five of these aromatic groups are linked to each other, and aromatic hydrocarbon groups or aromatic heterocyclic groups in the case of these aromatic groups linked are the same as or different from each other.

a to d represent the number of substitutions, a and c each represent an integer of 0 to 4, b represents an integer of 0 to 2, and d represents an integer of 0 to 3, preferably a and c each represent an integer of 0 to 2, b represents an integer of 0 to 1, and d represents an integer of 0 to 1;

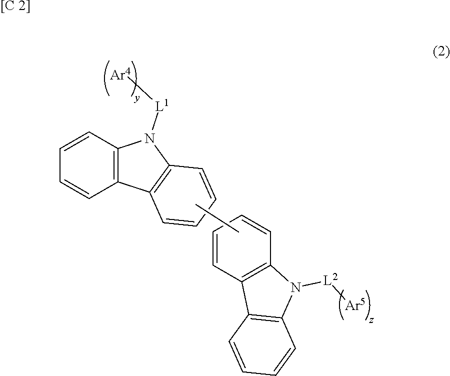

wherein Ar⁴ and Ar⁵ each independently represent a substituted or unsubstituted aromatic hydrocarbon group having 6 to 14 carbon atoms, or a substituted or unsubstituted linked aromatic group in which two of these aromatic hydrocarbon groups are linked to each other, and aromatic hydrocarbon groups in the case of these aromatic rings linked are the same as or different from each other; Ar⁴ and Ar⁵ preferably represent a substituted or unsubstituted phenyl group, a substituted or unsubstituted biphenyl group, a substituted or unsubstituted naphthyl group, or a substituted or unsubstituted phenanthryl group, more preferably represents an unsubstituted phenyl group.

L¹ and L² each independently represent a direct bond, or a substituted or unsubstituted phenylene group. y and z each independently represent an integer of 1 to 2, preferably satisfy y=z=1. When y or z represents 2, L¹ or L² does not represent a direct bond and the phenylene group is a trivalent phenylene group.

The mixed composition preferably comprises 20 wt % or more and 70 wt % or less of the compound represented by the general formula (1) based on the compound represented by the general formula (1) and the compound represented by the general formula (2) in total.

The present invention relates to a mixed composition comprising the compound represented by the general formula (1) as a first host and the compound represented by the general formula (2) as a second host. The mixed composition includes a premixture used as a mixed host and a mixture present in a vapor-deposited film produced with the mixed host. The vapor-deposited film comprises a light-emitting layer having a dopant (light-emitting dopant material).

Hydrogen in at least one compound of the compound represented by the general formula (1) or the compound represented by the general formula (2) is preferably partially or fully replaced by deuterium.

Hydrogen in at least one compound of the compound represented by the general formula (1) or the compound represented by the general formula (2) is preferably partially or fully replaced by deuterium, and the average rate of deuteration is preferably 30% or more, more preferably 403 or more.

The mixed composition can be a premixture which is a material for producing at least one layer in an organic electroluminescent device by a vapor deposition method and which is formed by previous mixing before vapor deposition, or which is formed by previous mixing by heating and melting before vapor deposition.

In the premixture, the difference in 50% weight reduction temperatures of the compound represented by the general formula (1) and the compound represented by the general formula (2) may be within 20° C.

The present invention relates to an organic EL device comprising a plurality of organic layers between an anode and a cathode, wherein at least one of the organic layers contains the mixed composition.

The organic layer containing the mixed composition is at least one selected from the group consisting of a light-emitting layer, a hole injection layer, a hole transport layer, an electron transport layer, an electron injection layer, a hole blocking layer and an electron blocking layer, more preferably a light-emitting layer. When the organic layer containing the mixed composition is a light-emitting layer, the light-emitting layer preferably contains at least one light-emitting dopant, and further preferably the compound represented by the general formula (1) as a first host and the compound represented by the general formula (2) as a second host are included and the light-emitting layer contains at least one light-emitting dopant.

The present invention also relates to a method for producing an organic electroluminescent device comprising a plurality of organic layers including a light-emitting layer between an anode and a cathode, the method comprising the steps of: providing the mixed composition, and vapor-depositing this mixed composition from one vapor deposition source to form a light-emitting layer.

### Advantageous Effects of Invention

In order to improve characteristics of an organic EL device, it is necessary to allow the durability of a material used for an organic layer, against charges, to be high and in particular it is important to suppress leakage of excitons and charges in a light-emitting layer to a peripheral layer. It is effective for such suppression of leakage of charges/excitons to improve the disproportionation of a light-emitting region in the light-emitting layer, and it is necessary for such an improvement to control the amounts of both charges (electron/hole) injected into the light-emitting layer or transport properties of both charges in the light-emitting layer to preferred ranges.

Injection/transport properties of both charges in the material used for an organic layer largely depend on the energy level and the intermolecular interaction of the molecular orbital of the material. The mixed composition of the present invention, when used in a material for an organic EL device, is high particularly in electron injection/transport ability because of including an indolocarbazole compound having a carbazolyl group represented by formula (1a), whereas can inhibit indolocarbazole molecules from being close to each other by the effect of steric hindrance of the carbazolyl group.

It is then considered that the changes in type of a substituent on N of the carbazolyl group and in binding position of the carbazolyl group with indocarbazole can allow for control of the intermolecular interaction of the molecular orbital largely contributing to electron injection/transport to a light-emitting layer, at a high level, and provides an excellent organic EL device.

Furthermore, an indolocarbazole compound and a biscarbazole compound can be mixed and used to more precisely regulate the amount of charge injection into an organic layer as compared with the case of single use of each of the compounds. In particular, in the case of use in a light-emitting layer, a delayed fluorescence-emitting EL device or a phosphorescent EL device has a sufficiently high lowest excited triplet energy for confining the excitation energy generated in the light-emitting layer and thus causes no energy drain from the light-emitting layer and can achieve not only a low voltage, but also a high efficiency and a long lifetime.

### BRIEF DESCRIPTION OF DRAWING

FIG. 1 is a cross-sectional view showing one structure example of an organic EL device.

### DESCRIPTION OF EMBODIMENTS

The mixed composition of the present invention comprises the compound represented by the general formula (1) and the compound represented by the general formula (2).

In the general formula (1), a ring A is a 5-membered heterocycle represented by formula (1a), and the heterocycle is fused to two adjacent rings at any positions, but is not fused at a side containing N. Hence, an indolocarbazole ring has some isomer structures, but the number of such structures is restricted.

Specifically, there is an aspect in which the compound represented by the general formula (1) has a structure represented by any of the following formulas (3) to (8), preferably the formulas (6) to (8), more preferably the formula (8). In the formulas (3) to (8), the same symbol as that in the general formula (1) has the same meaning as that in the general formula (1).

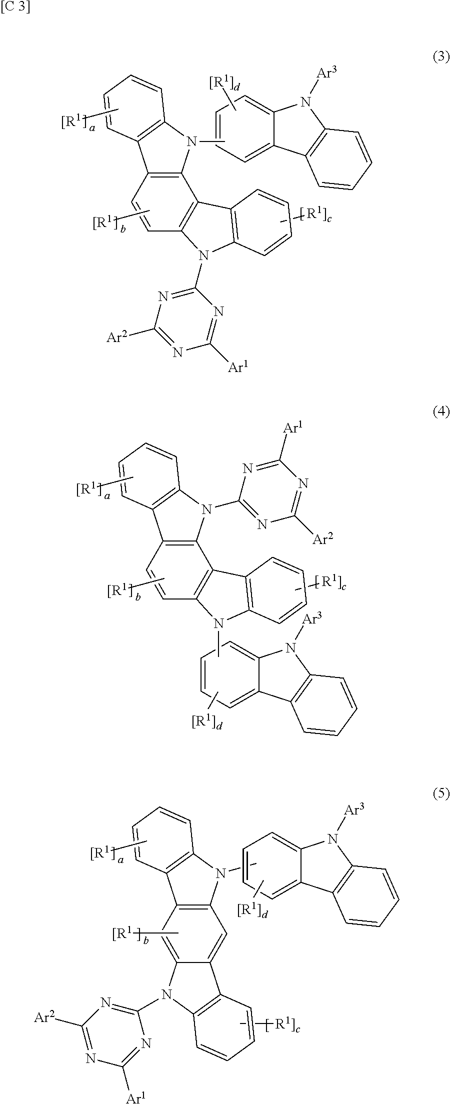

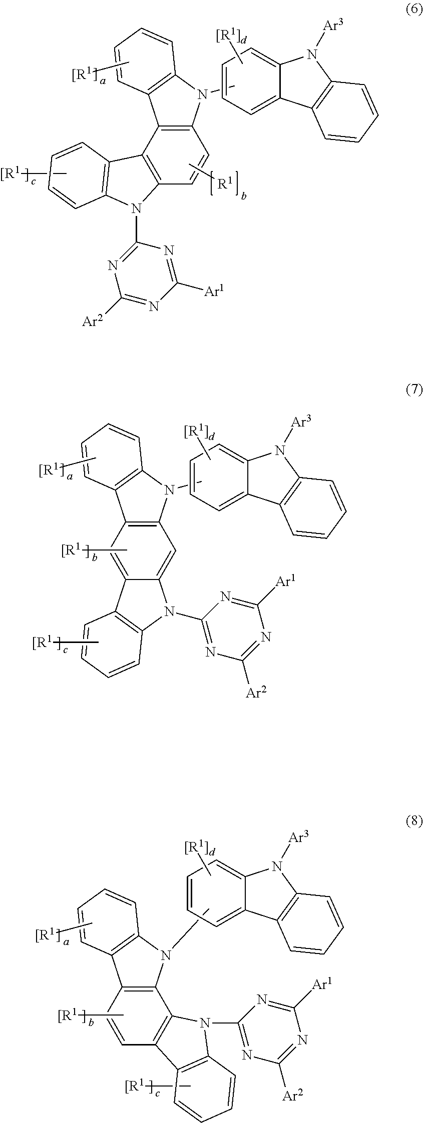

The substitution position of the carbazolyl group to indolocarbazole in the formula (1a) is preferably as represented by any of formulas (9) to (11), more preferably the formula (10) or (11). In the formulas (9) to (11), the same symbol as that in the formula (1a) has the same meaning as that in the formula (1a).

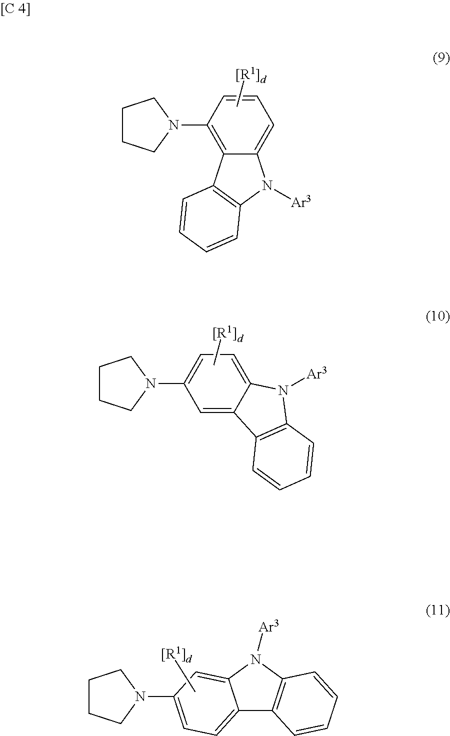

In the general formula (1), Ar¹ and Ar² are each independently a substituted or unsubstituted phenyl group, a substituted or unsubstituted biphenyl group, or a substituted or unsubstituted terphenyl group; and the sum of the numbers of benzene rings in Ar¹ and Ar² is preferably 4 or less, more preferably 3 or less. For example, the biphenyl group has a structure in which two benzene rings are linked, the terphenyl group has a structure in which three benzene rings are linked, and the binding position of each of the benzene rings may be any of the o-, m-, and p-positions. These groups may be each linear or branched.

Ar³ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other, preferably represents a substituted or unsubstituted aromatic hydrocarbon group having 6 to 12 carbon atoms, a substituted or unsubstituted aromatic heterocyclic group having 5 to 11 carbon atoms, or a substituted or unsubstituted linked aromatic group in which two of these aromatic hydrocarbon groups are linked to each other.

Specific examples of the unsubstituted aromatic hydrocarbon group having 6 to 18 carbon atoms, the unsubstituted aromatic heterocyclic group having 3 to 17 carbon atoms, or the unsubstituted linked aromatic group in which two to five of these aromatic groups are linked to each other include a group generated by removing one hydrogen from benzene, pentalene, indene, naphthalene, anthracene, phenanthrene, pyrrole, imidazole, pyrazole, thiazole, thiophene, pyridine, pyrazine, pyrimidine, pyridazine, triazine, isoindole, indazole, purine, benzimidazole, indolizine, chromene, benzoxazole, isobenzofuran, quinolizine, isoquinoline, imidazole, naphthyridine, phthalazine, quinazoline, quinoxaline, cinnoline, quinoline, pteridine, perimidine, phenanthroline, phenanthridine, acridine, phenazine, phenothiazine, phenoxazine, dibenzodioxin, carboline, indole, carbazole, furan, benzofuran, isobenzofuran, benzothiazole, oxathrene, dibenzofuran, thiophene, thioxanthene, thianthrene, phenoxathiin, thionaphthene, isothianaphthene, thiophthene, thiophanthrene, dibenzothiophene, or compounds in which two to five of these are linked to each other.

Each R¹ independently represents a substituted or unsubstituted aromatic hydrocarbon group having 6 to 10 carbon atoms, a substituted or unsubstituted aromatic heterocyclic group having 3 to 12 carbon atoms, or a substituted or unsubstituted linked aromatic group in which two to five of these aromatic groups are linked to each other, and aromatic hydrocarbon groups or aromatic heterocyclic groups in the case of these aromatic groups linked are the same as or different from each other. Preferred is a substituted or unsubstituted phenyl group.

In the present specification, the linked aromatic group refers to an aromatic group in which the aromatic rings in two or more aromatic groups are linked to each other by a single bond. The linked aromatic group may be linear or branched. The linkage position in linking of benzene rings may be any of the ortho-, meta-, and para-positions, and is preferably the para-position or the meta-position. The aromatic group may be an aromatic hydrocarbon group or an aromatic heterocyclic group, and the plurality of aromatic groups may be the same or different.

Specific examples of R¹ which represents an unsubstituted aromatic hydrocarbon group having 6 to 10 carbon atoms, an unsubstituted aromatic heterocyclic group having 3 to 12 carbon atoms, or an unsubstituted linked aromatic group in which two to five of these aromatic groups are linked to each other include a group generated from benzene, naphthalene, pyridine, pyrimidine, triazine, thiophene, isothiazole, thiazole, pyridazine, pyrrole, pyrazole, imidazole, triazole, thiadiazole, pyrazine, furan, isoxazole, quinoline, isoquinoline, quinoxaline, quinazoline, thiadiazole, phthalazine, dibenzofuran, dibenzothiophene, dibenzoselenophene, carbazole, or compounds in which two to five of these are linked to each other.

In the present specification, the aromatic hydrocarbon group, aromatic heterocyclic group, or linked aromatic group may each have a substituent. In the case of having a substituent, the substituent is preferably deuterium, halogen, a cyano group, a triarylsilyl group, an aliphatic hydrocarbon group having 1 to 10 carbon atoms, an alkenyl group having 2 to 5 carbon atoms, an alkoxy group having 1 to 5 carbon atoms, or a diarylamino group having 12 to 44 carbon atoms. In the case of the substituent being an aliphatic hydrocarbon group having 1 to 10 carbon atoms, the substituent may be linear, branched, or cyclic. When the triarylsilyl group or the diarylamino group is the substituent of the aromatic hydrocarbon group, aromatic heterocyclic group, or linked aromatic group, silicon and carbon, or nitrogen and carbon are linked to each other by a single bond.

Note that the number of substituents is 0 to 5 and preferably 0 to 2. When the aromatic hydrocarbon group and the aromatic heterocyclic group have substituents, the calculation of the number of carbon atoms does not include the number of carbon atoms of the substituents. However, it is preferred that the total number of carbon atoms including the number of carbon atoms of the substituents satisfy the range.

Examples of such a substituent include deuterium, cyano, methyl, ethyl, propyl, i-propyl, butyl, t-butyl, pentyl, neopentyl, cyclopentyl, hexyl, cyclohexyl, heptyl, octyl, nonyl, decyl, vinyl, propenyl, butenyl, pentenyl, methoxy, ethoxy, propoxy, butoxy, pentoxy, diphenylamino, naphthylphenylamino, dinaphthylamino, dianthranylamino, diphenanthrenylamino, and dipyrenylamino. Examples preferably include deuterium, cyano, methyl, ethyl, t-butyl, propyl, butyl, pentyl, neopentyl, hexyl, heptyl, or octyldiphenylamino, naphthylphenylamino, or dinaphthylamino.

Specific examples of the general formula (1) are shown below, but are not limited to these exemplified compounds. The number of replacements by deuterium (D), for example, n, in the following structural formulas independently means the average number with respect to each compound, and is varied depending on the average rate of deuteration.

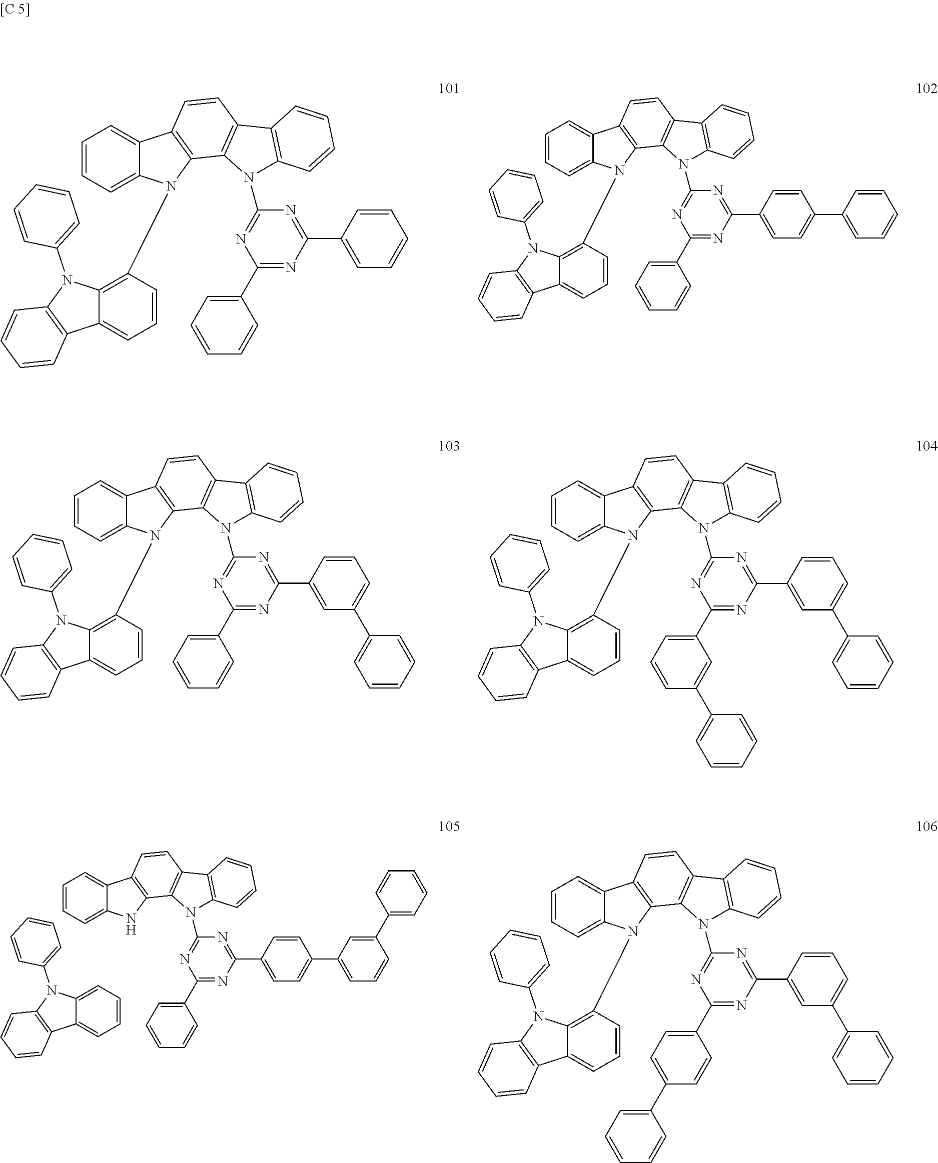

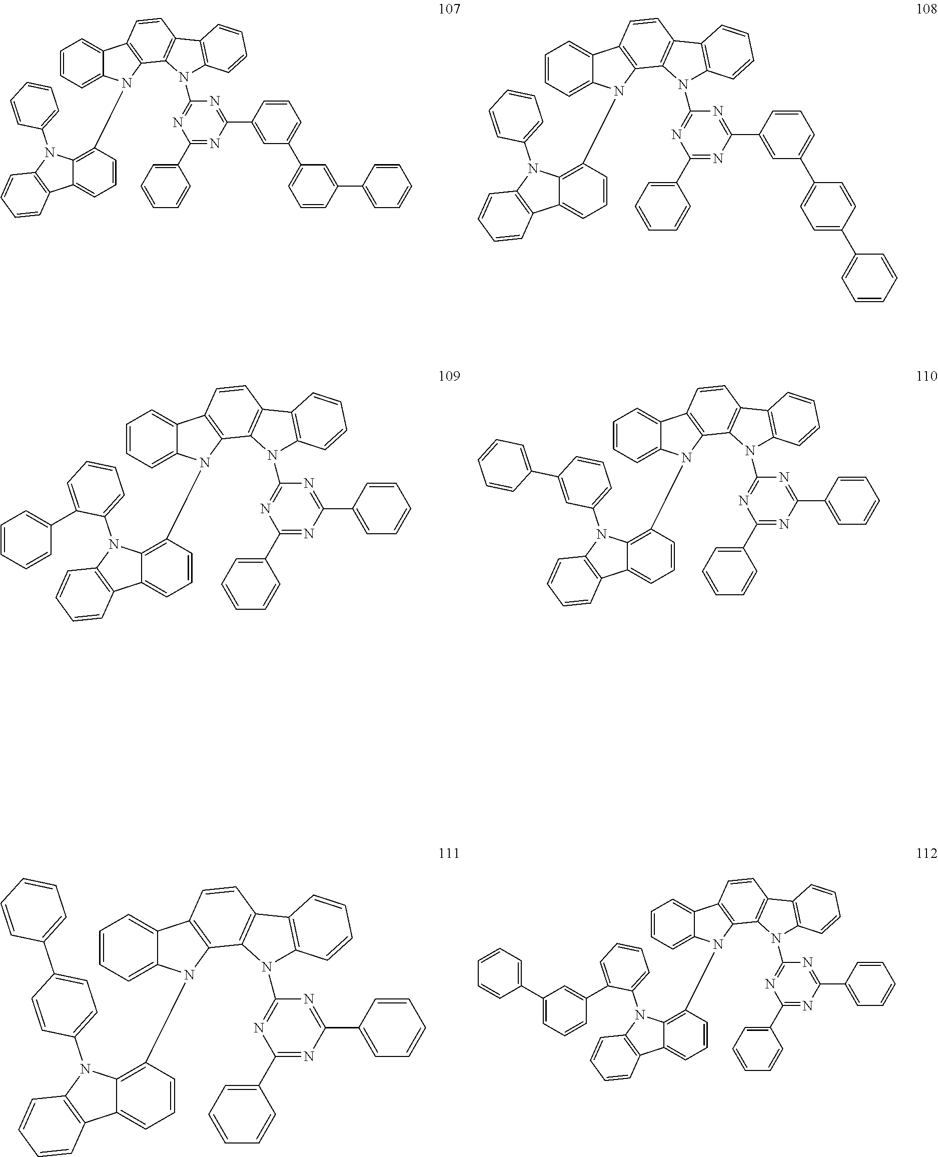

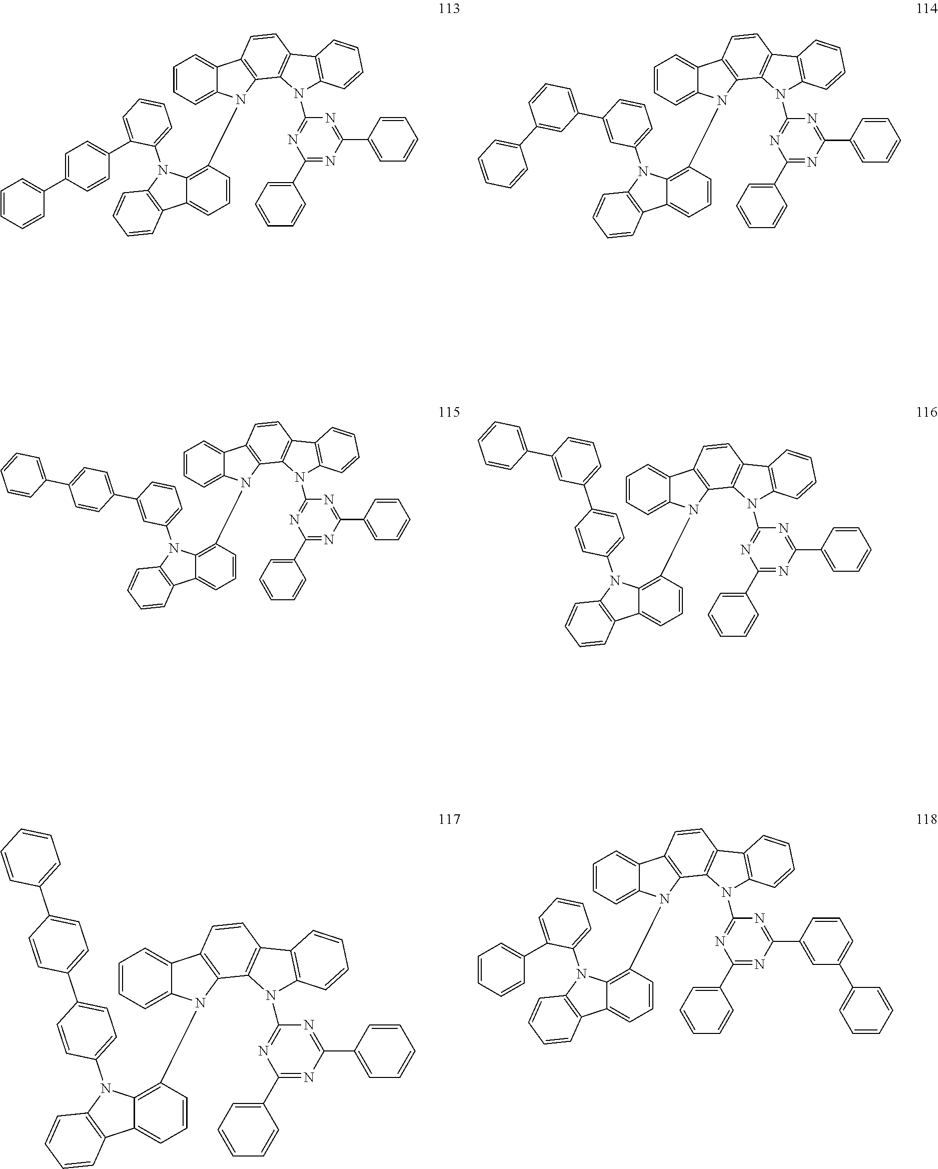

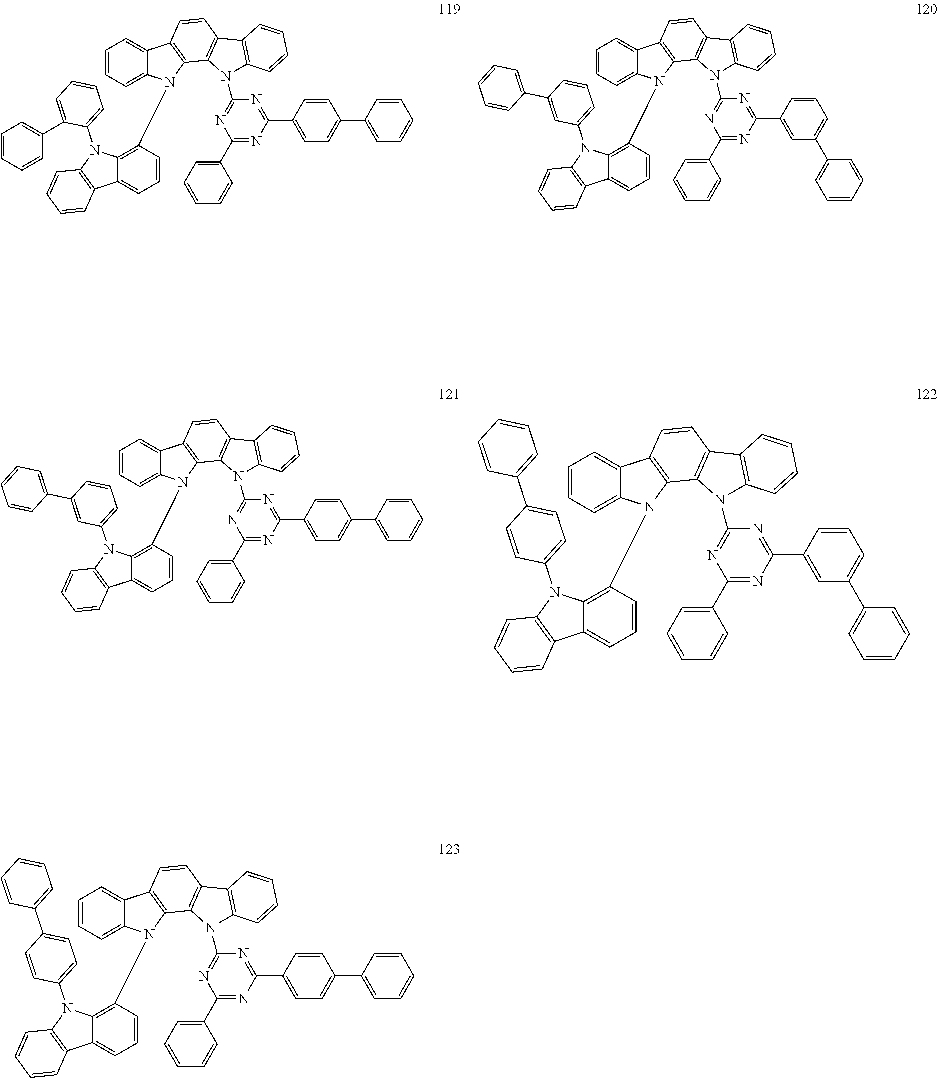

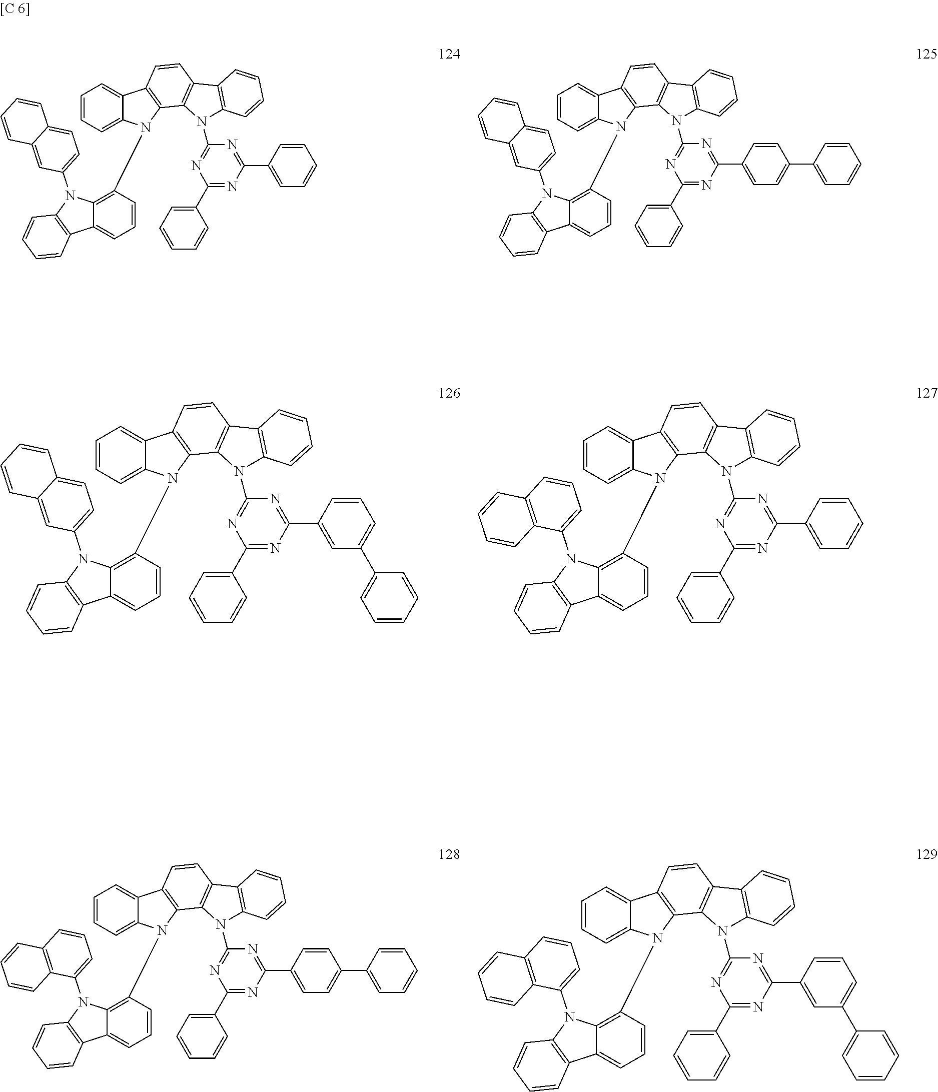

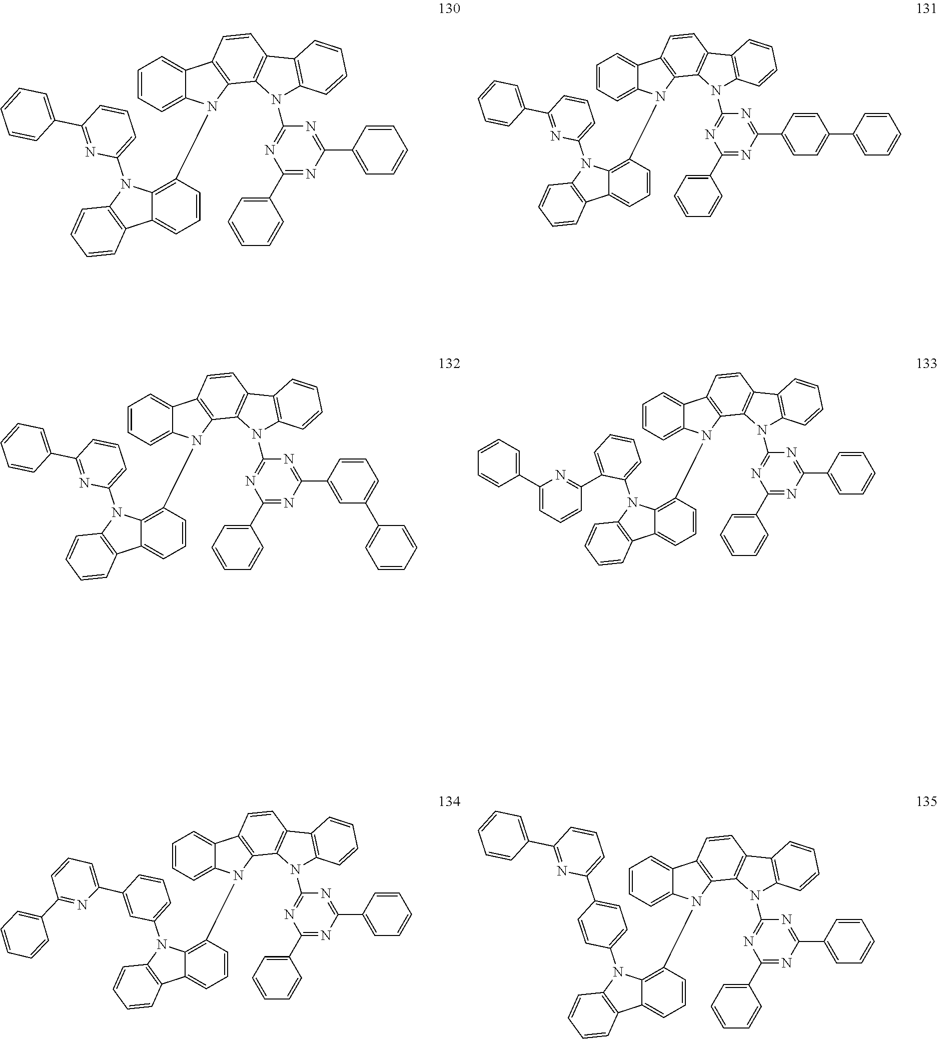

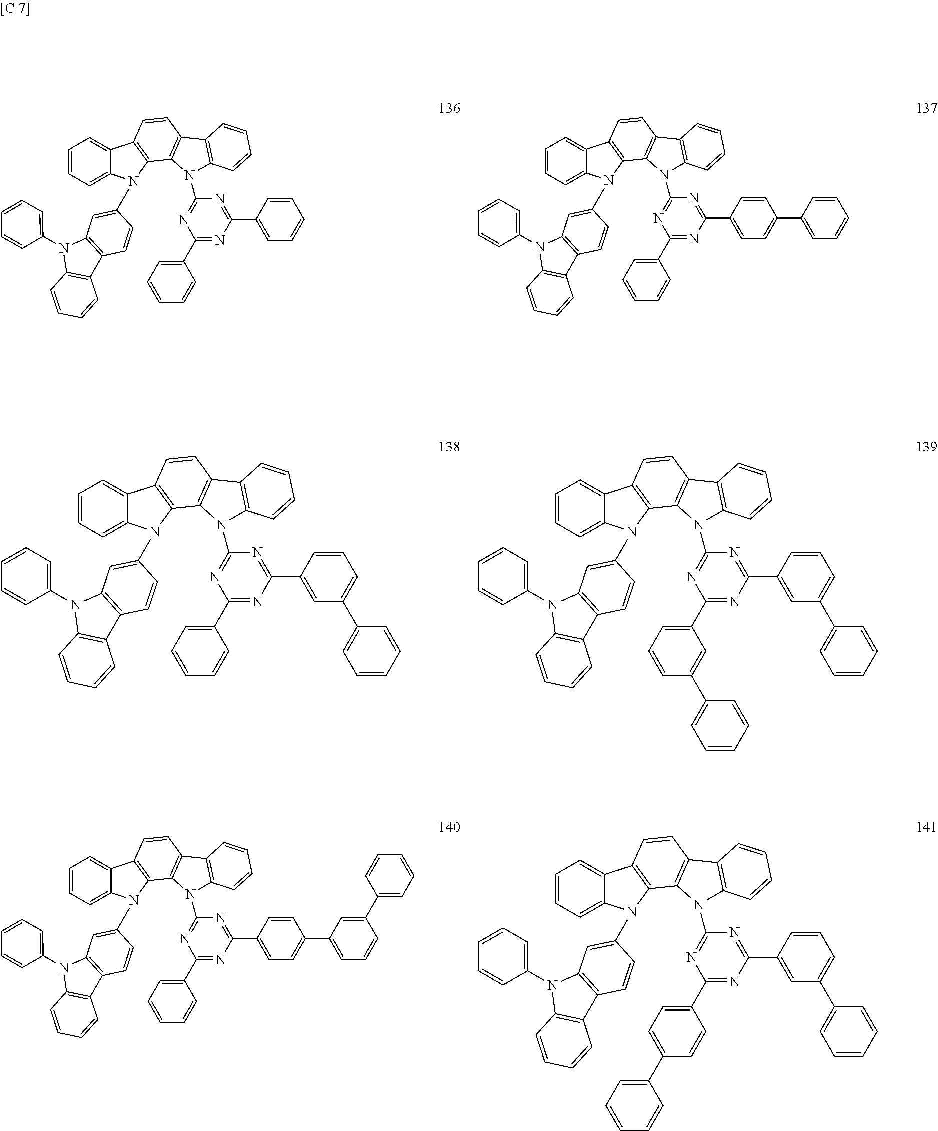

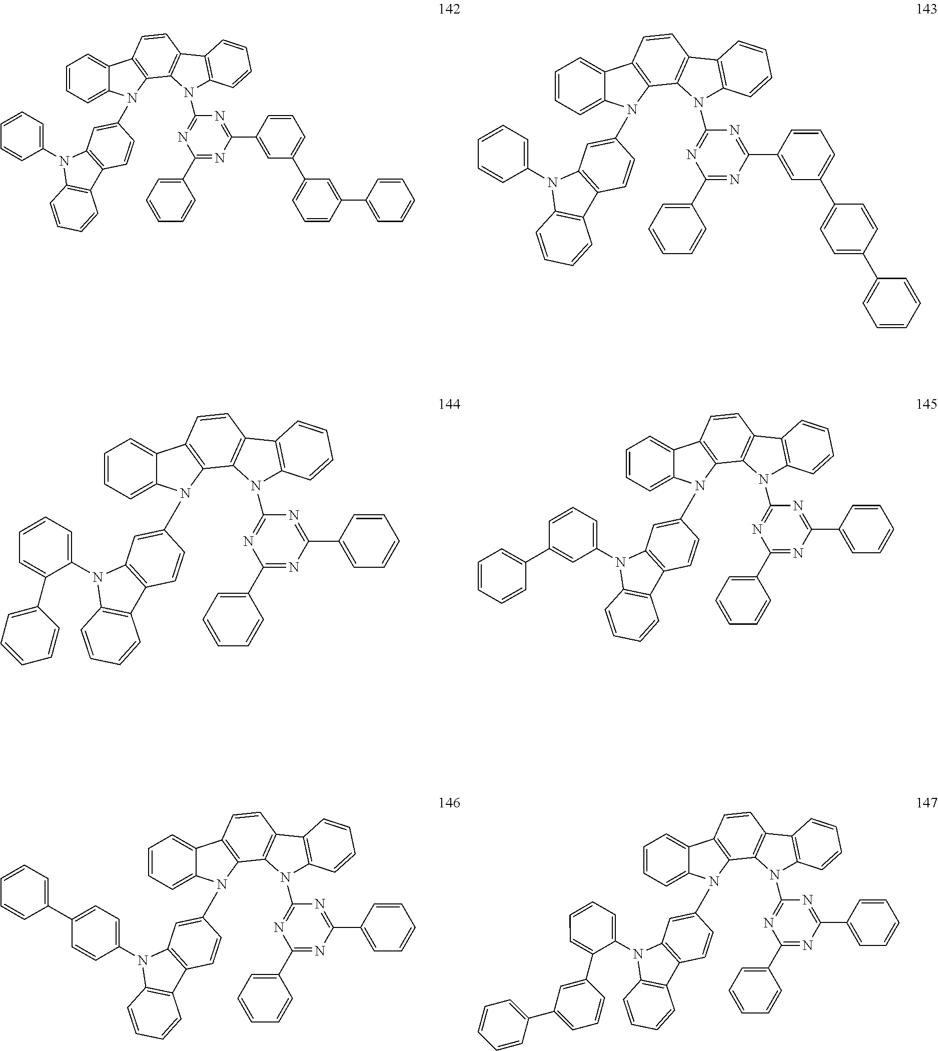

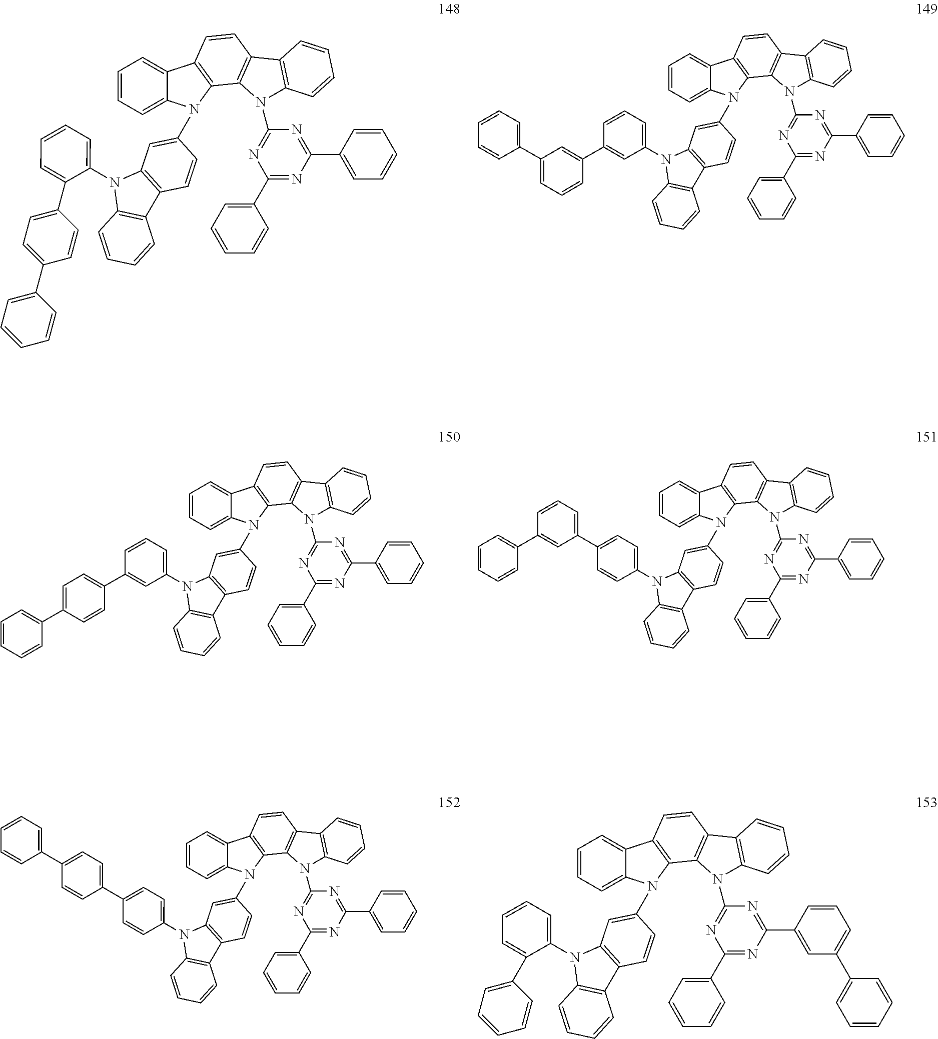

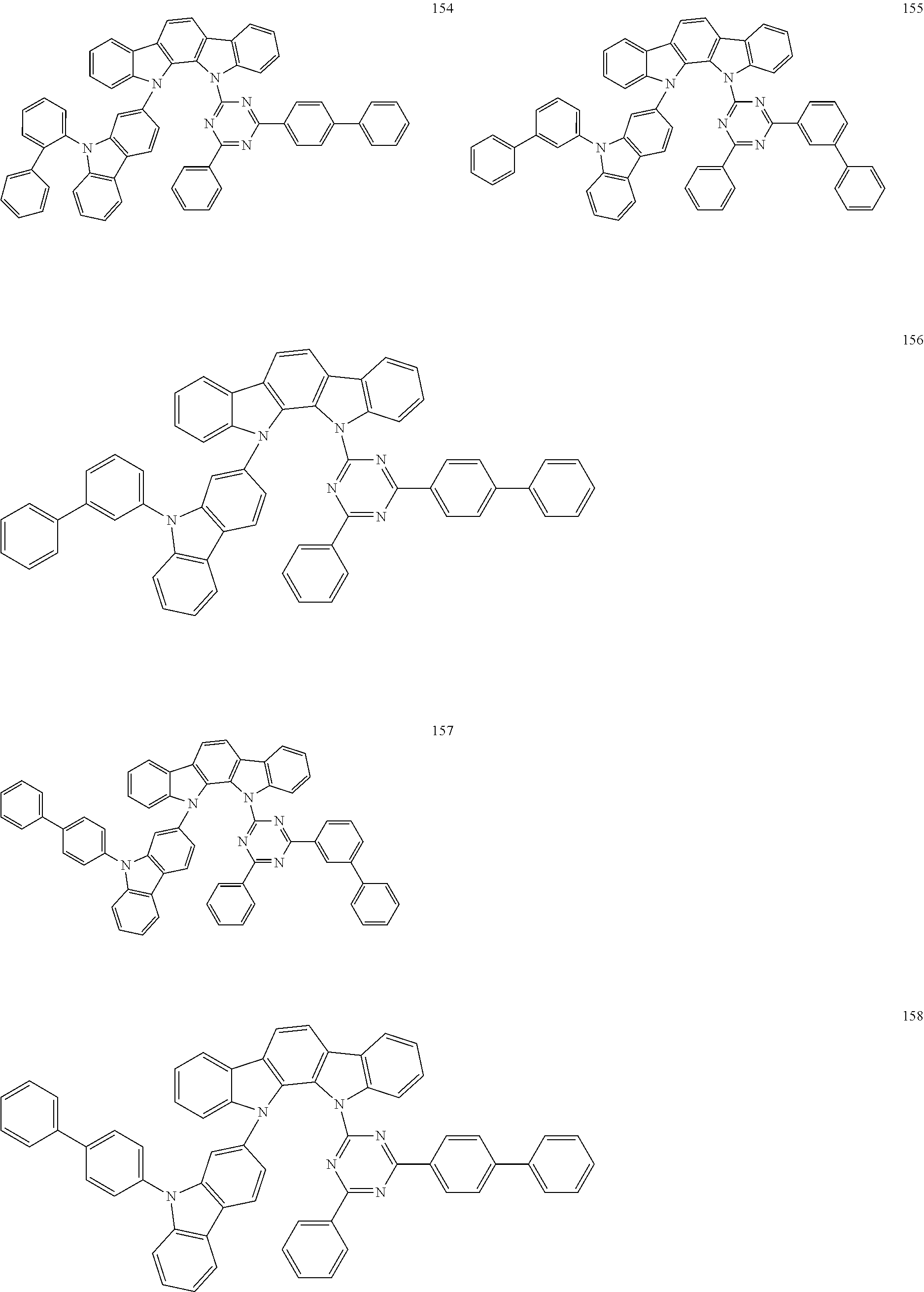

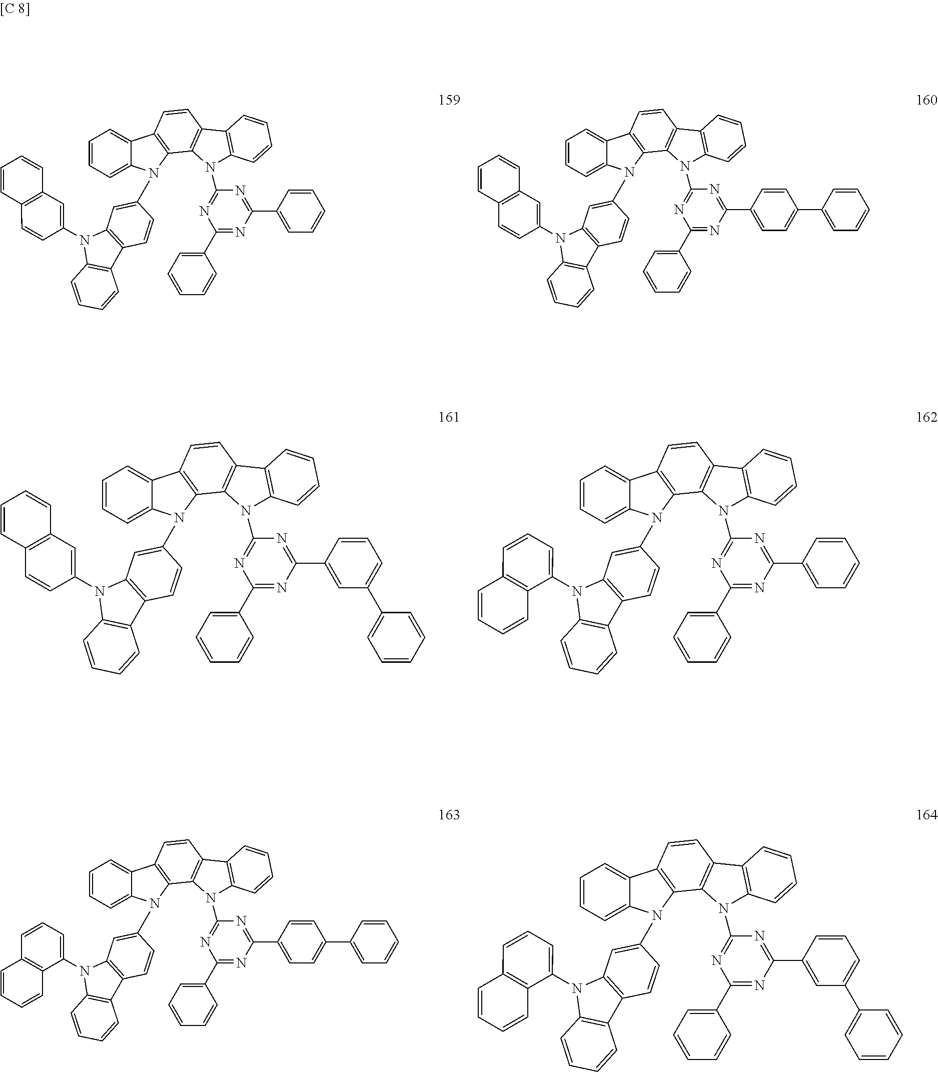

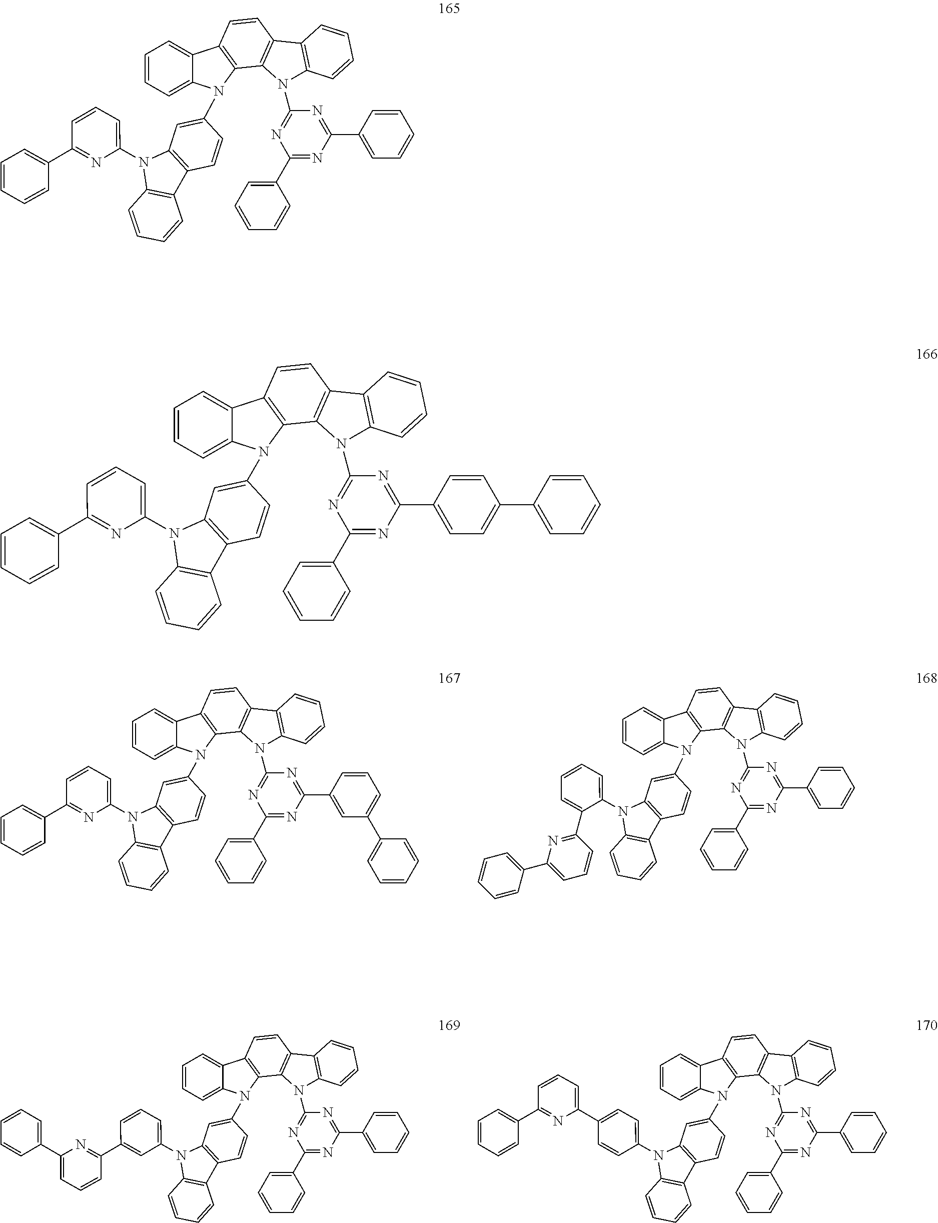

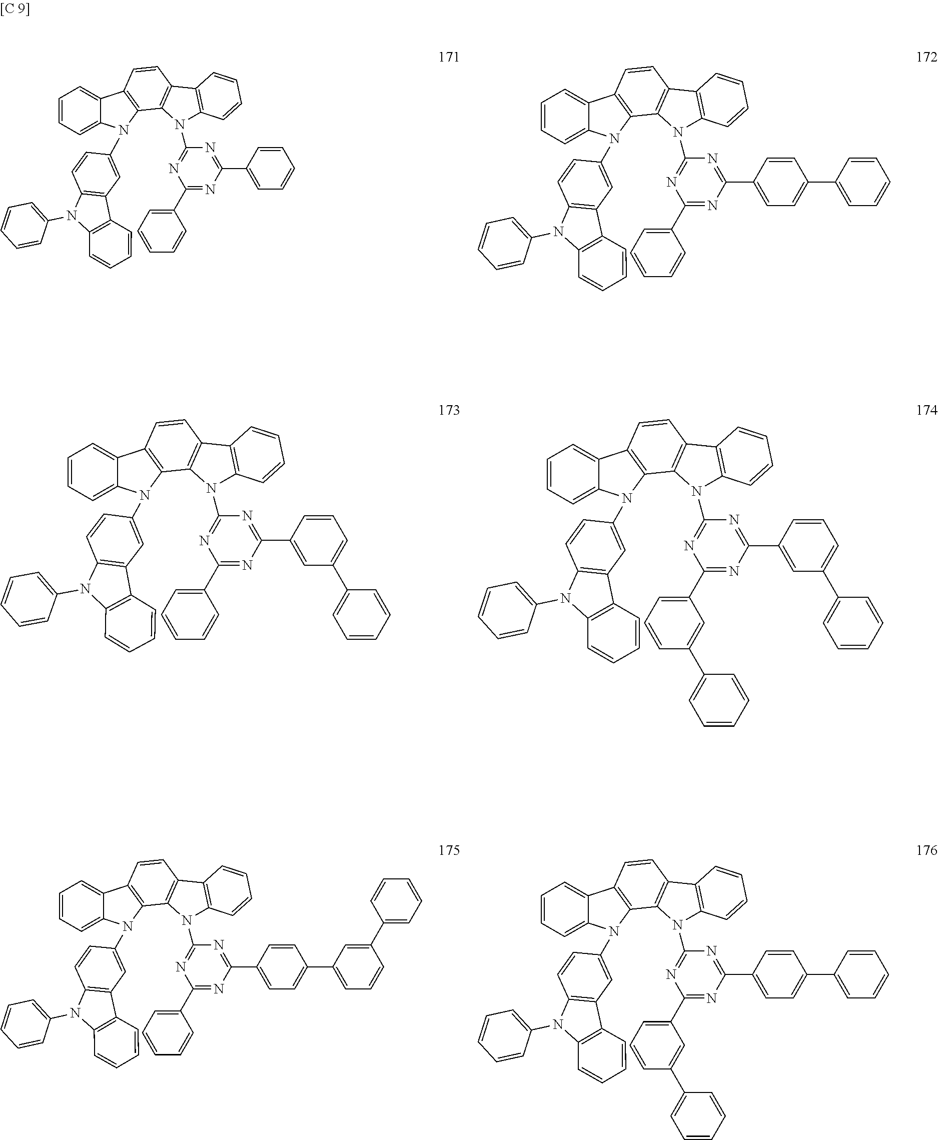

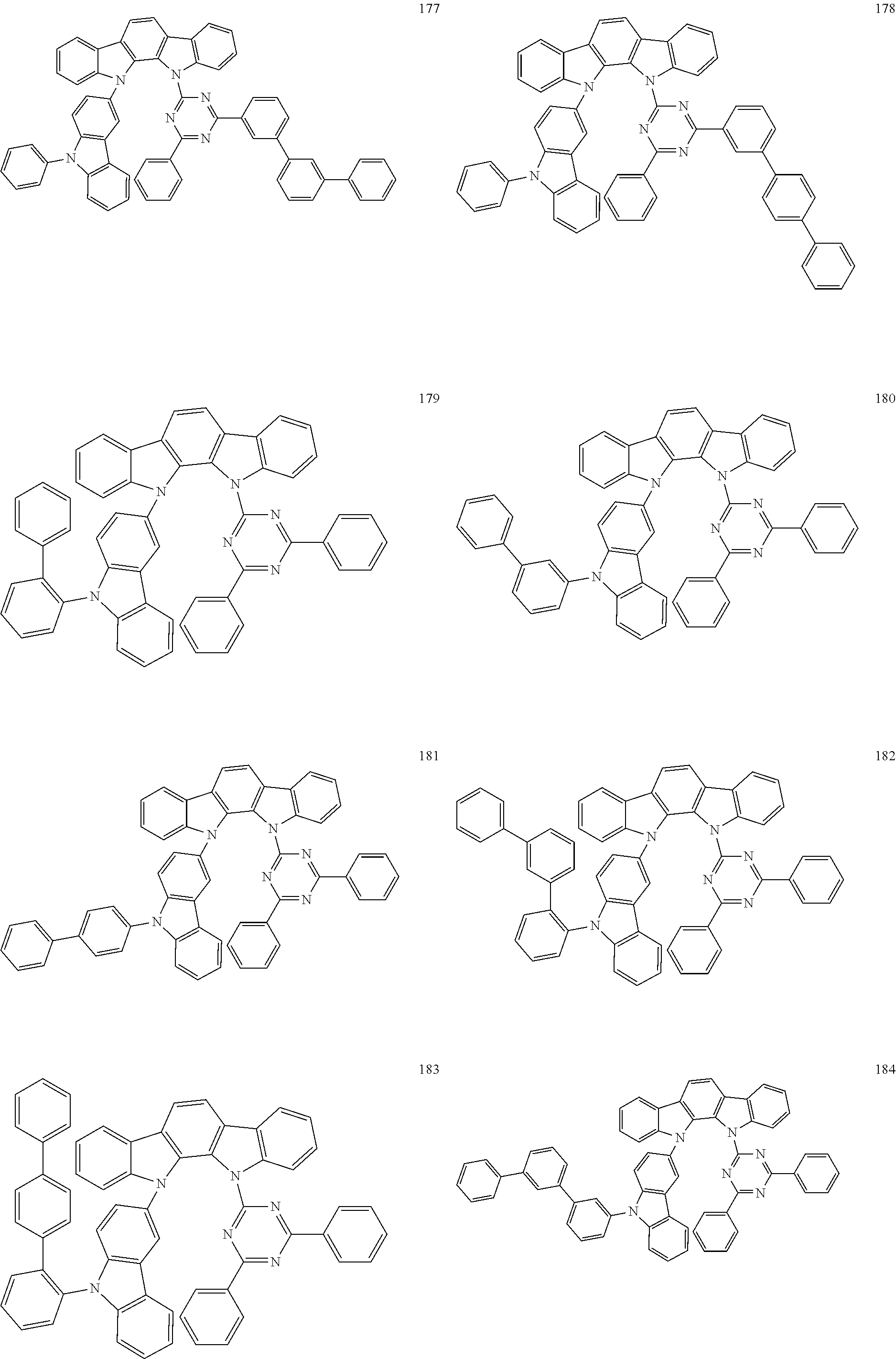

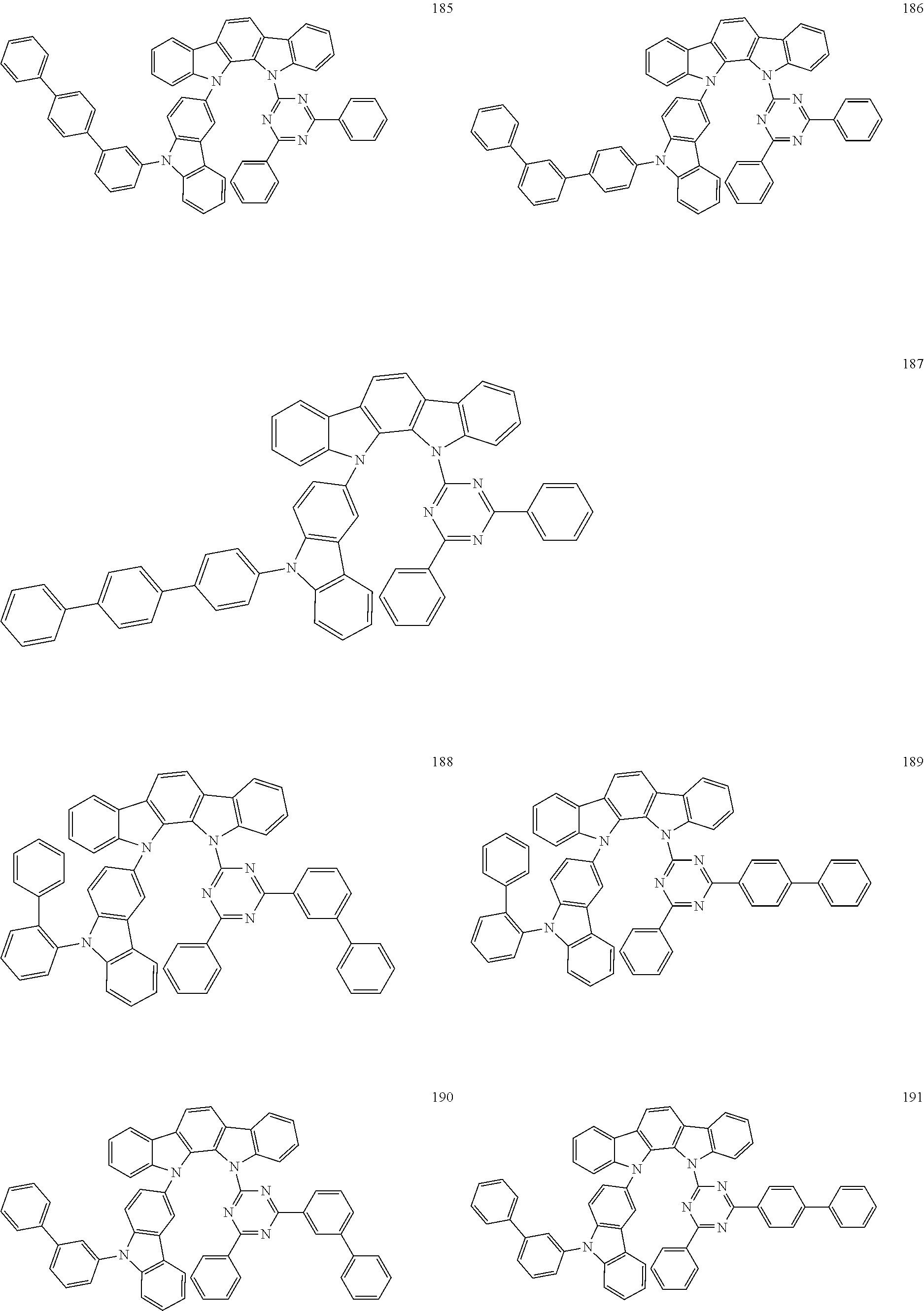

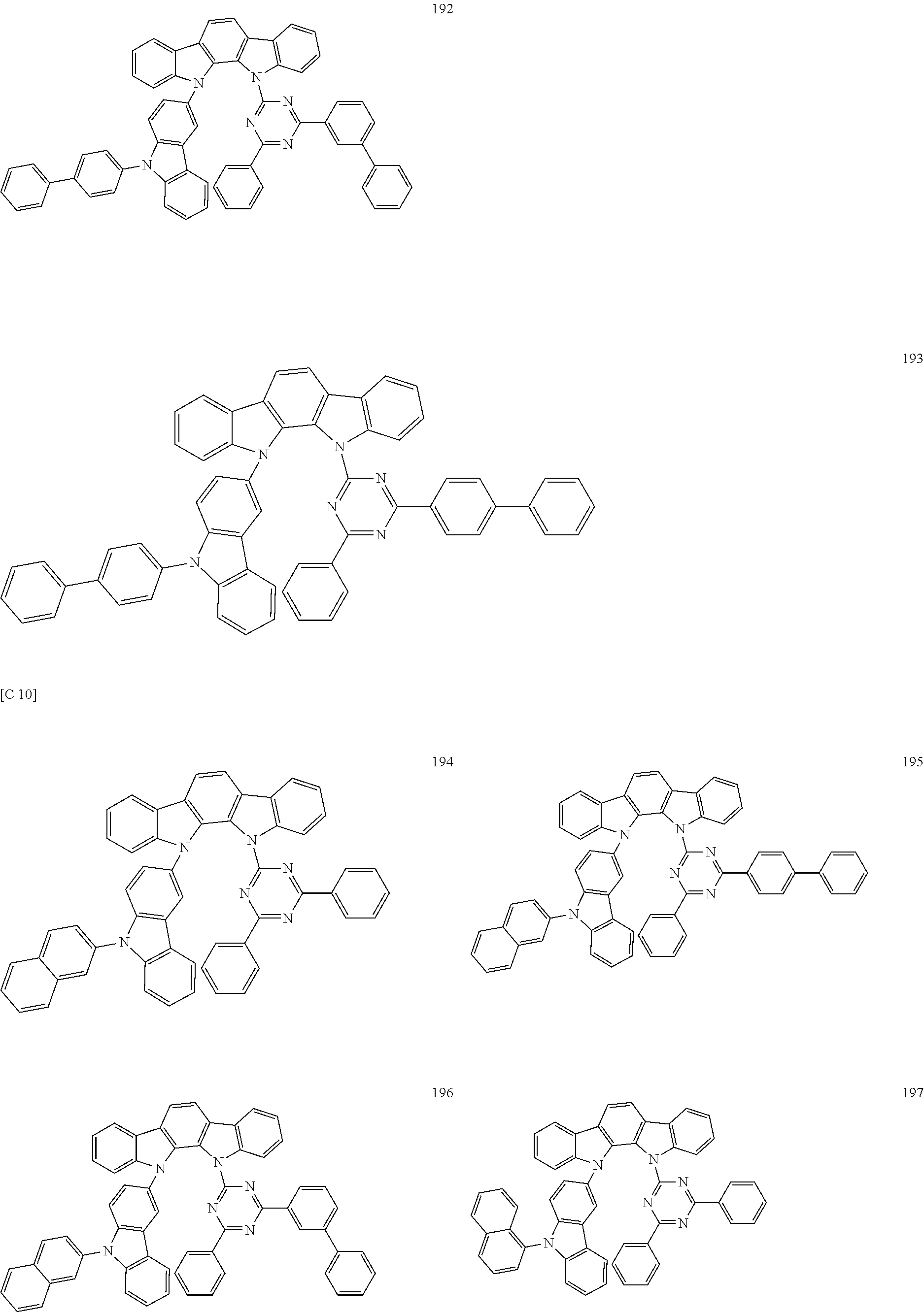

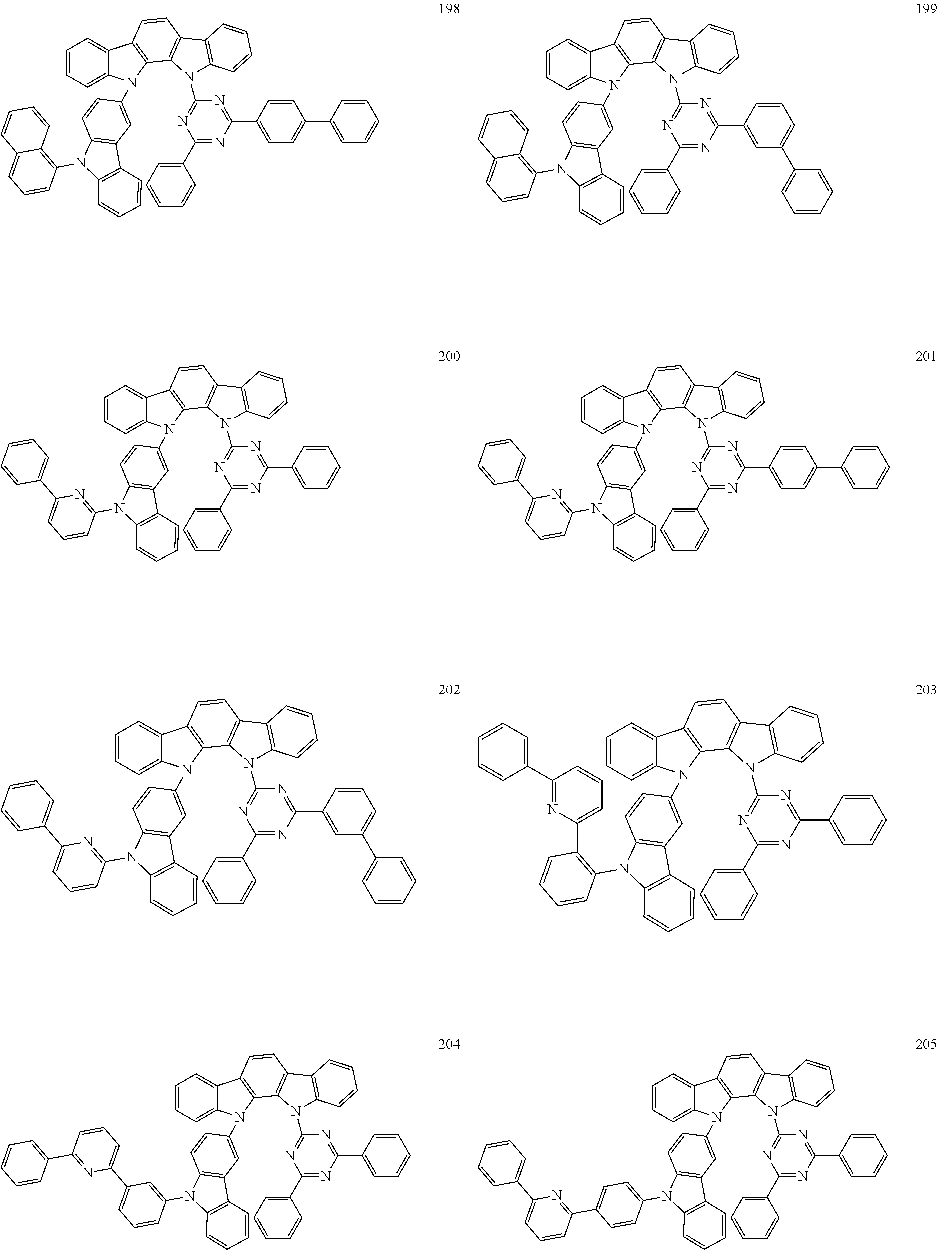

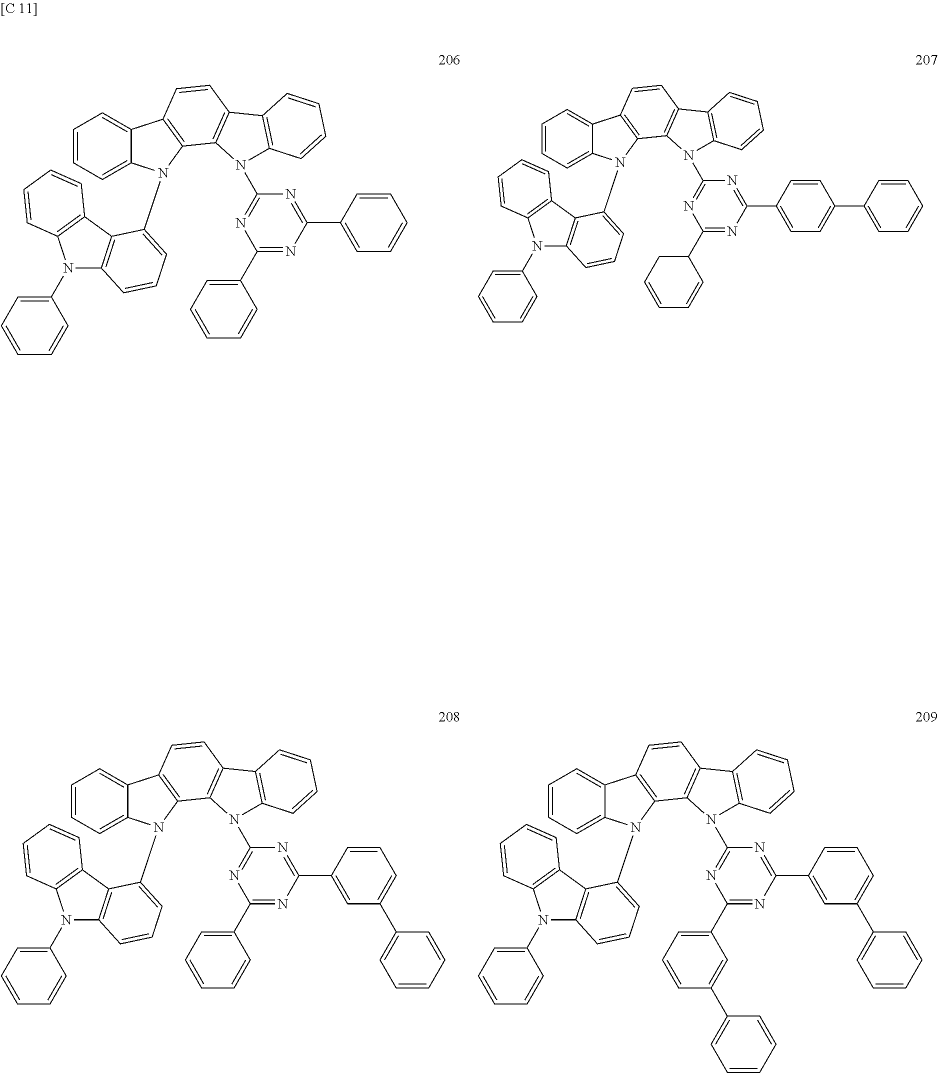

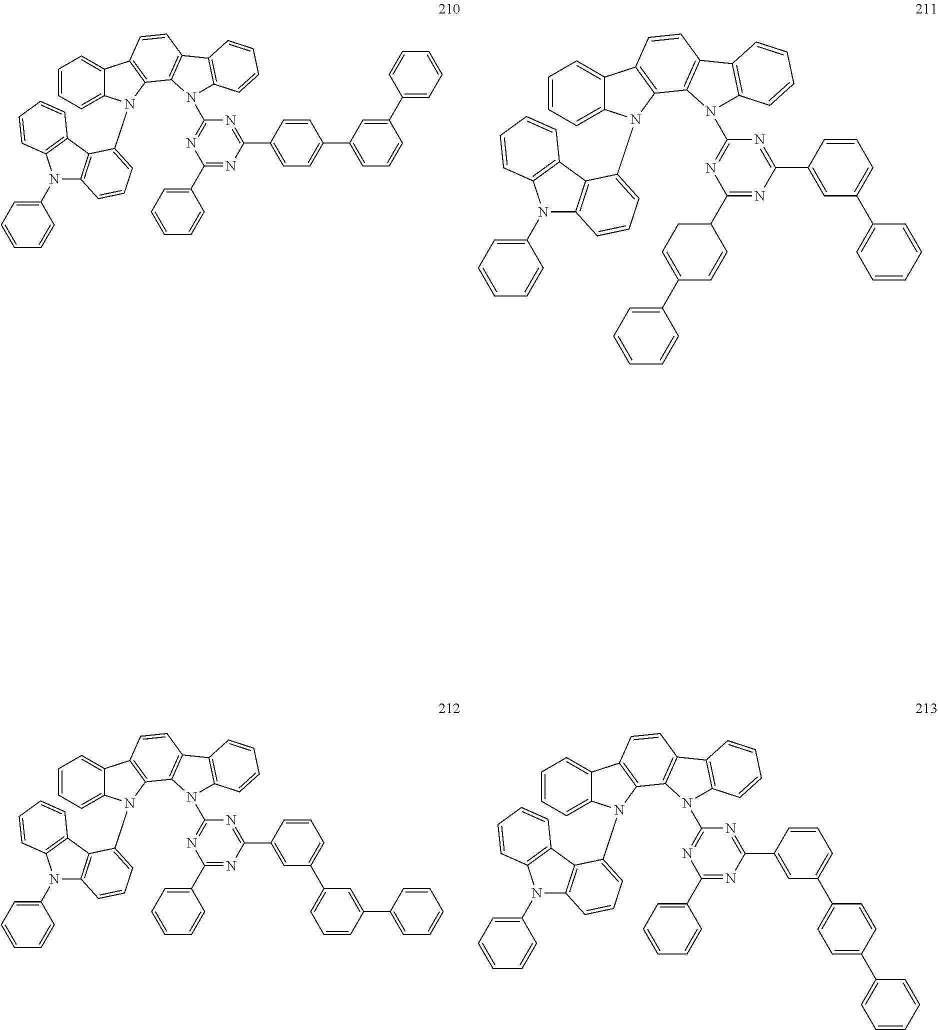 [SMILES: c1ccc(-c2cccc(-c3ccc(-c4nc(-c5ccccc5)nc(-n5c6ccccc6c6ccc7c8ccccc8n(-c8cccc9c8c8ccccc8n9-c8ccccc8)c7c65)n4)cc3)c2)cc1 ; C1=CC(c2nc(-c3cccc(-c4ccccc4)c3)nc(-n3c4ccccc4c4ccc5c6ccccc6n(-c6cccc7c6c6ccccc6n7-c6ccccc6)c5c43)n2)CC=C1c1ccccc1 ; c1ccc(-c2cccc(-c3cccc(-c4nc(-c5ccccc5)nc(-n5c6ccccc6c6ccc7c8ccccc8n(-c8cccc9c8c8ccccc8n9-c8ccccc8)c7c65)n4)c3)c2)cc1 ; c1ccc(-c2ccc(-c3cccc(-c4nc(-c5ccccc5)nc(-n5c6ccccc6c6ccc7c8ccccc8n(-c8cccc9c8c8ccccc8n9-c8ccccc8)c7c65)n4)c3)cc2)cc1]

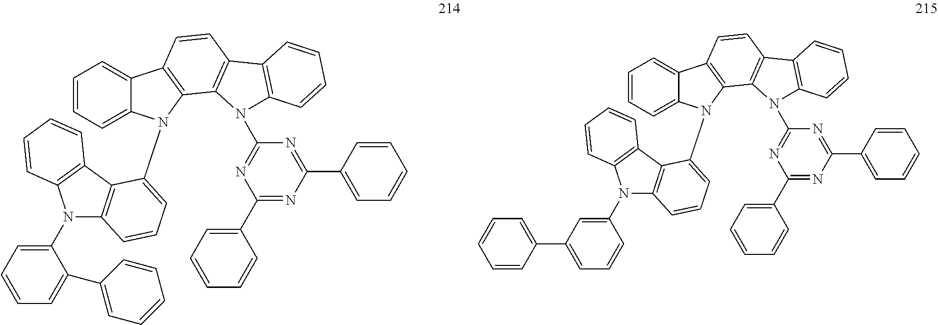 [SMILES: c1ccc(-c2nc(-c3ccccc3)nc(-n3c4ccccc4c4ccc5c6ccccc6n(-c6cccc7c6c6ccccc6n7-c6ccccc6-c6ccccc6)c5c43)n2)cc1 ; c1ccc(-c2cccc(-n3c4ccccc4c4c(-n5c6ccccc6c6ccc7c8ccccc8n(-c8nc(-c9ccccc9)nc(-c9ccccc9)n8)c7c65)cccc43)c2)cc1]

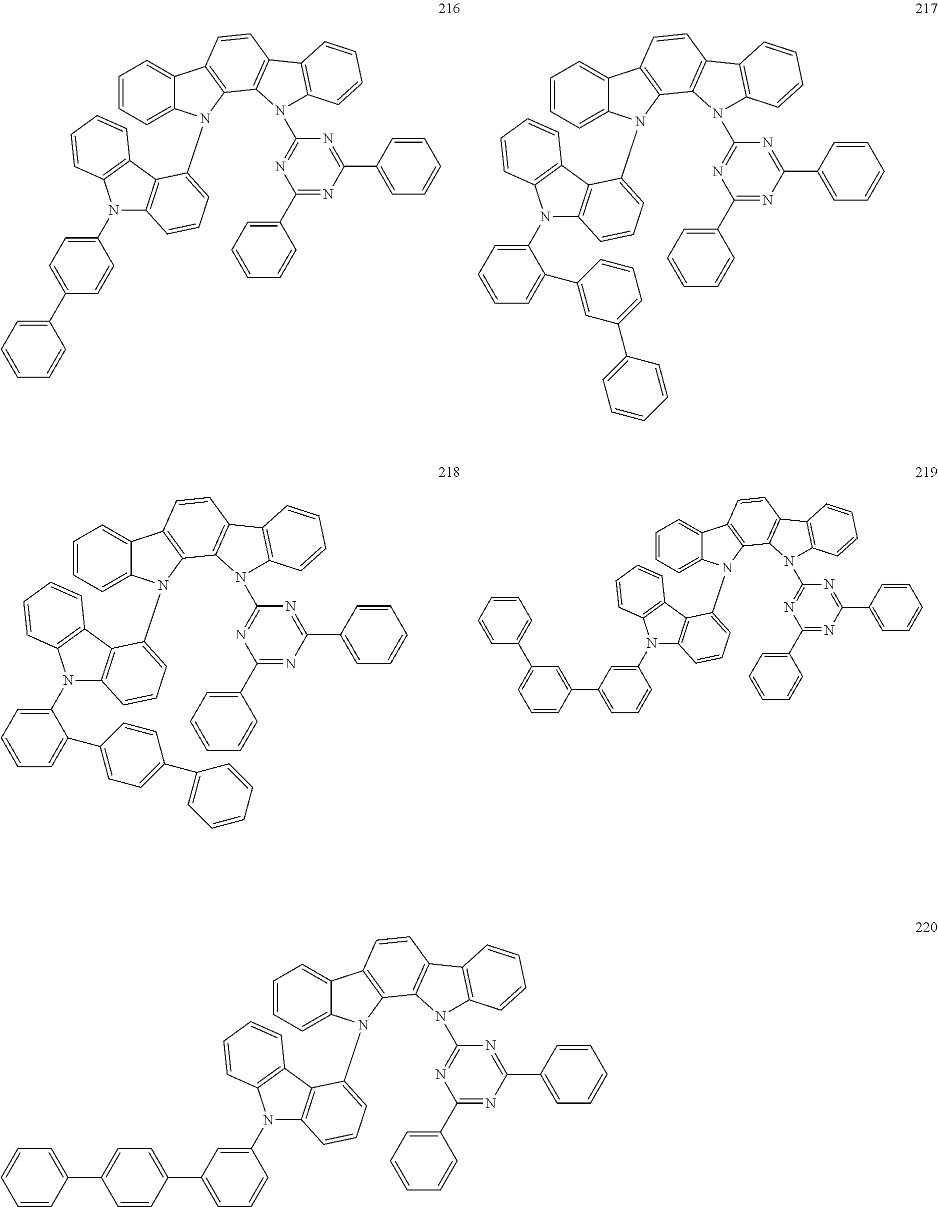

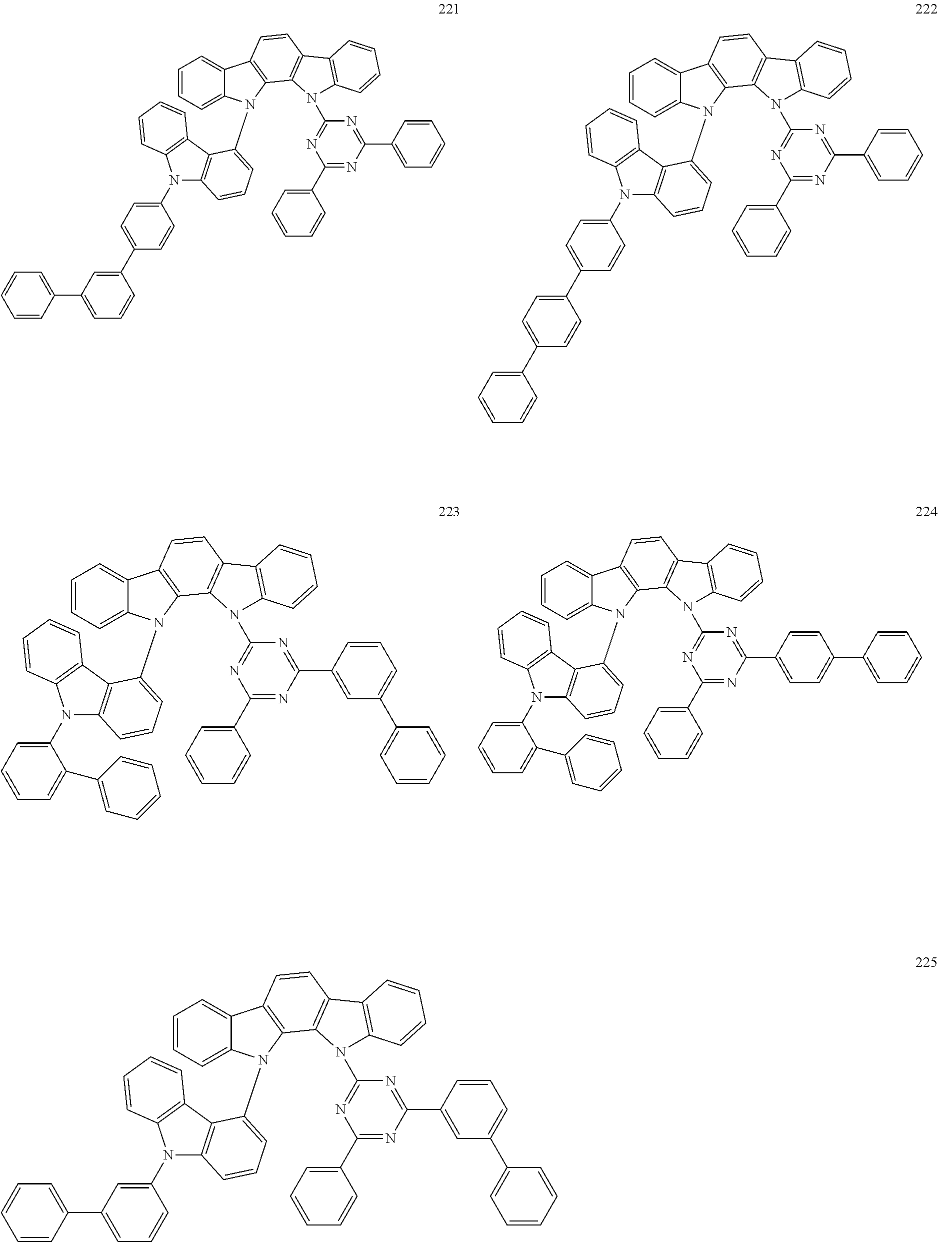

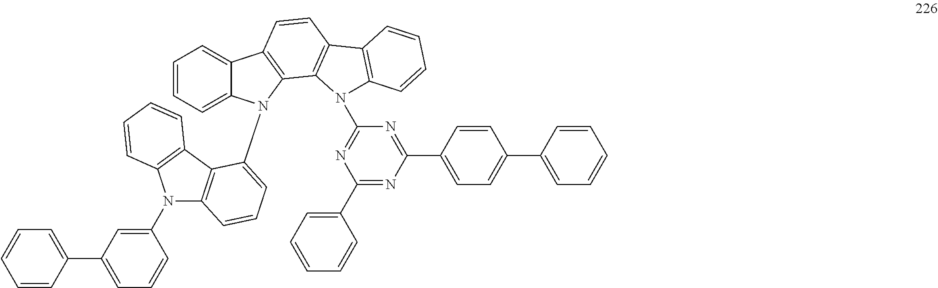 [SMILES: c1ccc(-c2ccc(-c3nc(-c4ccccc4)nc(-n4c5ccccc5c5ccc6c7ccccc7n(-c7cccc8c7c7ccccc7n8-c7cccc(-c8ccccc8)c7)c6c54)n3)cc2)cc1]

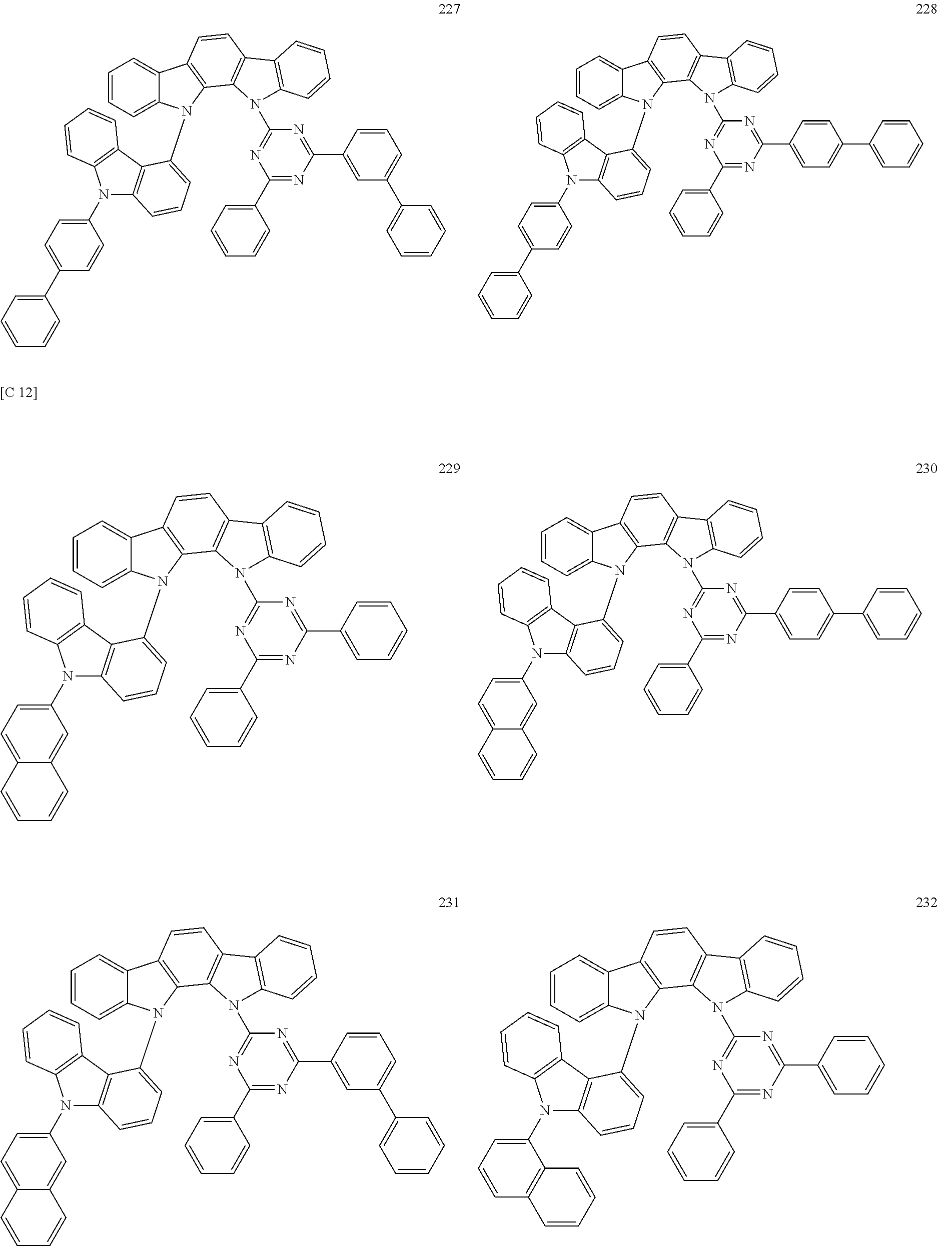

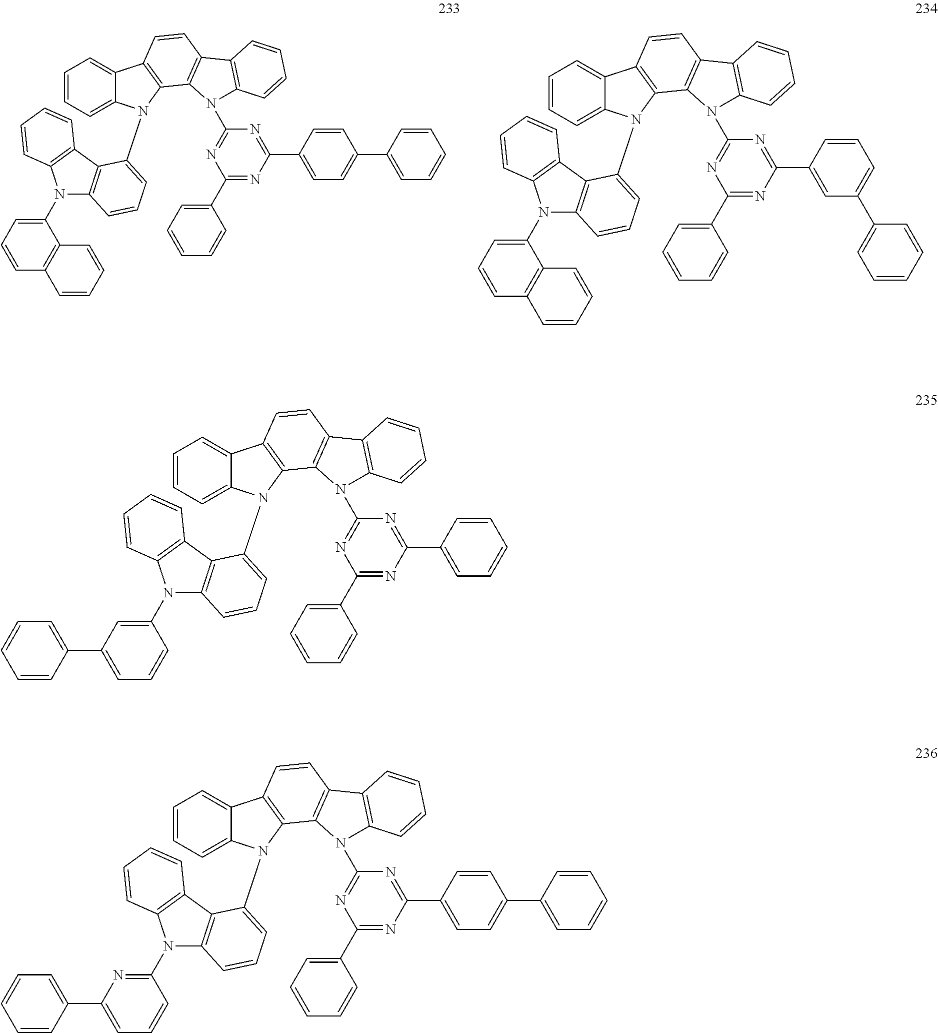 [SMILES: c1ccc(-c2ccc(-c3nc(-c4ccccc4)nc(-n4c5ccccc5c5ccc6c7ccccc7n(-c7cccc8c7c7ccccc7n8-c7cccc8ccccc78)c6c54)n3)cc2)cc1 ; c1ccc(-c2cccc(-c3nc(-c4ccccc4)nc(-n4c5ccccc5c5ccc6c7ccccc7n(-c7cccc8c7c7ccccc7n8-c7cccc8ccccc78)c6c54)n3)c2)cc1 ; c1ccc(-c2cccc(-n3c4ccccc4c4c(-n5c6ccccc6c6ccc7c8ccccc8n(-c8nc(-c9ccccc9)nc(-c9ccccc9)n8)c7c65)cccc43)c2)cc1 ; c1ccc(-c2ccc(-c3nc(-c4ccccc4)nc(-n4c5ccccc5c5ccc6c7ccccc7n(-c7cccc8c7c7ccccc7n8-c7cccc(-c8ccccc8)n7)c6c54)n3)cc2)cc1]

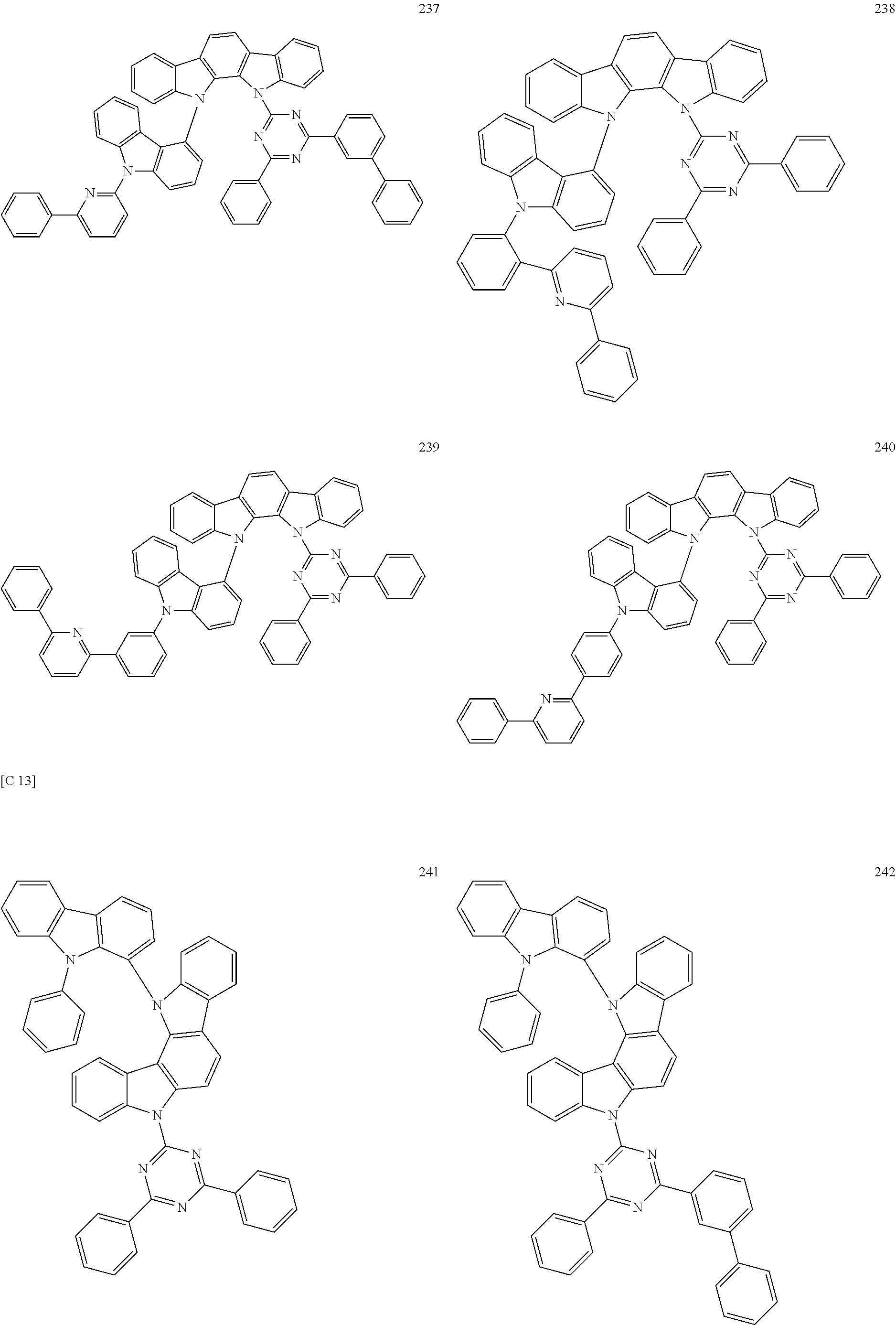

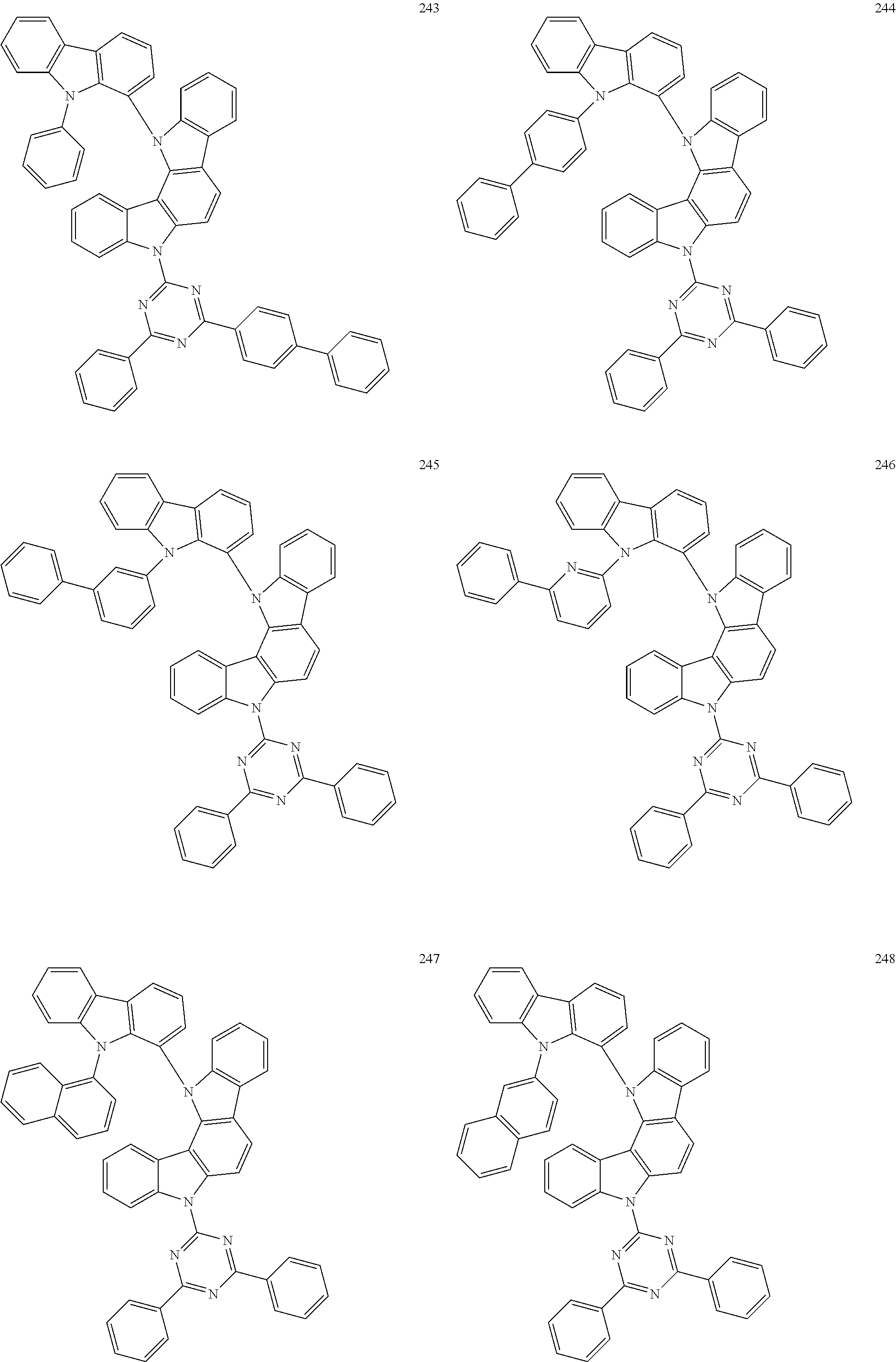

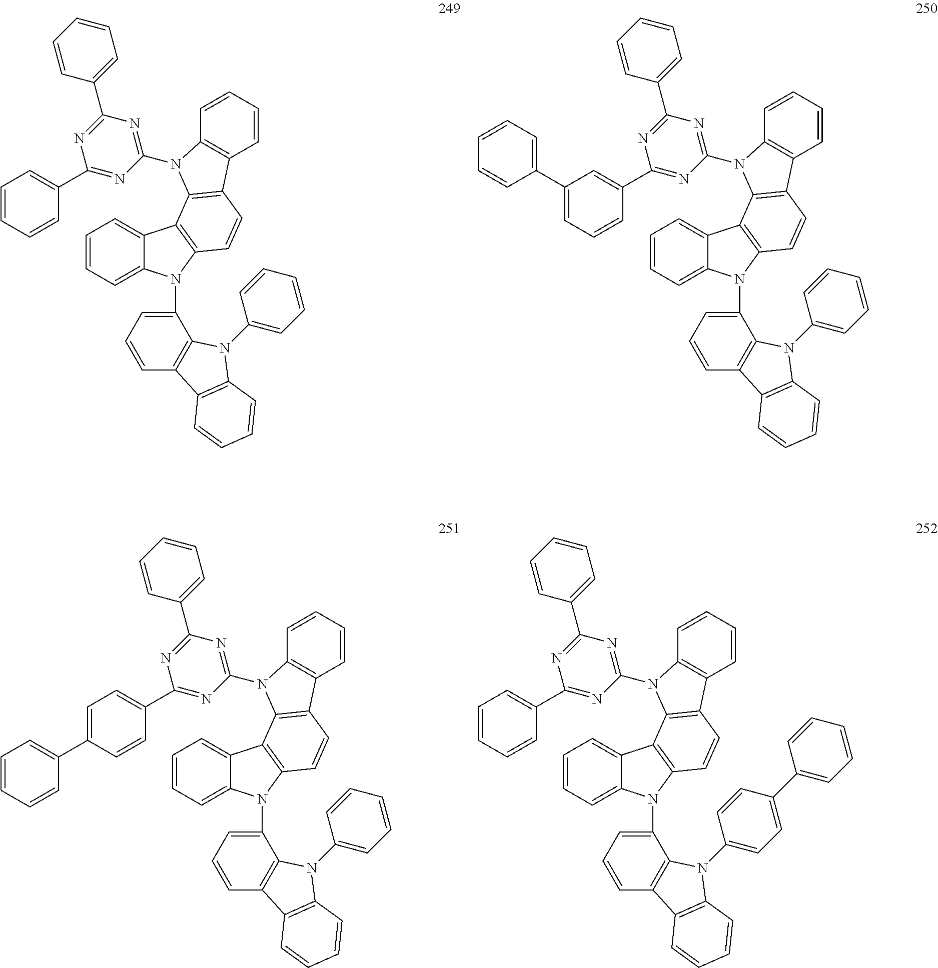 [SMILES: c1ccc(-c2nc(-c3ccccc3)nc(-n3c4ccccc4c4ccc5c(c6ccccc6n5-c5cccc6c7ccccc7n(-c7ccccc7)c56)c43)n2)cc1 ; c1ccc(-c2cccc(-c3nc(-c4ccccc4)nc(-n4c5ccccc5c5ccc6c(c7ccccc7n6-c6cccc7c8ccccc8n(-c8ccccc8)c67)c54)n3)c2)cc1 ; c1ccc(-c2ccc(-c3nc(-c4ccccc4)nc(-n4c5ccccc5c5ccc6c(c7ccccc7n6-c6cccc7c8ccccc8n(-c8ccccc8)c67)c54)n3)cc2)cc1 ; c1ccc(-c2ccc(-n3c4ccccc4c4cccc(-n5c6ccccc6c6c5ccc5c7ccccc7n(-c7nc(-c8ccccc8)nc(-c8ccccc8)n7)c56)c43)cc2)cc1]

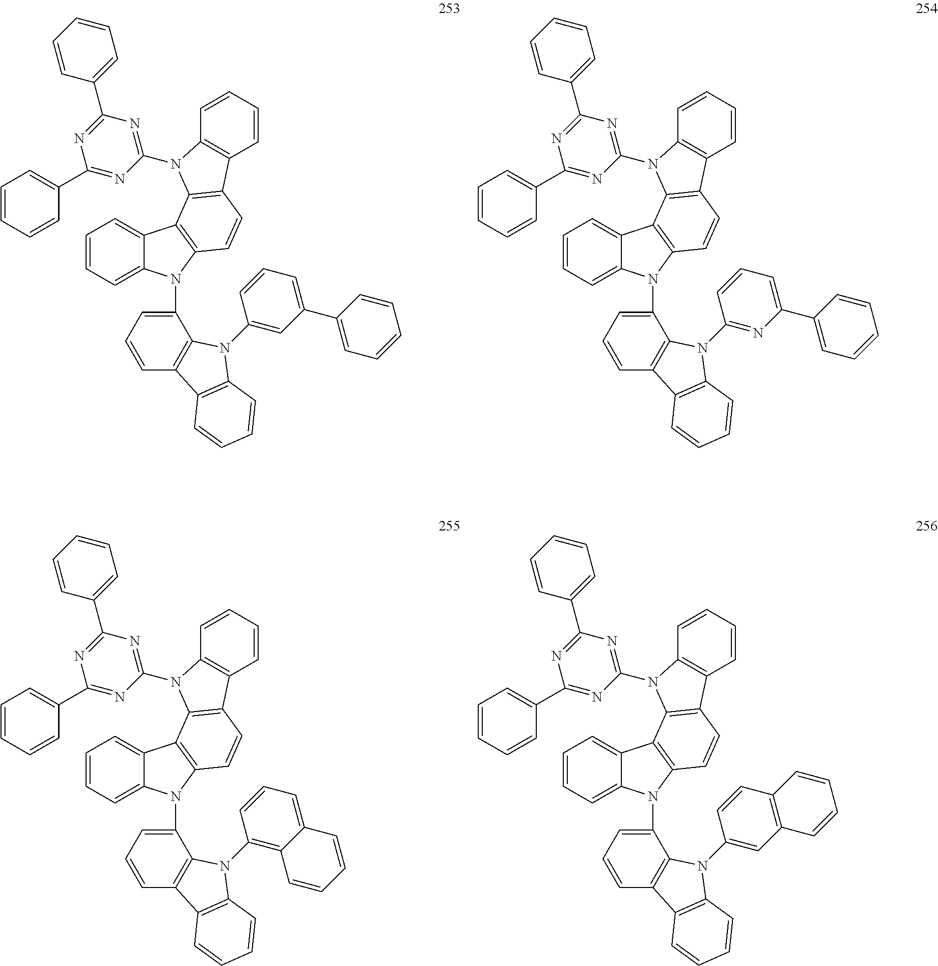 [SMILES: c1ccc(-c2cccc(-n3c4ccccc4c4cccc(-n5c6ccccc6c6c5ccc5c7ccccc7n(-c7nc(-c8ccccc8)nc(-c8ccccc8)n7)c56)c43)c2)cc1 ; c1ccc(-c2cccc(-n3c4ccccc4c4cccc(-n5c6ccccc6c6c5ccc5c7ccccc7n(-c7nc(-c8ccccc8)nc(-c8ccccc8)n7)c56)c43)n2)cc1 ; c1ccc(-c2nc(-c3ccccc3)nc(-n3c4ccccc4c4ccc5c(c6ccccc6n5-c5cccc6c7ccccc7n(-c7cccc8ccccc78)c56)c43)n2)cc1 ; c1ccc(-c2nc(-c3ccccc3)nc(-n3c4ccccc4c4ccc5c(c6ccccc6n5-c5cccc6c7ccccc7n(-c7ccc8ccccc8c7)c56)c43)n2)cc1]

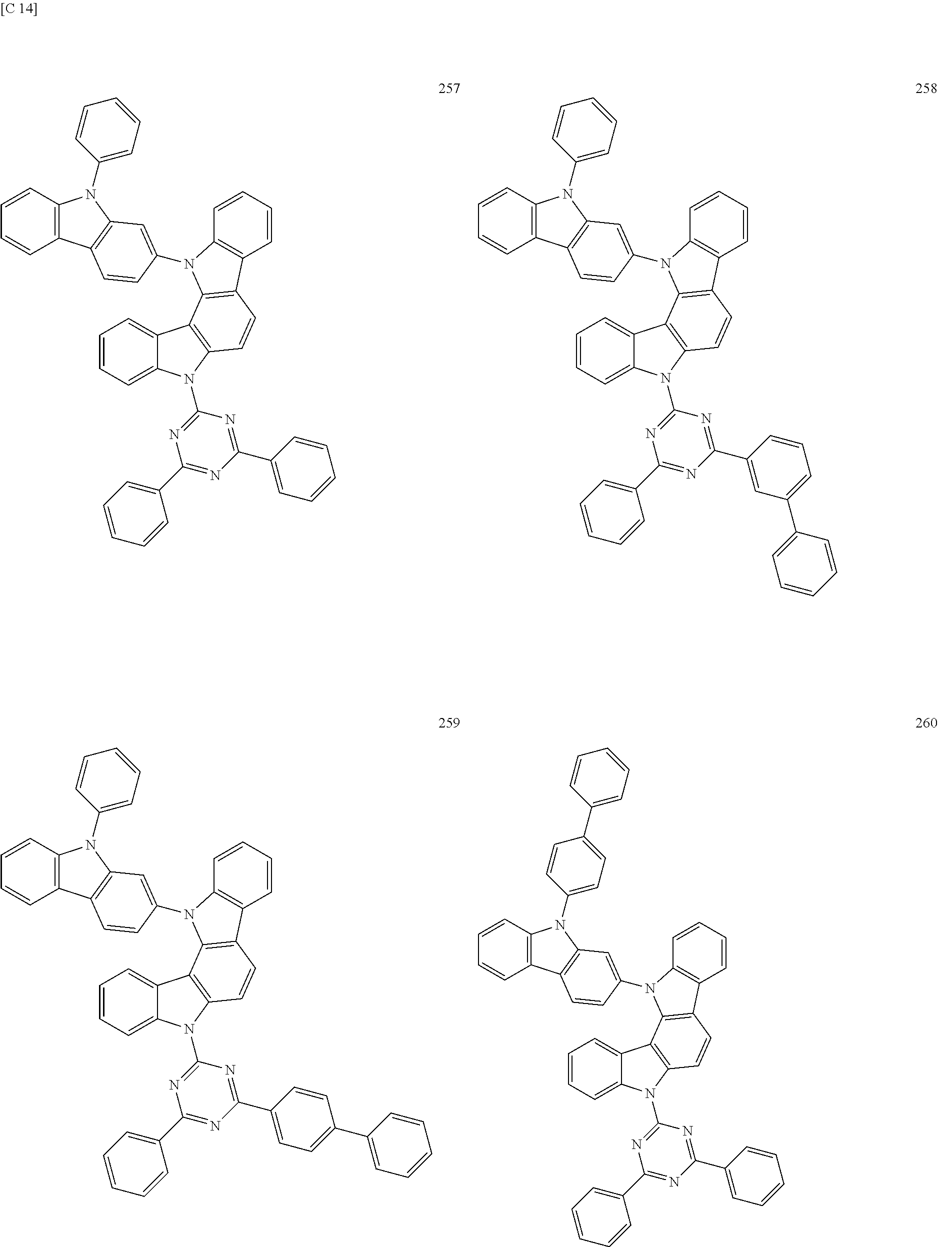 [SMILES: c1ccc(-c2nc(-c3ccccc3)nc(-n3c4ccccc4c4c3ccc3c5ccccc5n(-c5ccc6c7ccccc7n(-c7ccccc7)c6c5)c34)n2)cc1 ; c1ccc(-c2cccc(-c3nc(-c4ccccc4)nc(-n4c5ccccc5c5c4ccc4c6ccccc6n(-c6ccc7c8ccccc8n(-c8ccccc8)c7c6)c45)n3)c2)cc1 ; c1ccc(-c2ccc(-c3nc(-c4ccccc4)nc(-n4c5ccccc5c5c4ccc4c6ccccc6n(-c6ccc7c8ccccc8n(-c8ccccc8)c7c6)c45)n3)cc2)cc1 ; c1ccc(-c2ccc(-n3c4ccccc4c4ccc(-n5c6ccccc6c6ccc7c(c8ccccc8n7-c7nc(-c8ccccc8)nc(-c8ccccc8)n7)c65)cc43)cc2)cc1]

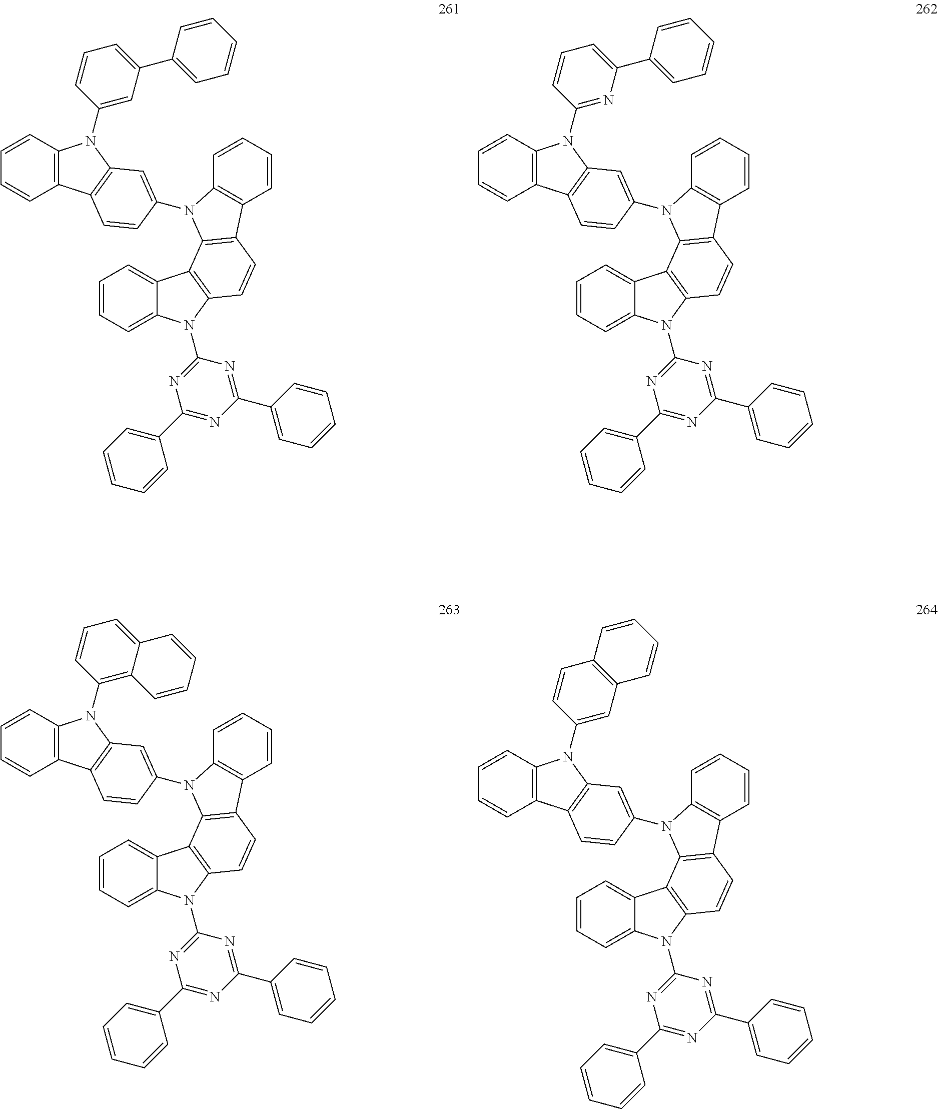 [SMILES: c1ccc(-c2cccc(-n3c4ccccc4c4ccc(-n5c6ccccc6c6ccc7c(c8ccccc8n7-c7nc(-c8ccccc8)nc(-c8ccccc8)n7)c65)cc43)c2)cc1 ; c1ccc(-c2cccc(-n3c4ccccc4c4ccc(-n5c6ccccc6c6ccc7c(c8ccccc8n7-c7nc(-c8ccccc8)nc(-c8ccccc8)n7)c65)cc43)n2)cc1 ; c1ccc(-c2nc(-c3ccccc3)nc(-n3c4ccccc4c4c3ccc3c5ccccc5n(-c5ccc6c7ccccc7n(-c7cccc8ccccc78)c6c5)c34)n2)cc1 ; c1ccc(-c2nc(-c3ccccc3)nc(-n3c4ccccc4c4c3ccc3c5ccccc5n(-c5ccc6c7ccccc7n(-c7ccc8ccccc8c7)c6c5)c34)n2)cc1]

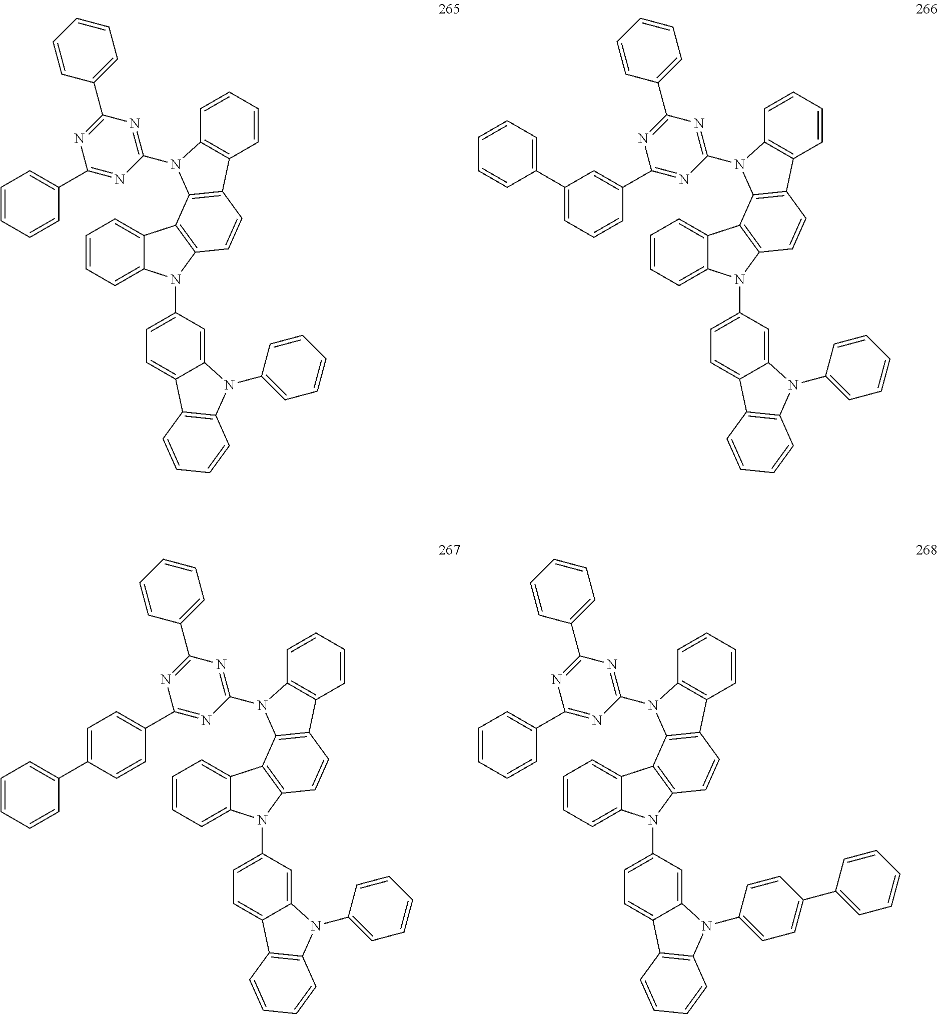 [SMILES: c1ccc(-c2nc(-c3ccccc3)nc(-n3c4ccccc4c4ccc5c(c6ccccc6n5-c5ccc6c7ccccc7n(-c7ccccc7)c6c5)c43)n2)cc1 ; c1ccc(-c2cccc(-c3nc(-c4ccccc4)nc(-n4c5ccccc5c5ccc6c(c7ccccc7n6-c6ccc7c8ccccc8n(-c8ccccc8)c7c6)c54)n3)c2)cc1 ; c1ccc(-c2ccc(-c3nc(-c4ccccc4)nc(-n4c5ccccc5c5ccc6c(c7ccccc7n6-c6ccc7c8ccccc8n(-c8ccccc8)c7c6)c54)n3)cc2)cc1 ; c1ccc(-c2ccc(-n3c4ccccc4c4ccc(-n5c6ccccc6c6c5ccc5c7ccccc7n(-c7nc(-c8ccccc8)nc(-c8ccccc8)n7)c56)cc43)cc2)cc1]

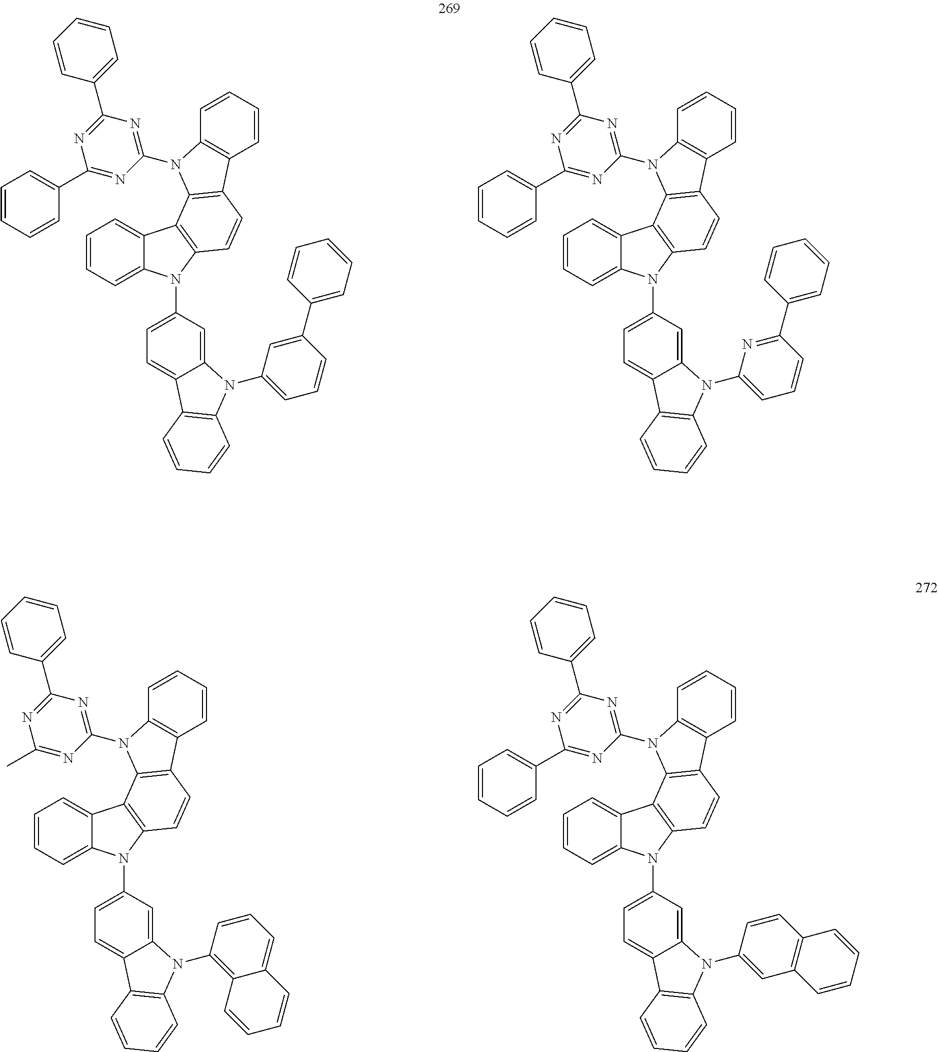 [SMILES: c1ccc(-c2cccc(-n3c4ccccc4c4ccc(-n5c6ccccc6c6c5ccc5c7ccccc7n(-c7nc(-c8ccccc8)nc(-c8ccccc8)n7)c56)cc43)c2)cc1 ; c1ccc(-c2cccc(-n3c4ccccc4c4ccc(-n5c6ccccc6c6c5ccc5c7ccccc7n(-c7nc(-c8ccccc8)nc(-c8ccccc8)n7)c56)cc43)n2)cc1 ; Cc1nc(-c2ccccc2)nc(-n2c3ccccc3c3ccc4c(c5ccccc5n4-c4ccc5c6ccccc6n(-c6cccc7ccccc67)c5c4)c32)n1 ; c1ccc(-c2nc(-c3ccccc3)nc(-n3c4ccccc4c4ccc5c(c6ccccc6n5-c5ccc6c7ccccc7n(-c7ccc8ccccc8c7)c6c5)c43)n2)cc1]

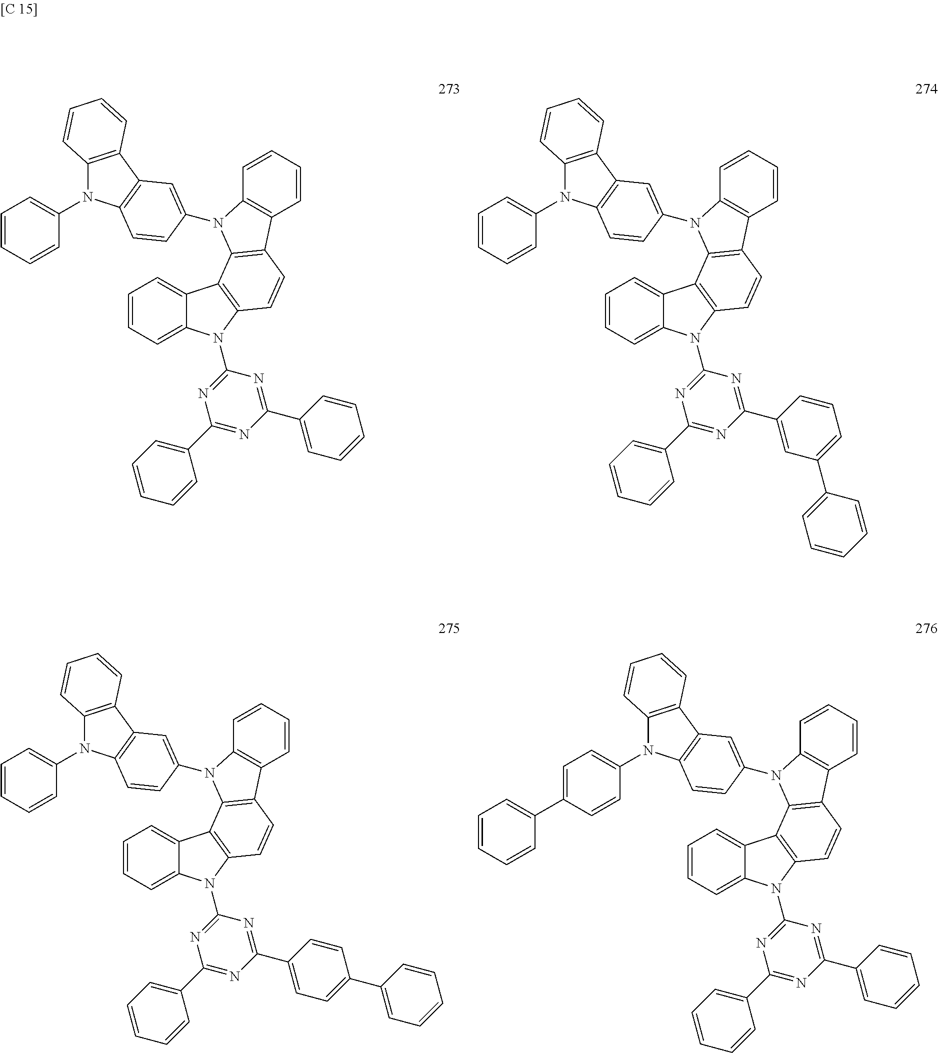 [SMILES: c1ccc(-c2nc(-c3ccccc3)nc(-n3c4ccccc4c4c3ccc3c5ccccc5n(-c5ccc6c(c5)c5ccccc5n6-c5ccccc5)c34)n2)cc1 ; c1ccc(-c2cccc(-c3nc(-c4ccccc4)nc(-n4c5ccccc5c5c4ccc4c6ccccc6n(-c6ccc7c(c6)c6ccccc6n7-c6ccccc6)c45)n3)c2)cc1 ; c1ccc(-c2ccc(-c3nc(-c4ccccc4)nc(-n4c5ccccc5c5c4ccc4c6ccccc6n(-c6ccc7c(c6)c6ccccc6n7-c6ccccc6)c45)n3)cc2)cc1 ; c1ccc(-c2ccc(-n3c4ccccc4c4cc(-n5c6ccccc6c6ccc7c(c8ccccc8n7-c7nc(-c8ccccc8)nc(-c8ccccc8)n7)c65)ccc43)cc2)cc1]

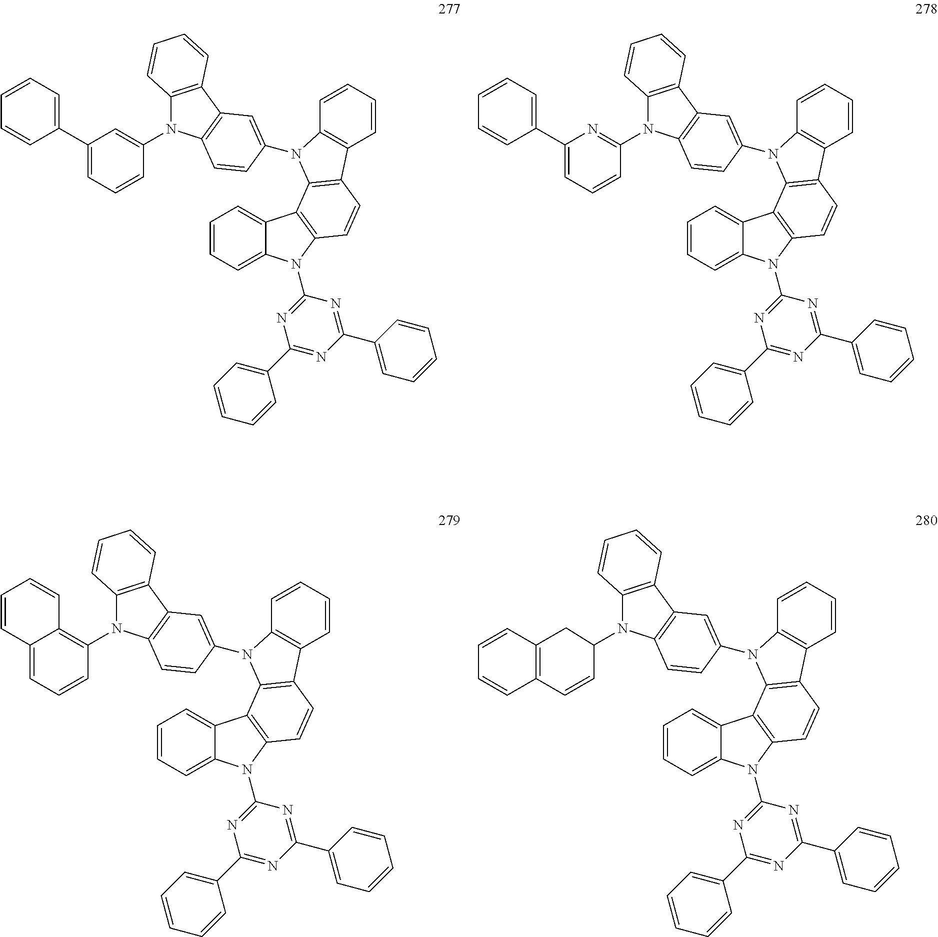 [SMILES: c1ccc(-c2cccc(-n3c4ccccc4c4cc(-n5c6ccccc6c6ccc7c(c8ccccc8n7-c7nc(-c8ccccc8)nc(-c8ccccc8)n7)c65)ccc43)c2)cc1 ; c1ccc(-c2cccc(-n3c4ccccc4c4cc(-n5c6ccccc6c6ccc7c(c8ccccc8n7-c7nc(-c8ccccc8)nc(-c8ccccc8)n7)c65)ccc43)n2)cc1 ; c1ccc(-c2nc(-c3ccccc3)nc(-n3c4ccccc4c4c3ccc3c5ccccc5n(-c5ccc6c(c5)c5ccccc5n6-c5cccc6ccccc56)c34)n2)cc1 ; C1=CC(n2c3ccccc3c3cc(-n4c5ccccc5c5ccc6c(c7ccccc7n6-c6nc(-c7ccccc7)nc(-c7ccccc7)n6)c54)ccc32)Cc2ccccc21]

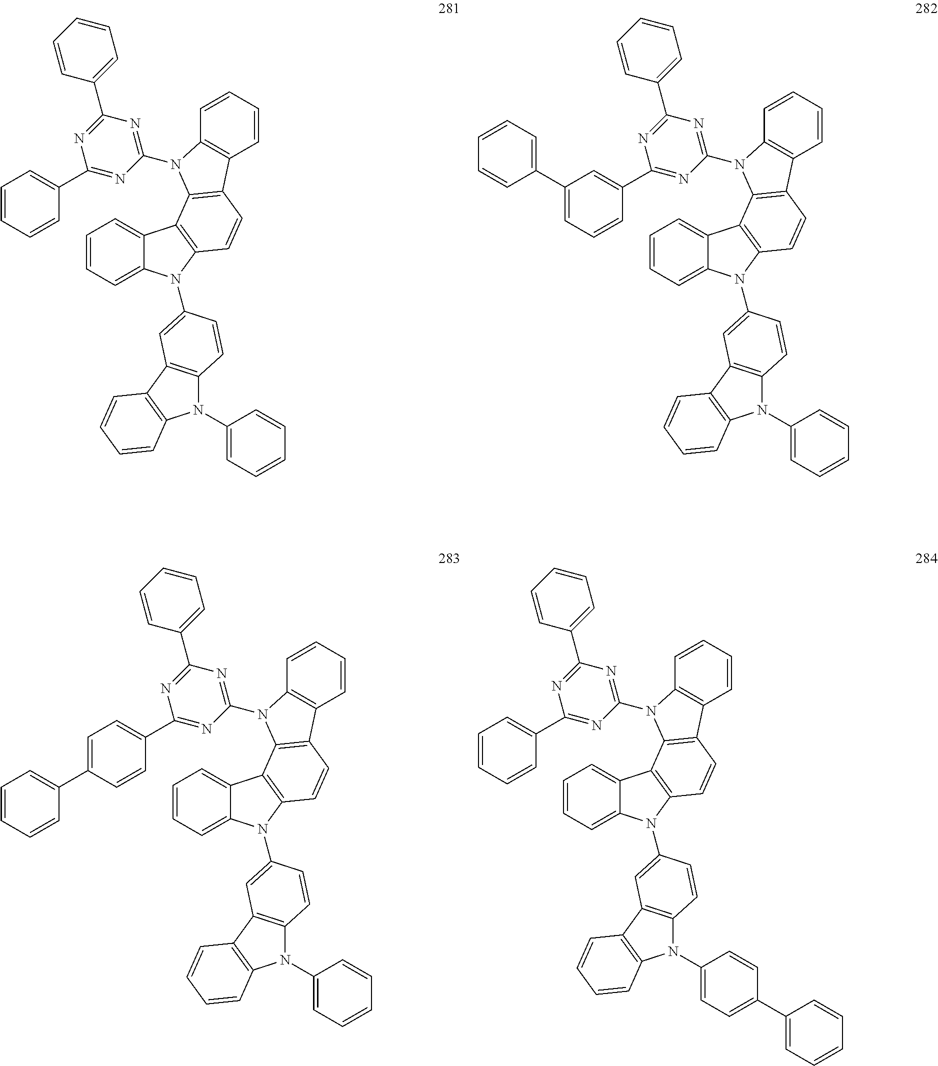 [SMILES: c1ccc(-c2nc(-c3ccccc3)nc(-n3c4ccccc4c4ccc5c(c6ccccc6n5-c5ccc6c(c5)c5ccccc5n6-c5ccccc5)c43)n2)cc1 ; c1ccc(-c2cccc(-c3nc(-c4ccccc4)nc(-n4c5ccccc5c5ccc6c(c7ccccc7n6-c6ccc7c(c6)c6ccccc6n7-c6ccccc6)c54)n3)c2)cc1 ; c1ccc(-c2ccc(-c3nc(-c4ccccc4)nc(-n4c5ccccc5c5ccc6c(c7ccccc7n6-c6ccc7c(c6)c6ccccc6n7-c6ccccc6)c54)n3)cc2)cc1 ; c1ccc(-c2ccc(-n3c4ccccc4c4cc(-n5c6ccccc6c6c5ccc5c7ccccc7n(-c7nc(-c8ccccc8)nc(-c8ccccc8)n7)c56)ccc43)cc2)cc1]

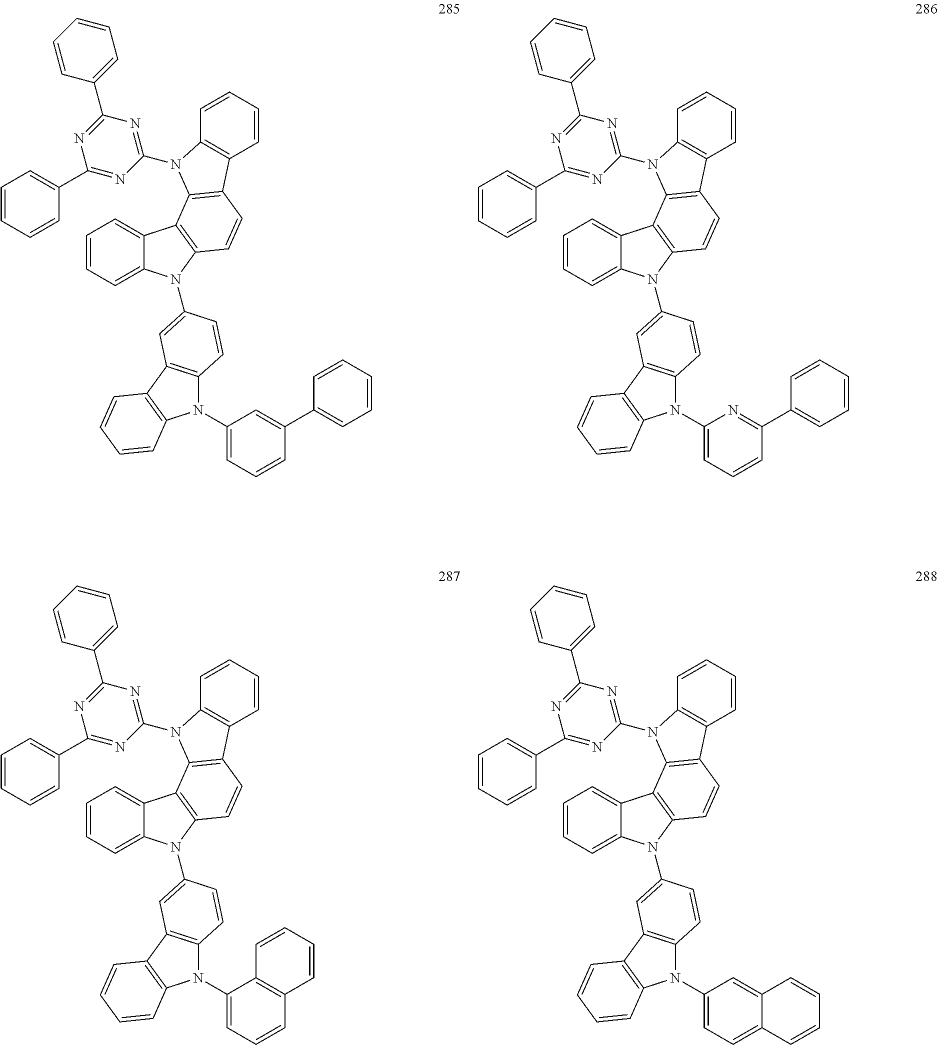 [SMILES: c1ccc(-c2cccc(-n3c4ccccc4c4cc(-n5c6ccccc6c6c5ccc5c7ccccc7n(-c7nc(-c8ccccc8)nc(-c8ccccc8)n7)c56)ccc43)c2)cc1 ; c1ccc(-c2cccc(-n3c4ccccc4c4cc(-n5c6ccccc6c6c5ccc5c7ccccc7n(-c7nc(-c8ccccc8)nc(-c8ccccc8)n7)c56)ccc43)n2)cc1 ; c1ccc(-c2nc(-c3ccccc3)nc(-n3c4ccccc4c4ccc5c(c6ccccc6n5-c5ccc6c(c5)c5ccccc5n6-c5cccc6ccccc56)c43)n2)cc1 ; c1ccc(-c2nc(-c3ccccc3)nc(-n3c4ccccc4c4ccc5c(c6ccccc6n5-c5ccc6c(c5)c5ccccc5n6-c5ccc6ccccc6c5)c43)n2)cc1]

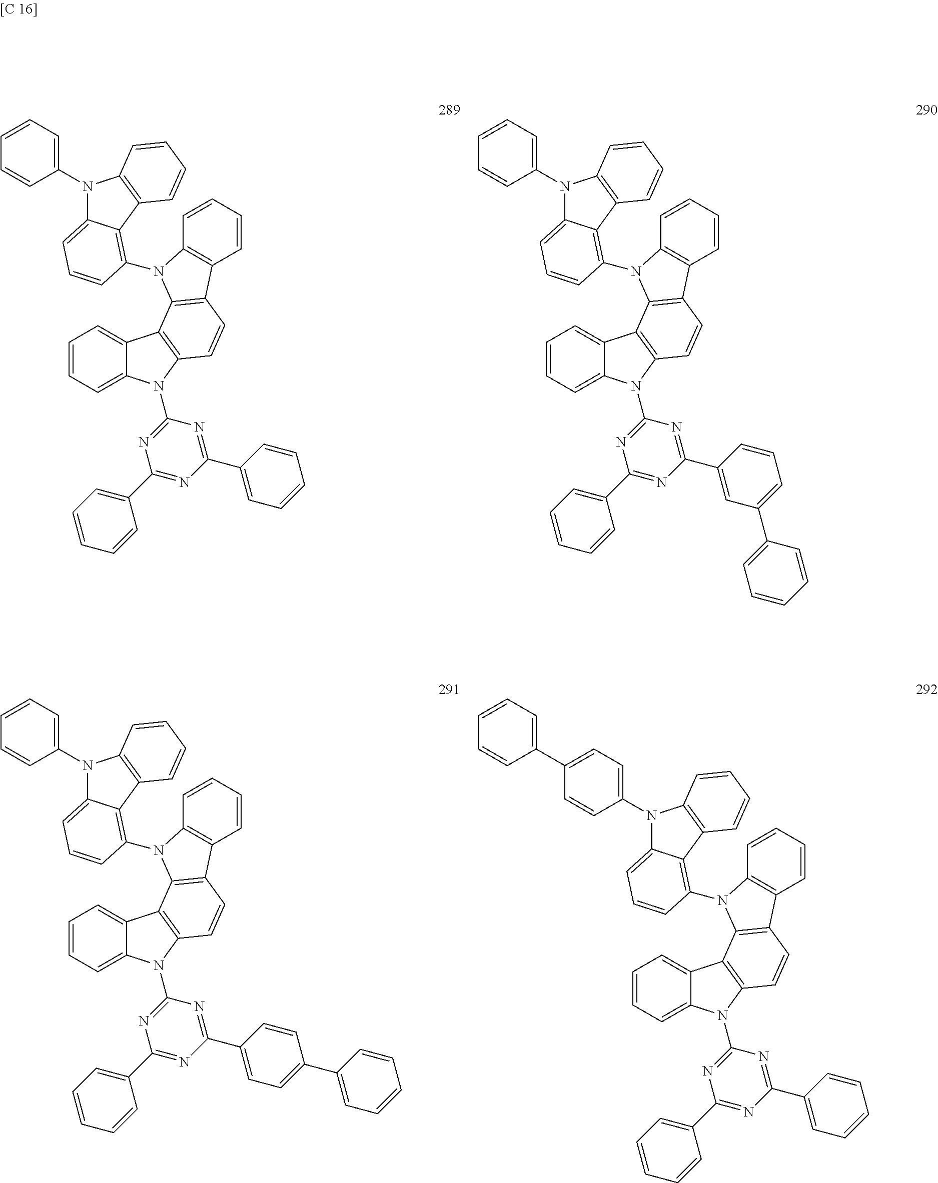 [SMILES: c1ccc(-c2nc(-c3ccccc3)nc(-n3c4ccccc4c4c3ccc3c5ccccc5n(-c5cccc6c5c5ccccc5n6-c5ccccc5)c34)n2)cc1 ; c1ccc(-c2cccc(-c3nc(-c4ccccc4)nc(-n4c5ccccc5c5c4ccc4c6ccccc6n(-c6cccc7c6c6ccccc6n7-c6ccccc6)c45)n3)c2)cc1 ; c1ccc(-c2ccc(-c3nc(-c4ccccc4)nc(-n4c5ccccc5c5c4ccc4c6ccccc6n(-c6cccc7c6c6ccccc6n7-c6ccccc6)c45)n3)cc2)cc1 ; c1ccc(-c2ccc(-n3c4ccccc4c4c(-n5c6ccccc6c6ccc7c(c8ccccc8n7-c7nc(-c8ccccc8)nc(-c8ccccc8)n7)c65)cccc43)cc2)cc1]

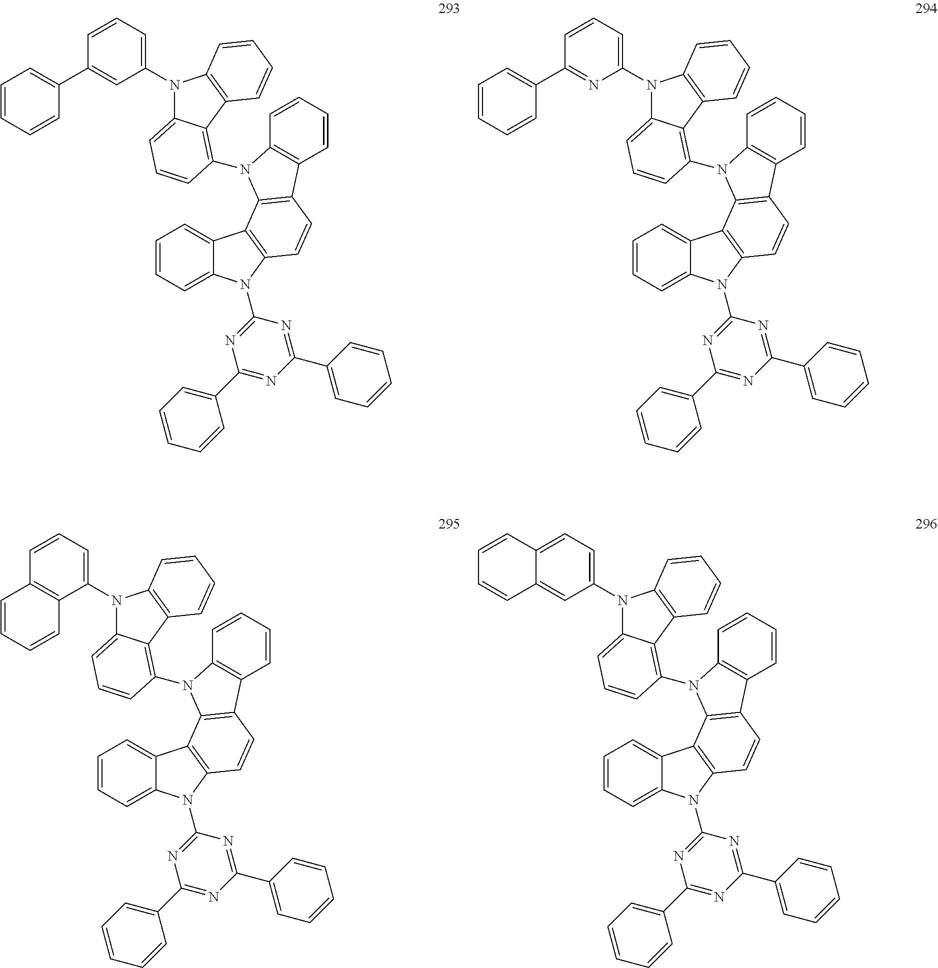 [SMILES: c1ccc(-c2cccc(-n3c4ccccc4c4c(-n5c6ccccc6c6ccc7c(c8ccccc8n7-c7nc(-c8ccccc8)nc(-c8ccccc8)n7)c65)cccc43)c2)cc1 ; c1ccc(-c2cccc(-n3c4ccccc4c4c(-n5c6ccccc6c6ccc7c(c8ccccc8n7-c7nc(-c8ccccc8)nc(-c8ccccc8)n7)c65)cccc43)n2)cc1 ; c1ccc(-c2nc(-c3ccccc3)nc(-n3c4ccccc4c4c3ccc3c5ccccc5n(-c5cccc6c5c5ccccc5n6-c5cccc6ccccc56)c34)n2)cc1 ; c1ccc(-c2nc(-c3ccccc3)nc(-n3c4ccccc4c4c3ccc3c5ccccc5n(-c5cccc6c5c5ccccc5n6-c5ccc6ccccc6c5)c34)n2)cc1]

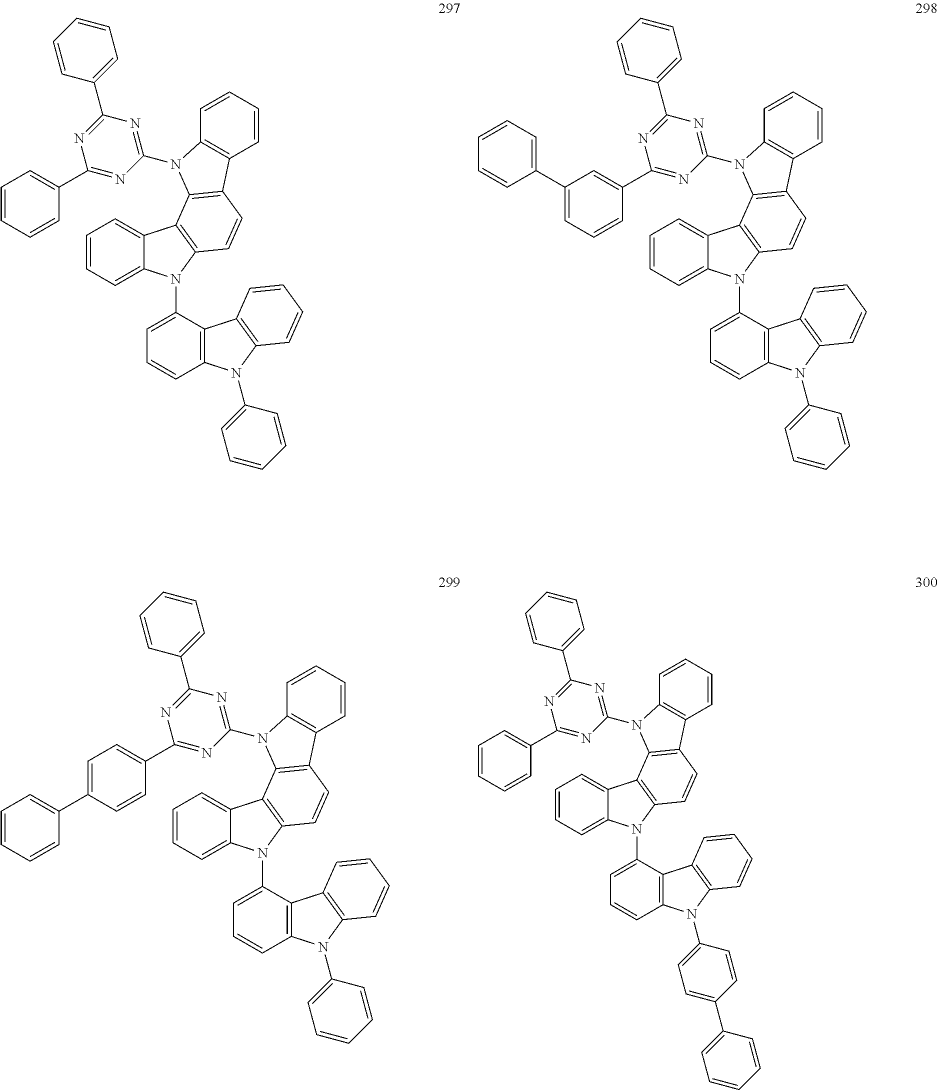 [SMILES: c1ccc(-c2nc(-c3ccccc3)nc(-n3c4ccccc4c4ccc5c(c6ccccc6n5-c5cccc6c5c5ccccc5n6-c5ccccc5)c43)n2)cc1 ; c1ccc(-c2cccc(-c3nc(-c4ccccc4)nc(-n4c5ccccc5c5ccc6c(c7ccccc7n6-c6cccc7c6c6ccccc6n7-c6ccccc6)c54)n3)c2)cc1 ; c1ccc(-c2ccc(-c3nc(-c4ccccc4)nc(-n4c5ccccc5c5ccc6c(c7ccccc7n6-c6cccc7c6c6ccccc6n7-c6ccccc6)c54)n3)cc2)cc1 ; c1ccc(-c2ccc(-n3c4ccccc4c4c(-n5c6ccccc6c6c5ccc5c7ccccc7n(-c7nc(-c8ccccc8)nc(-c8ccccc8)n7)c56)cccc43)cc2)cc1]

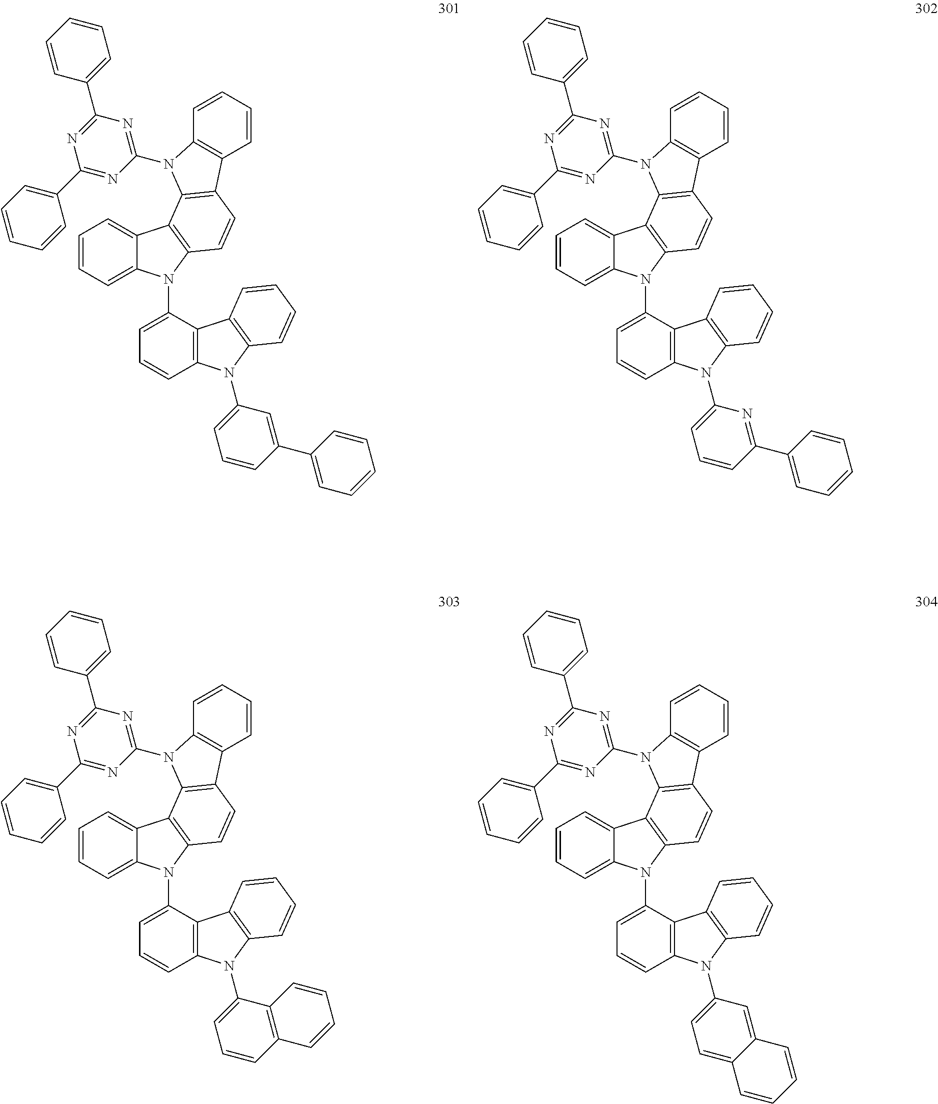 [SMILES: c1ccc(-c2cccc(-n3c4ccccc4c4c(-n5c6ccccc6c6c5ccc5c7ccccc7n(-c7nc(-c8ccccc8)nc(-c8ccccc8)n7)c56)cccc43)c2)cc1 ; c1ccc(-c2cccc(-n3c4ccccc4c4c(-n5c6ccccc6c6c5ccc5c7ccccc7n(-c7nc(-c8ccccc8)nc(-c8ccccc8)n7)c56)cccc43)n2)cc1 ; c1ccc(-c2nc(-c3ccccc3)nc(-n3c4ccccc4c4ccc5c(c6ccccc6n5-c5cccc6c5c5ccccc5n6-c5cccc6ccccc56)c43)n2)cc1 ; c1ccc(-c2nc(-c3ccccc3)nc(-n3c4ccccc4c4ccc5c(c6ccccc6n5-c5cccc6c5c5ccccc5n6-c5ccc6ccccc6c5)c43)n2)cc1]

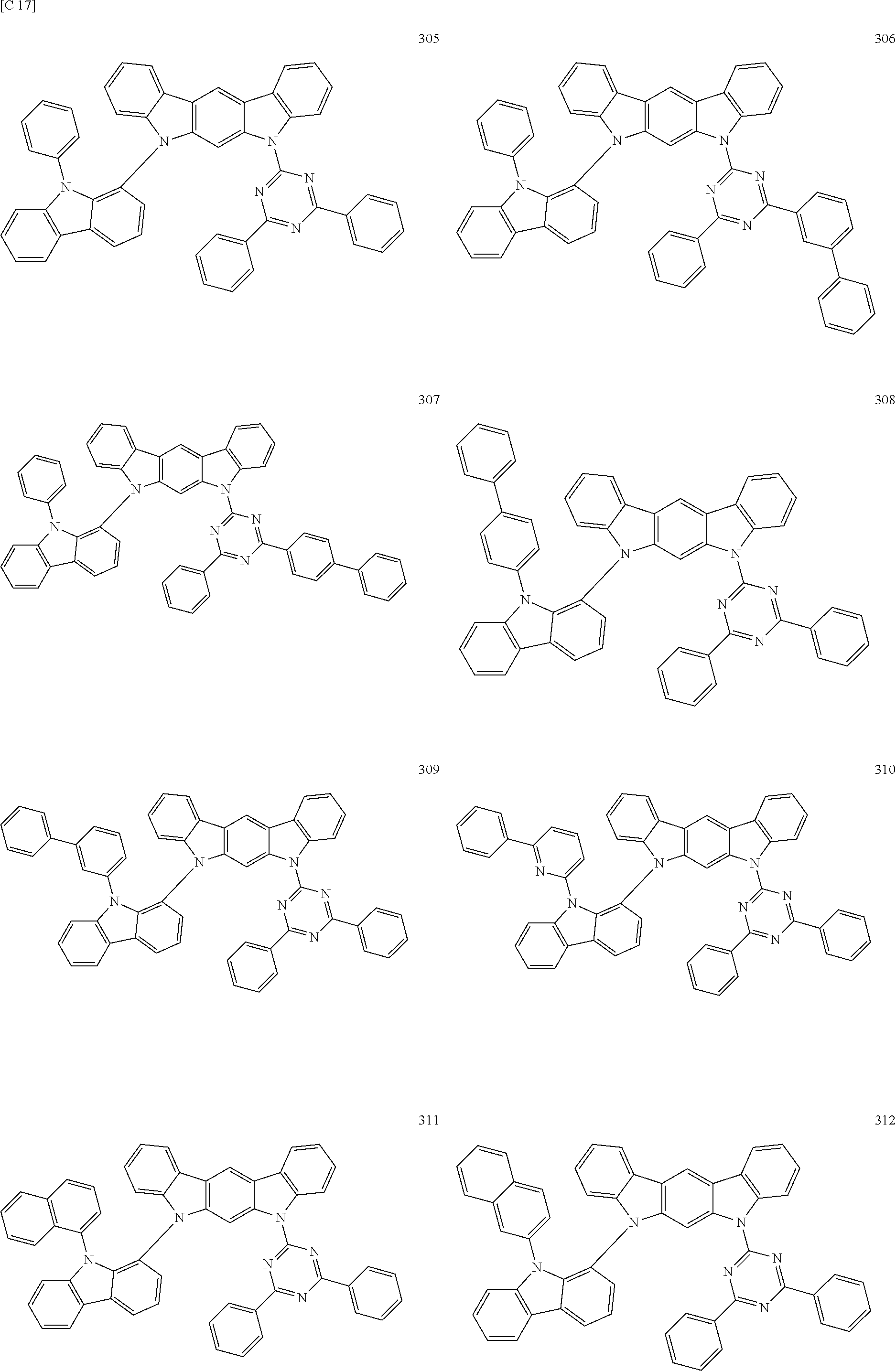

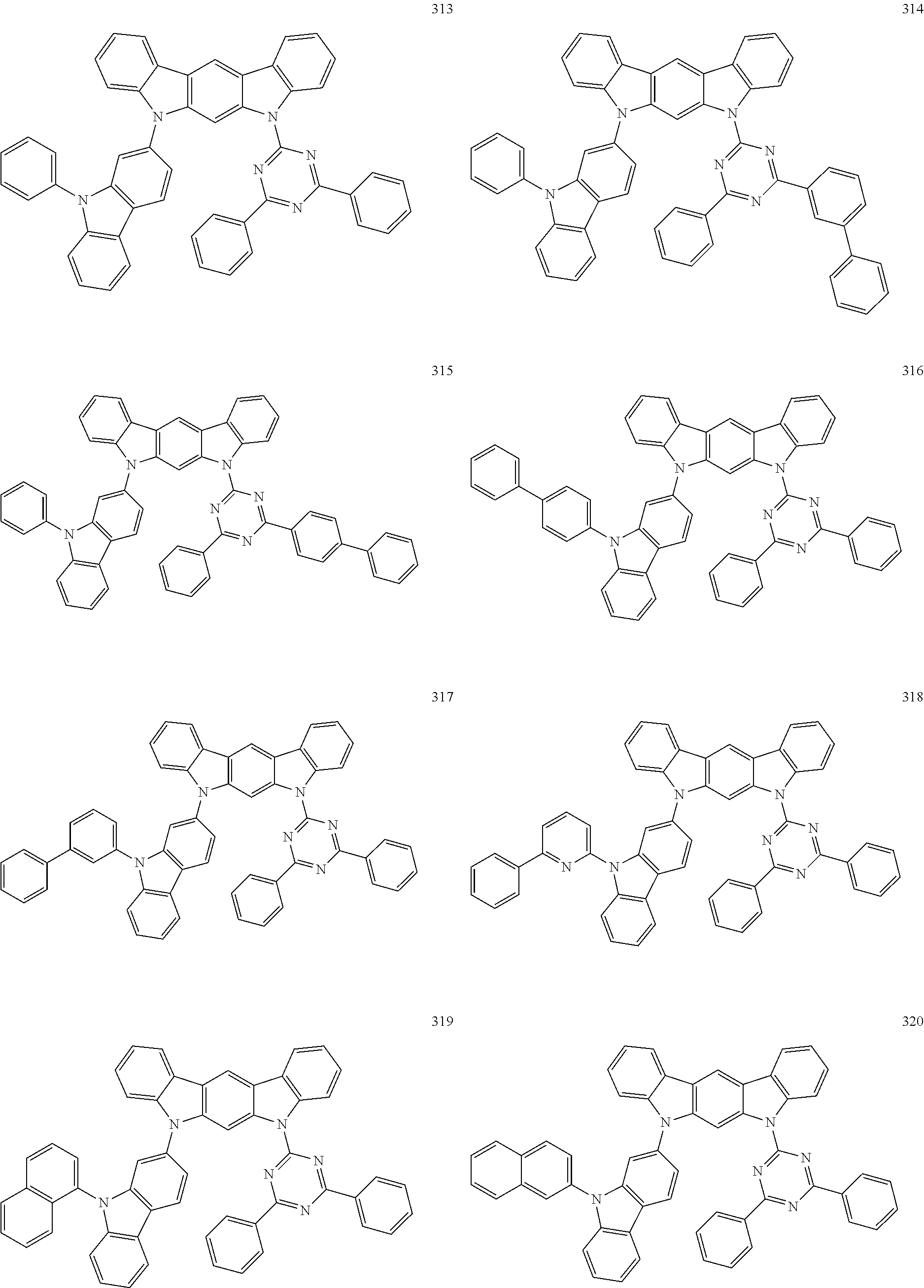

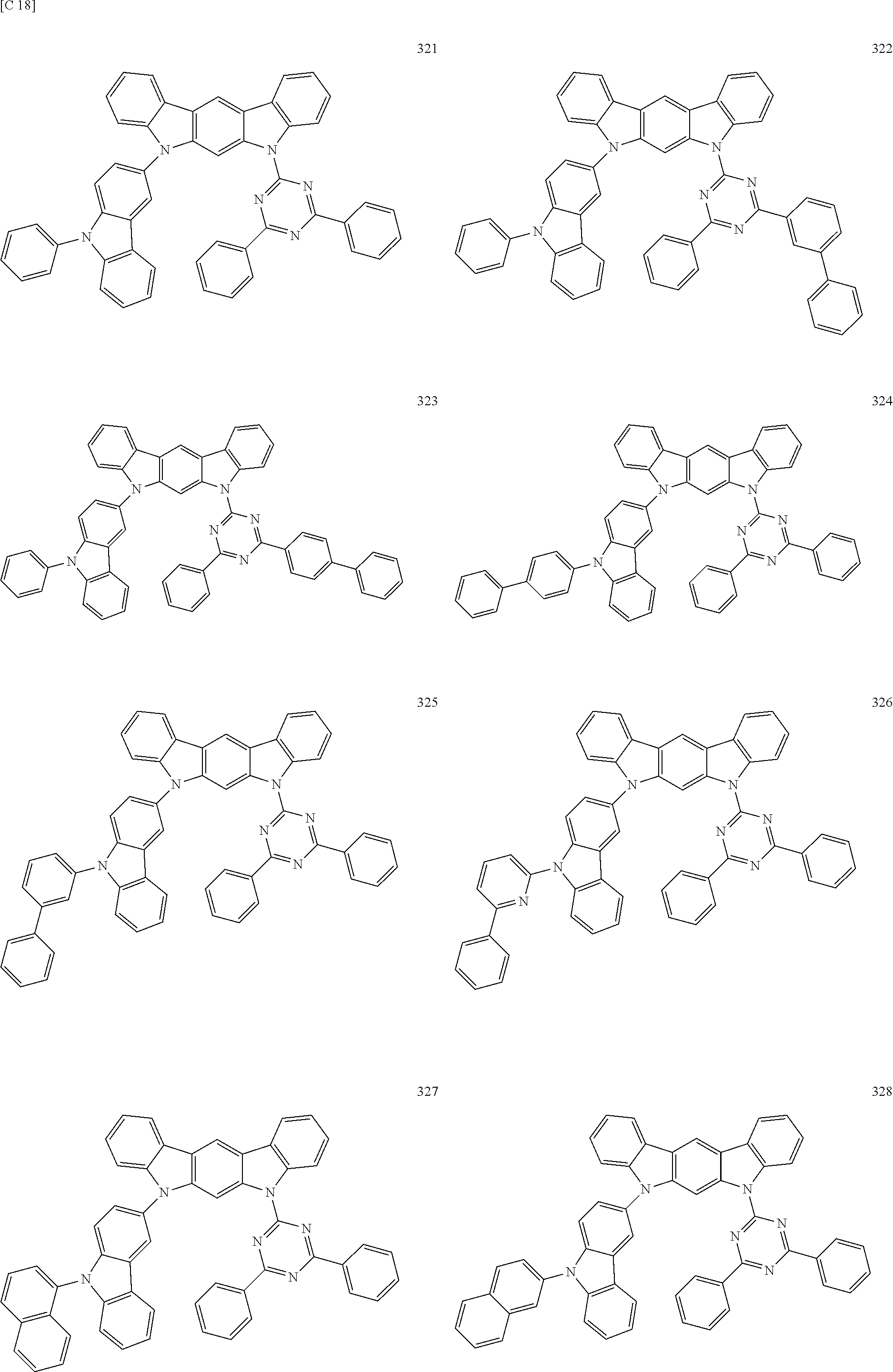

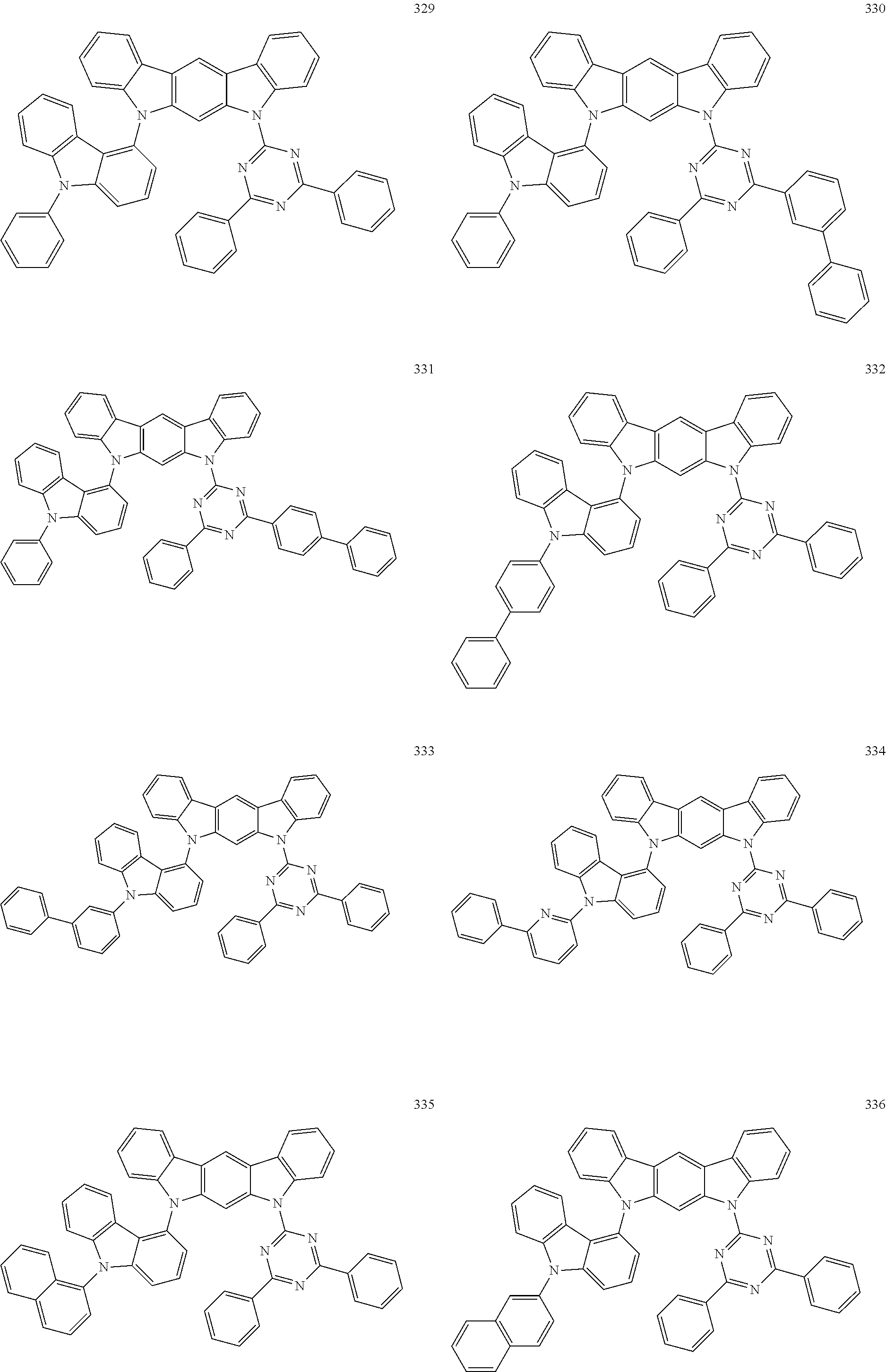

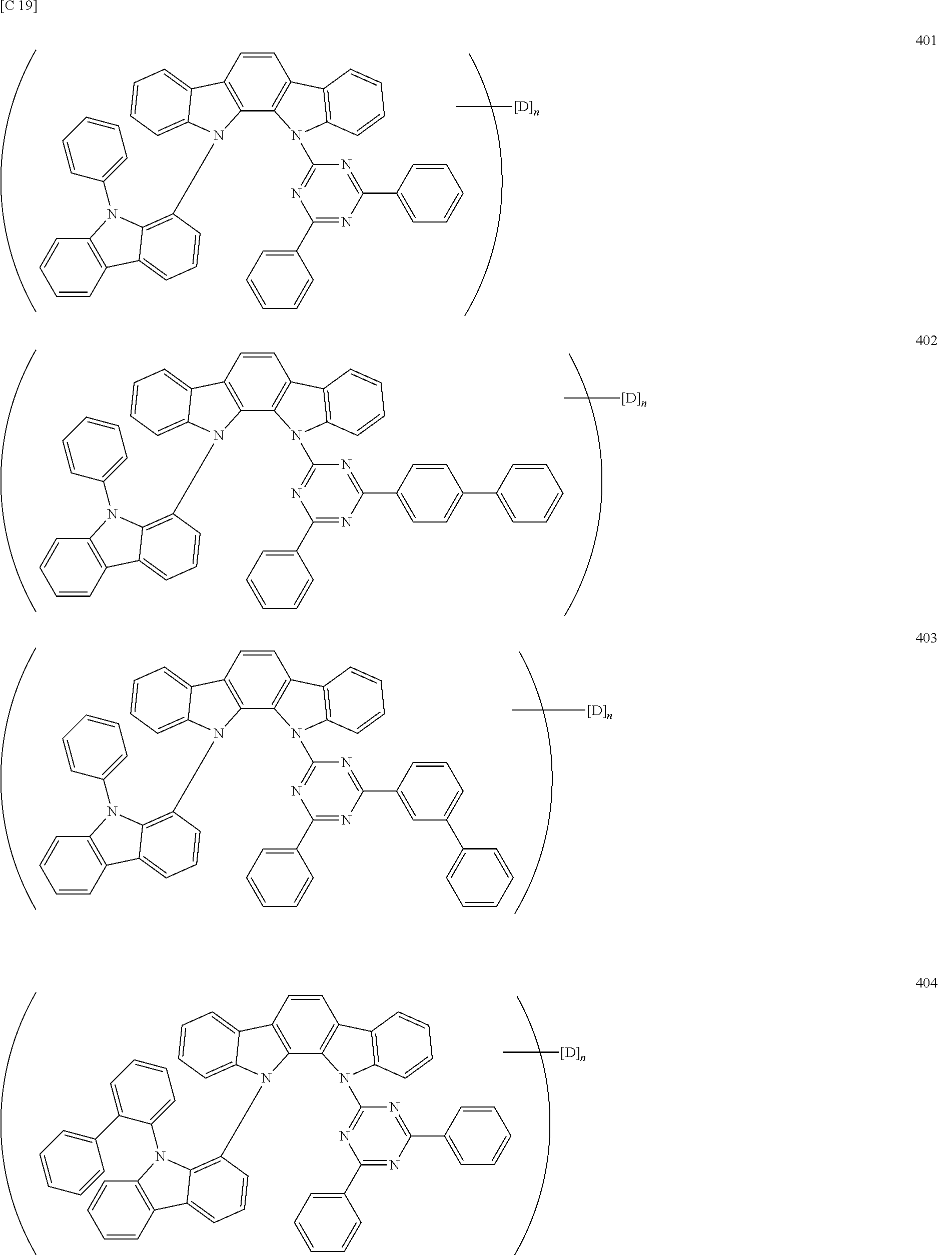

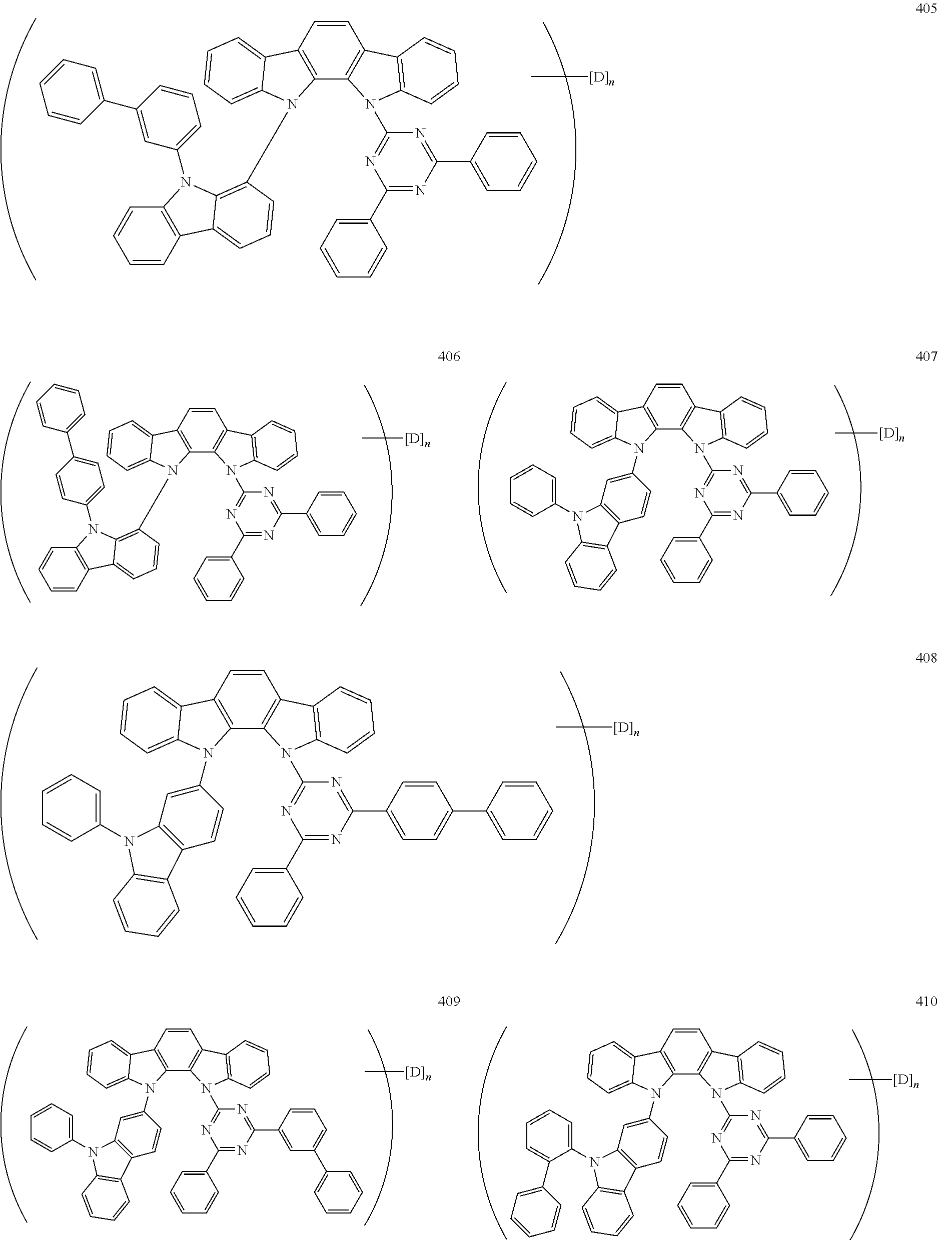

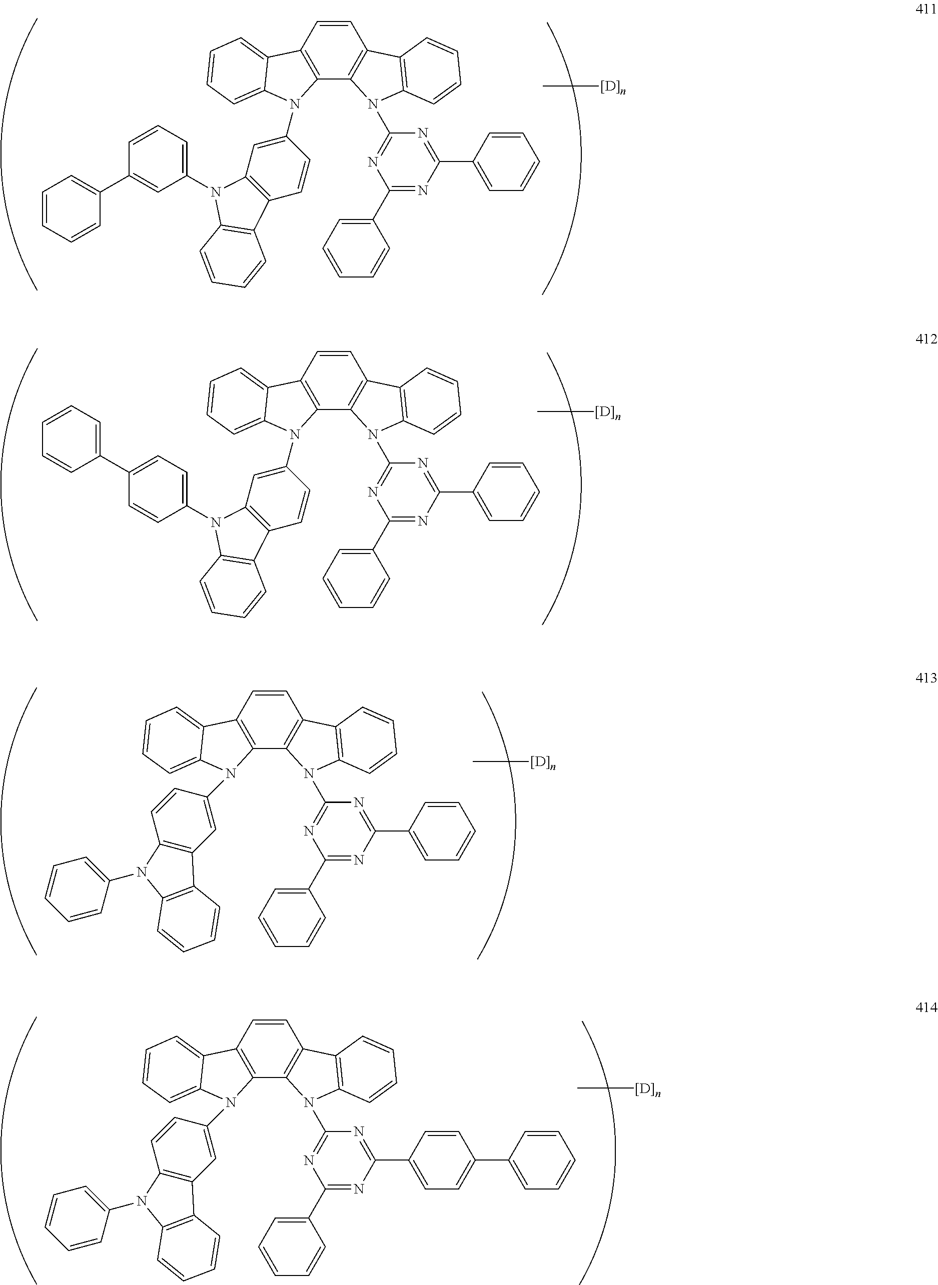

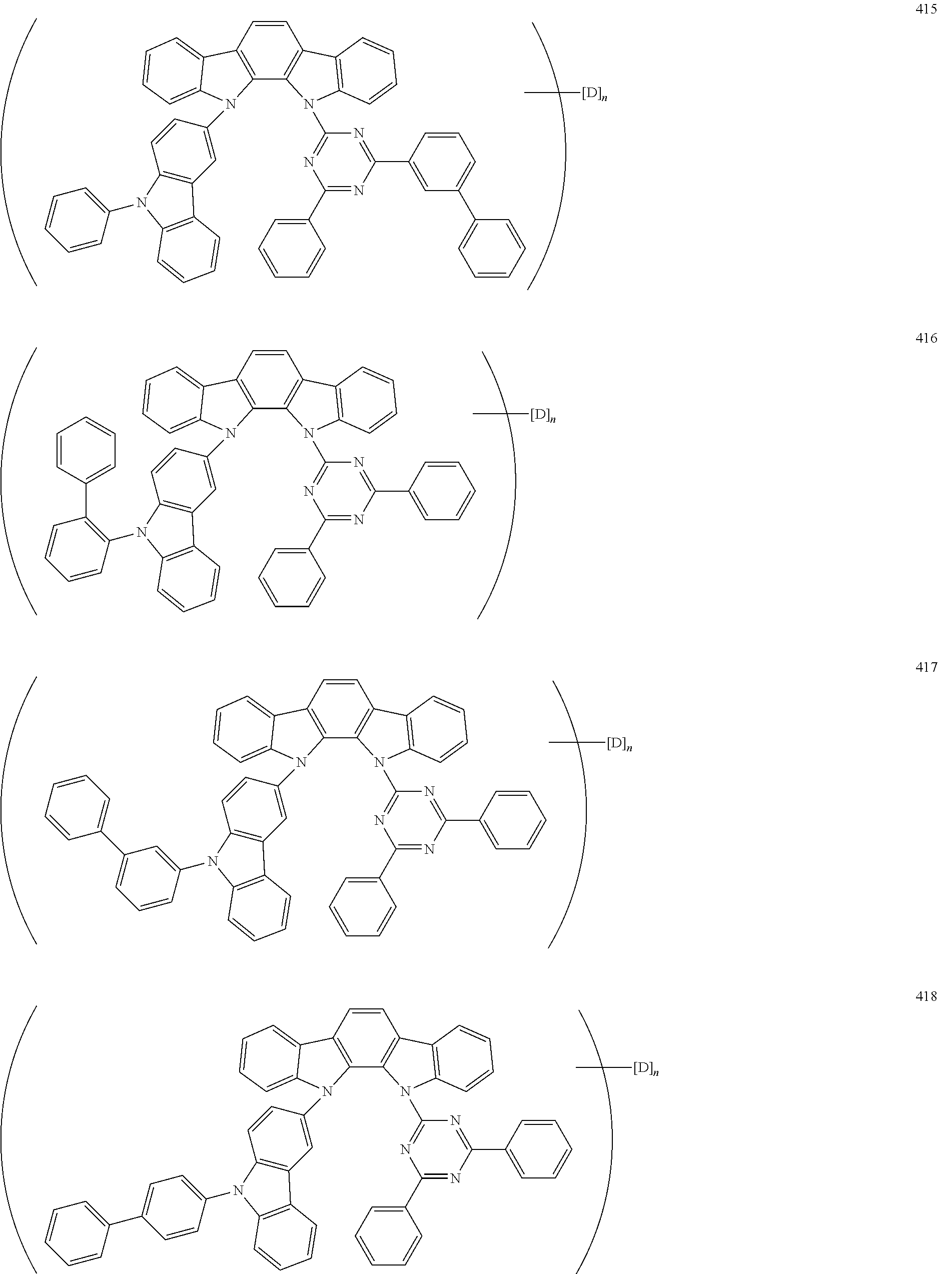

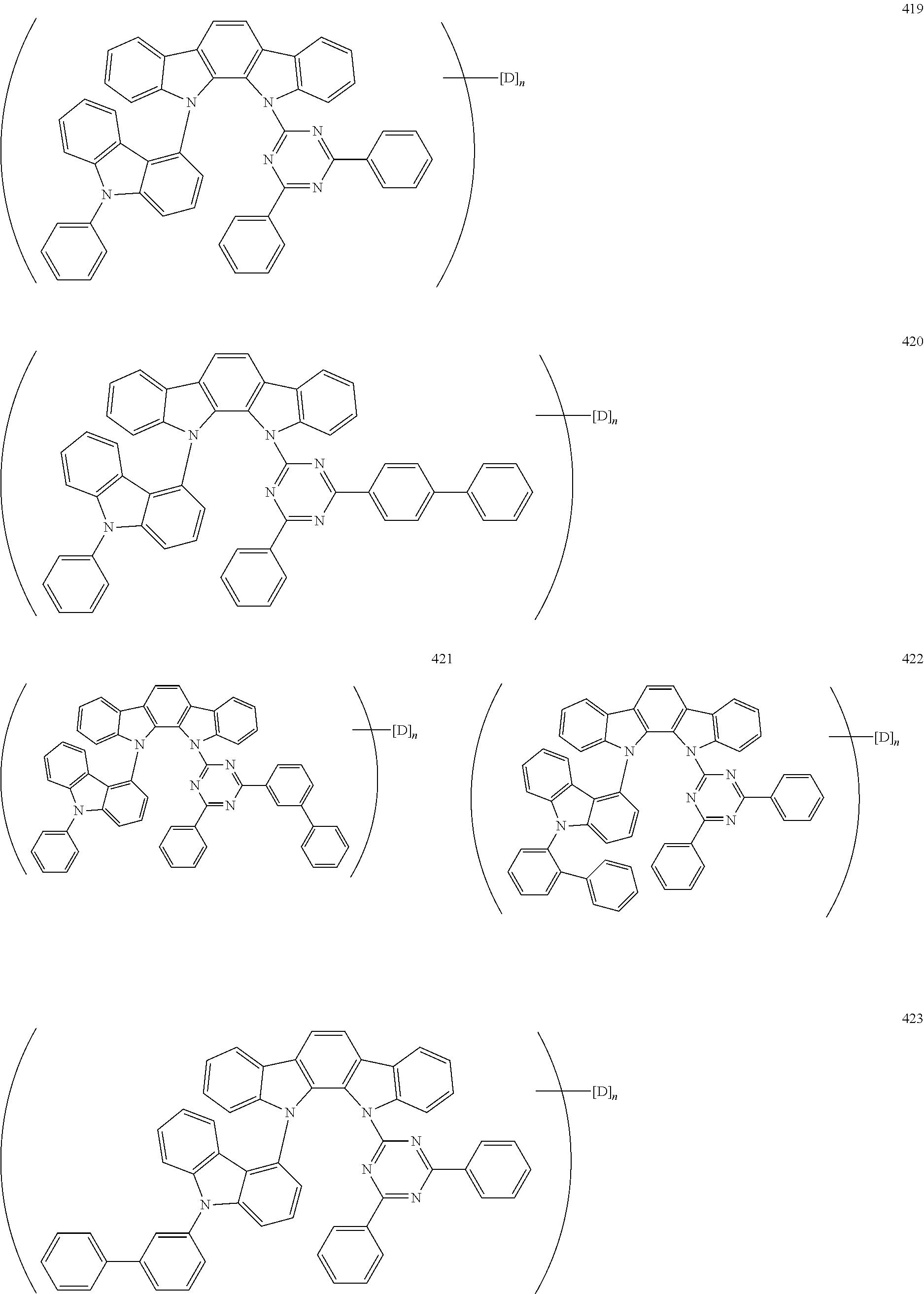

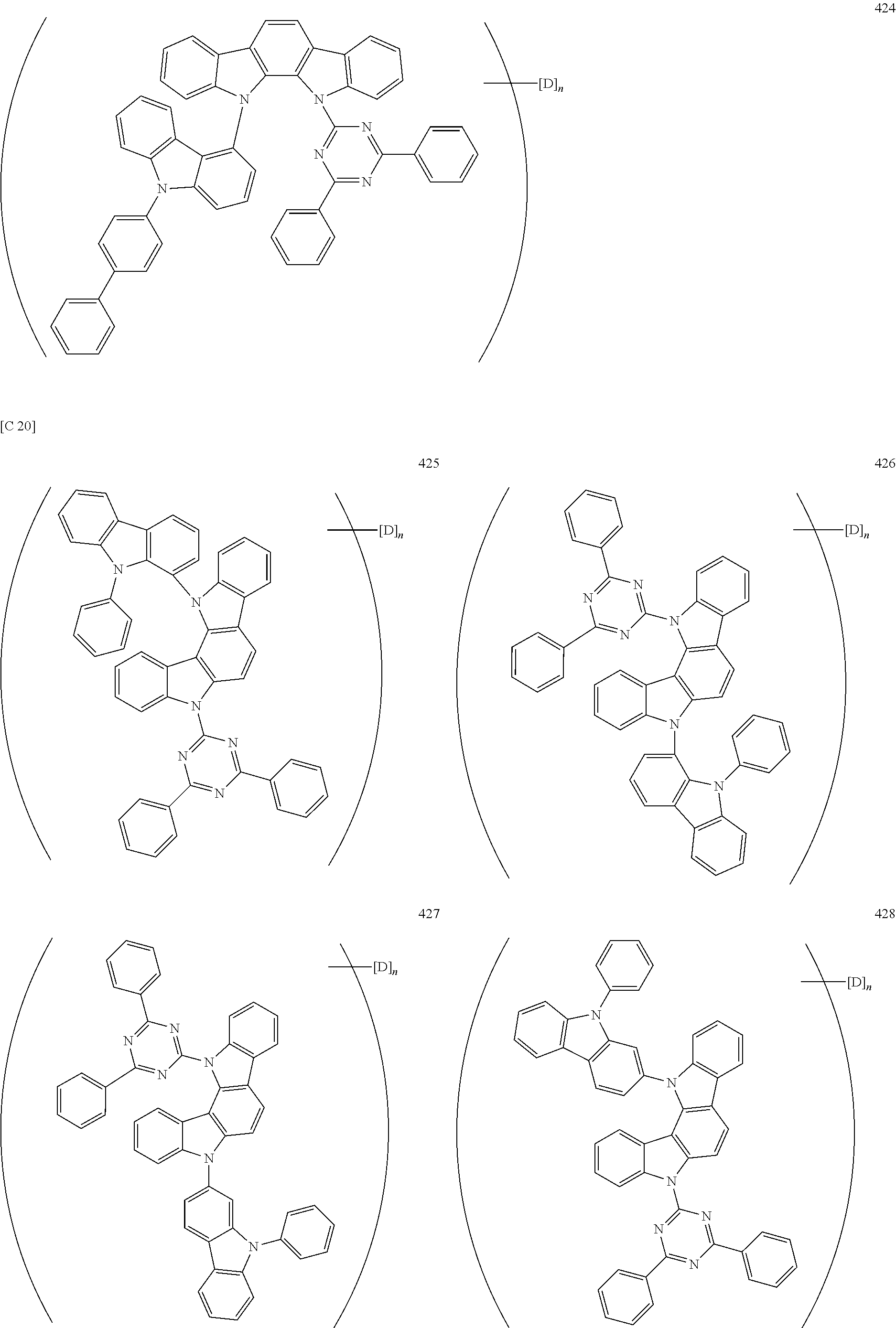

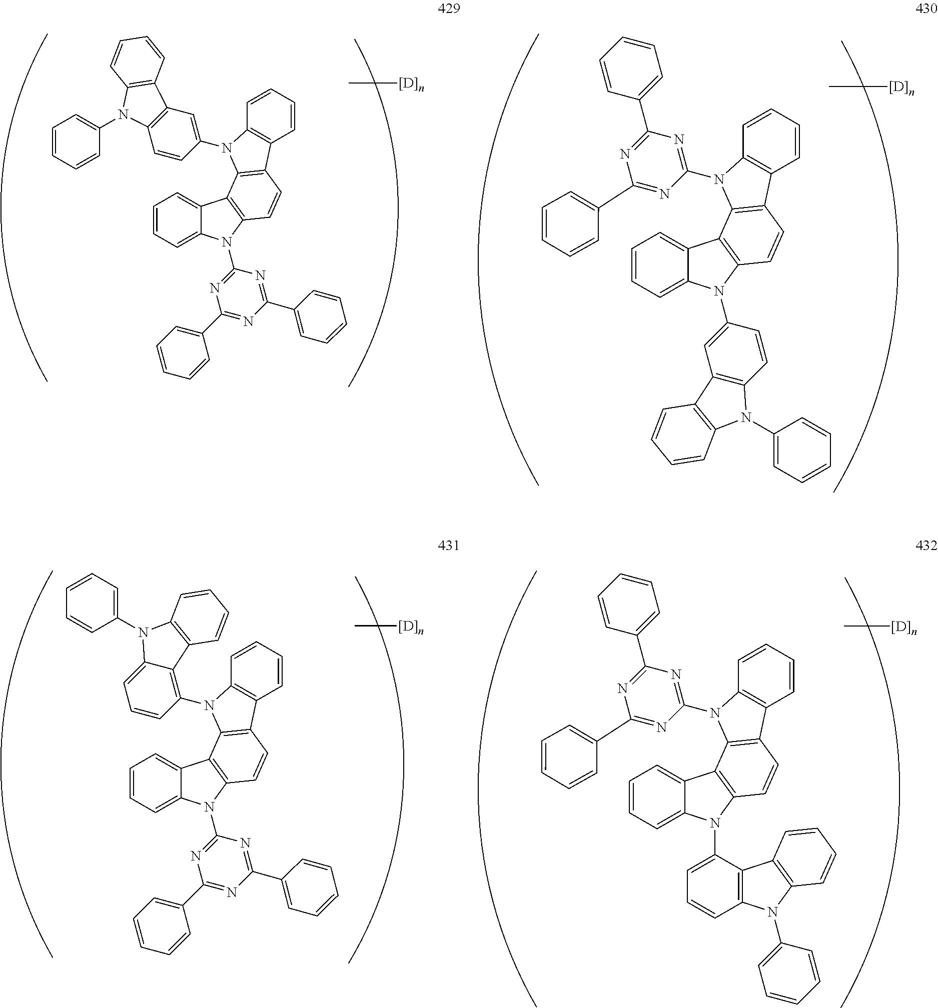

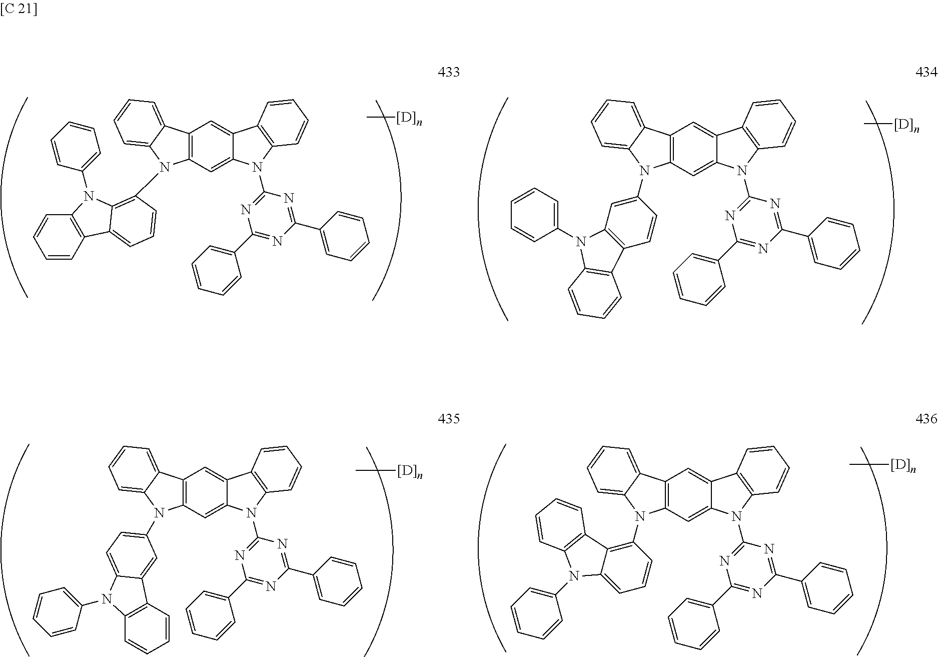

Among these exemplified compounds specifically shown, examples preferably include compounds 101, 102, 103, 109, 110, 111, 124, 127, 130, 136, 137, 138, 144, 145, 146, 159, 162, 165, 171, 172, 173, 179, 180, 181, 194, 197, 200, 206, 207, 208, 214, 215, 216, 229, 232, 235, 401, 402, 403, 404, 405, 406, 40?, 408, 409, 410, 411, 412, 413, 414, 415, 416, 417, 418, 419, 420, 421, 422, 423 and 424.

In the general formula (2), Ar⁴ and Ar⁵ each independently represent a substituted or unsubstituted aromatic hydrocarbon group having 6 to 14 carbon atoms, or a substituted or unsubstituted linked aromatic group in which two of these aromatic hydrocarbon groups are linked to each other. Ar⁴ and Ar⁵ each preferably represent a substituted or unsubstituted aromatic hydrocarbon group having 6 to 10 carbon atoms, or a substituted or unsubstituted linked aromatic group in which two of these aromatic hydrocarbon groups are linked to each other, more preferably represent a substituted or unsubstituted phenyl group, a substituted or unsubstituted biphenyl group, a substituted or unsubstituted naphthyl group, or a substituted or unsubstituted phenanthryl group, more preferably represent an unsubstituted phenyl group or biphenyl group. The biphenyl group has a structure in which two benzene rings are linked, and the binding position of each of the benzene rings may be any of the o-, m-, and p-positions. The linked aromatic group may be a group in which the linked aromatic hydrocarbon groups are the same as each other or such a group in which these groups are different from each other.

Specific examples of the unsubstituted aromatic hydrocarbon group having 6 to 14 carbon atoms, or the unsubstituted linked aromatic group in which two of these aromatic groups are linked to each other, in Ar⁴ and Ar⁵, include a group generated from benzene, naphthalene, phenanthrene, or compounds in which two of these are linked to each other.

L¹ and L² represent a direct bond or a substituted or unsubstituted phenylene group. The phenylene group may be bound at any of the ortho-, meta- and para-positions.

y and z represent the number of substitutions, independently represent 1 or 2, and preferably represent 1. When 2 is represented, L¹ or L² represents a trivalent phenylene group.

The general formula (2) is preferably formula (12). In the formula (12), the same symbol as that in the general formula (2) has the same meaning as that in the general formula (2).

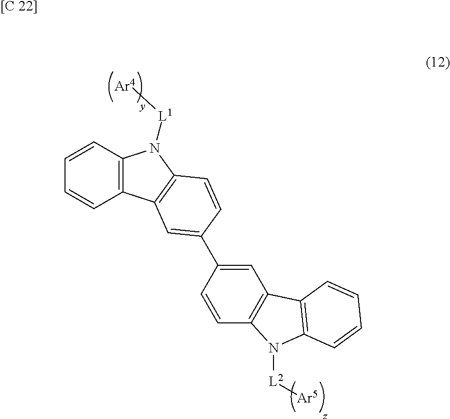

Specific examples of the general formula (2) are shown below, but are not limited to these exemplified compounds. In the following structural formula, D represents deuterium, and the meaning of m is the same as in n described above.

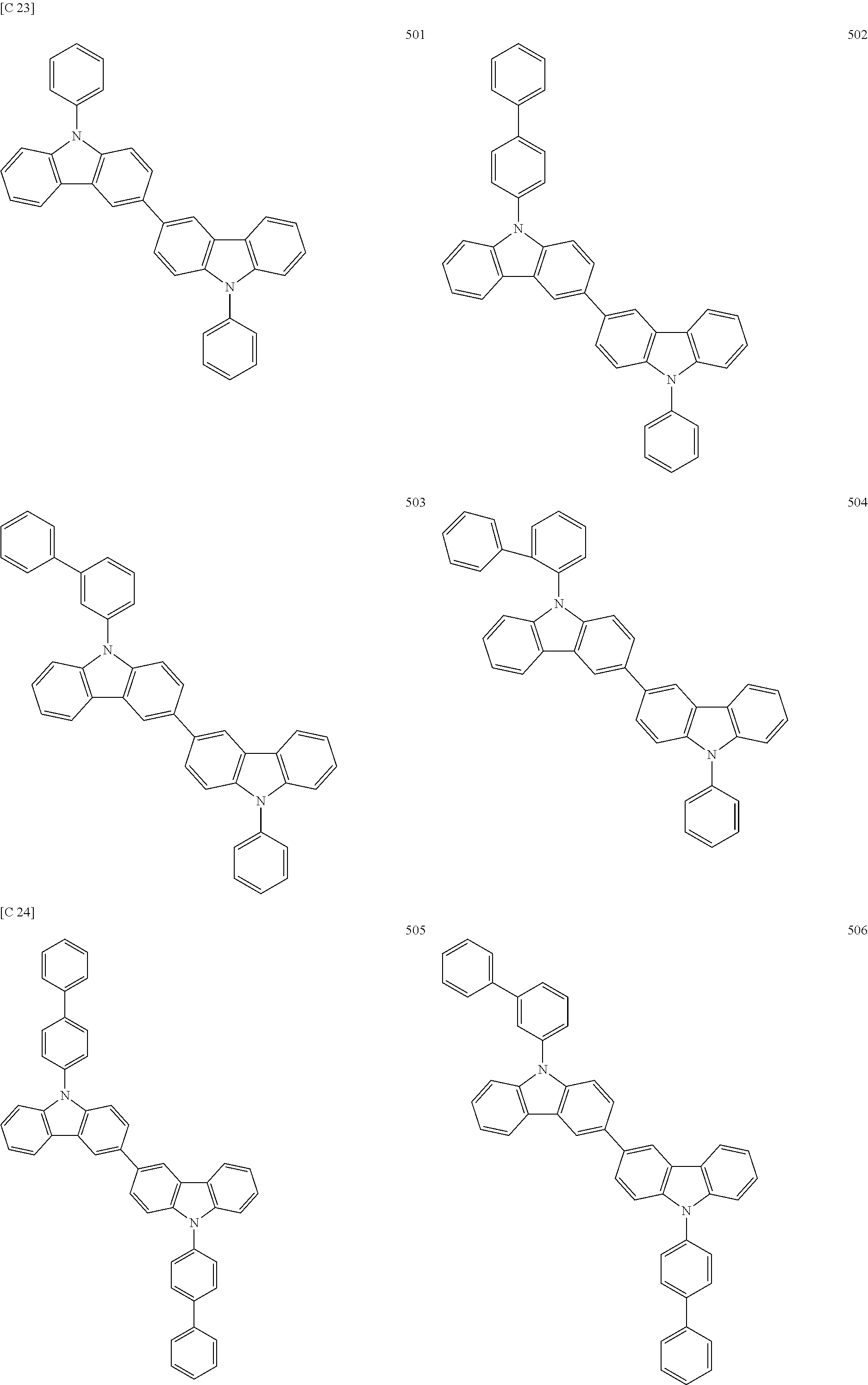

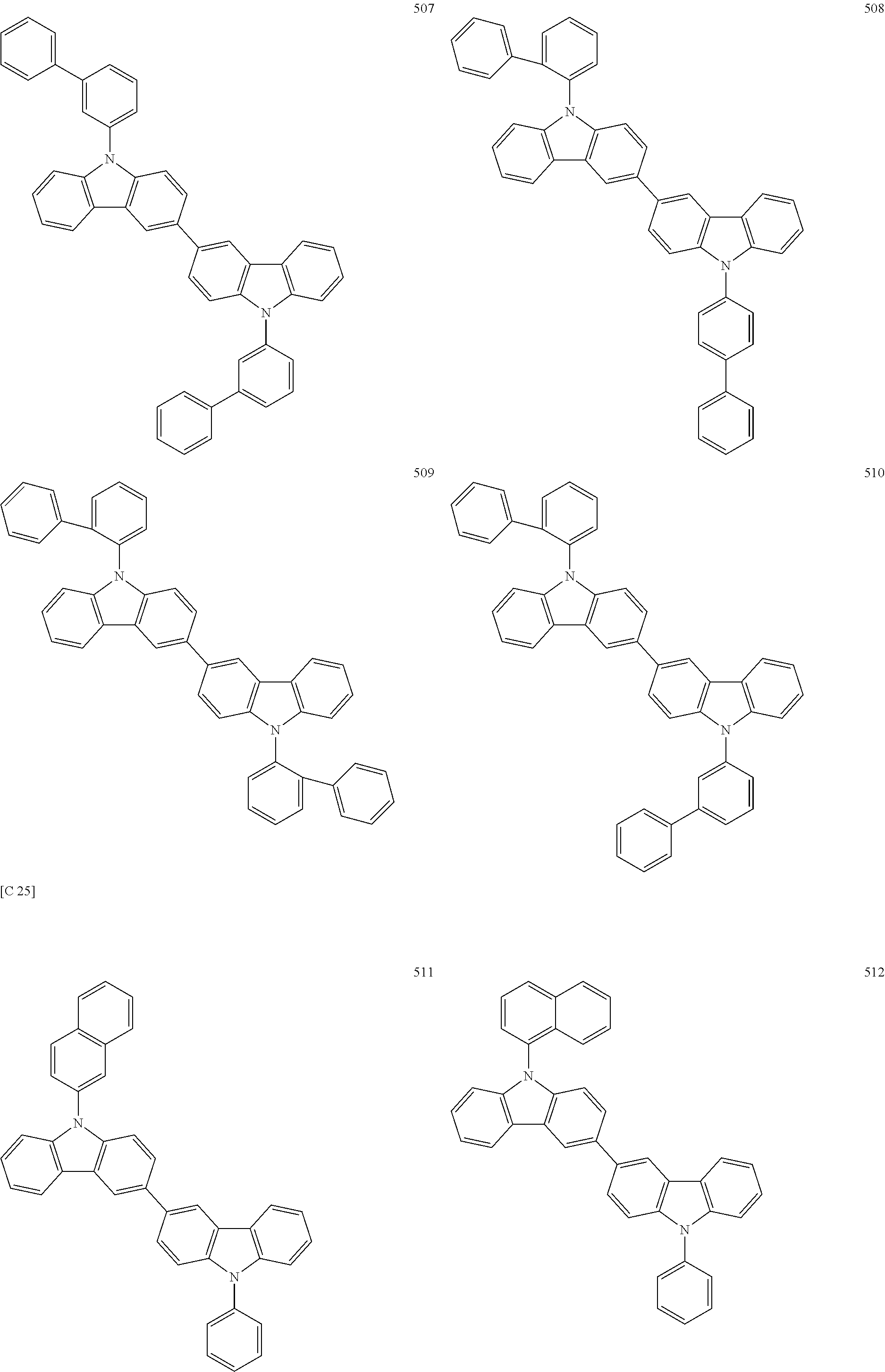

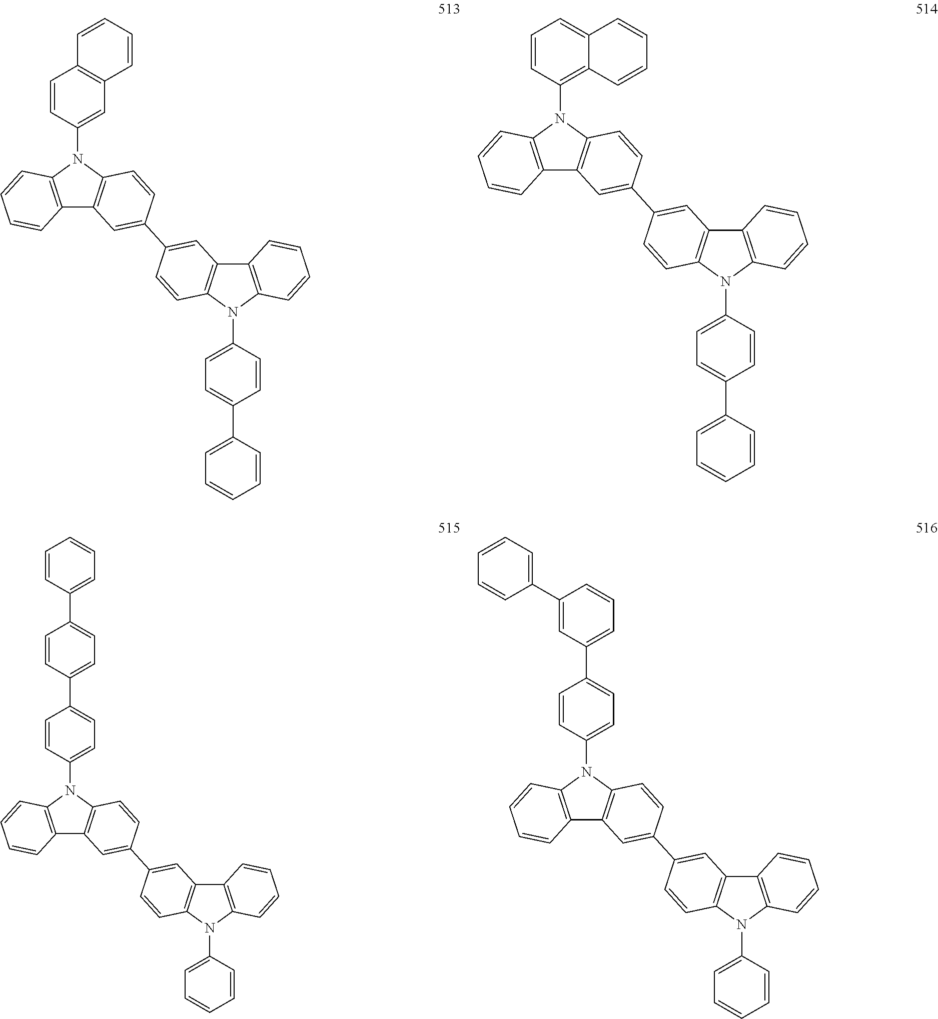

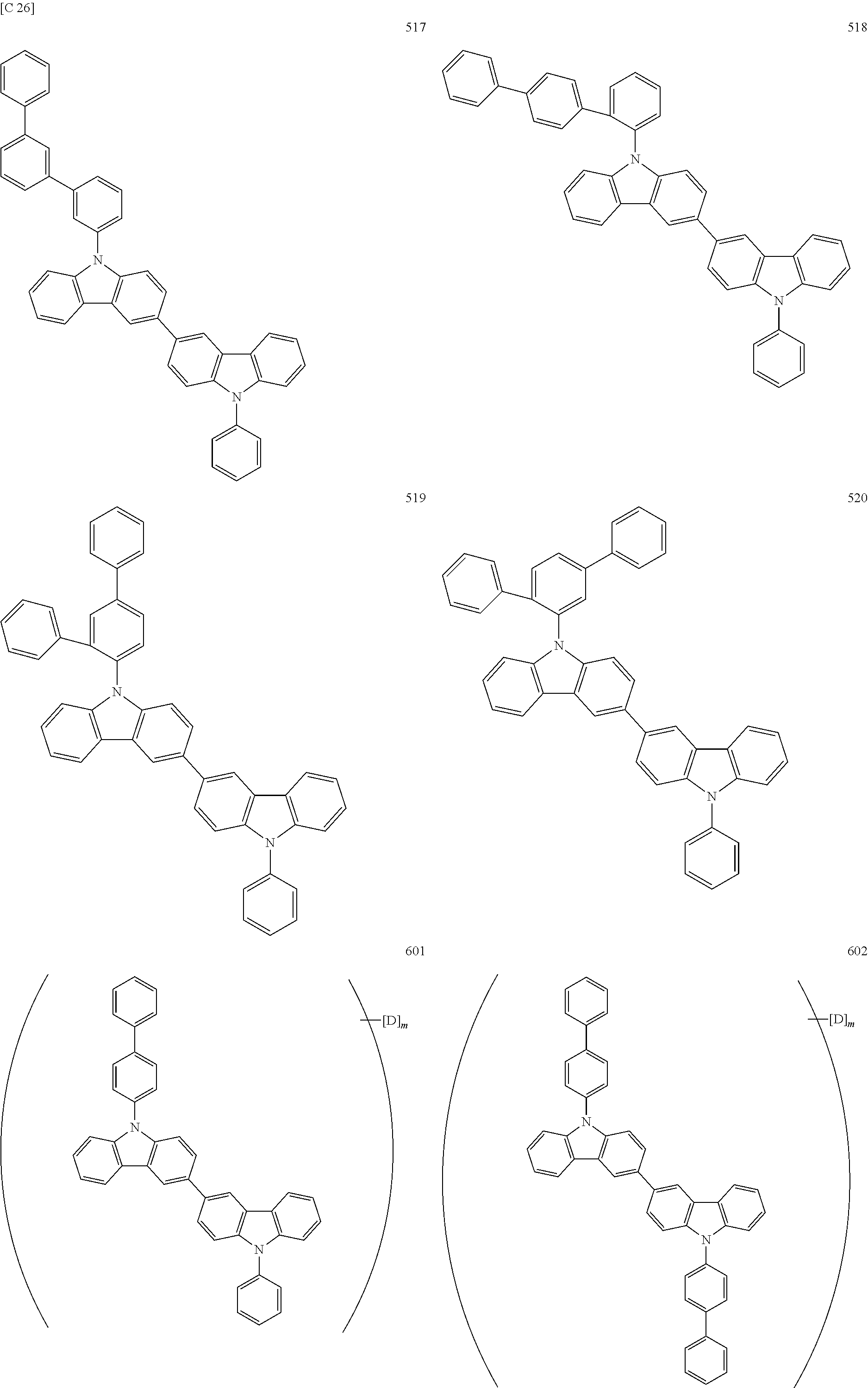

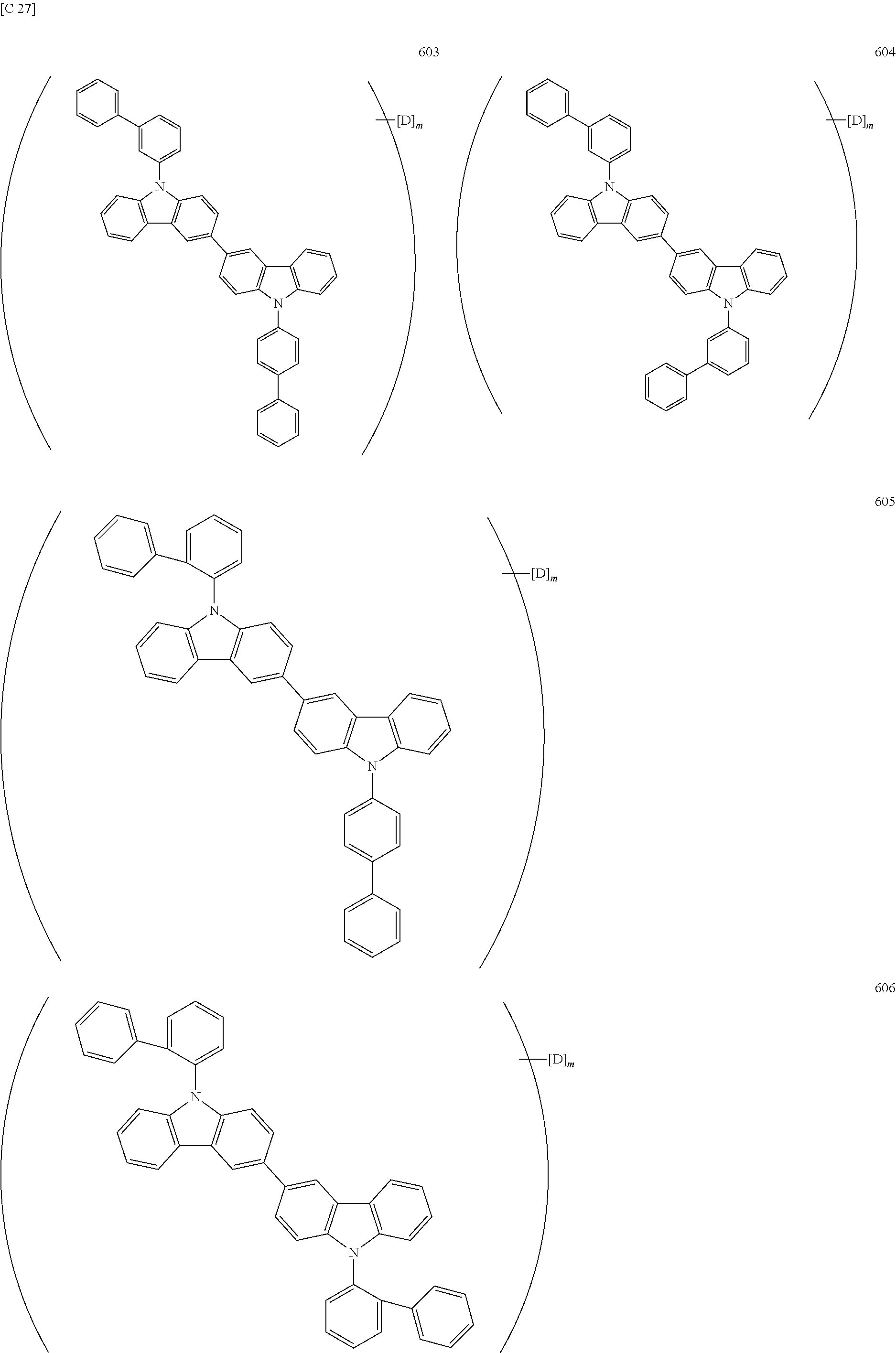

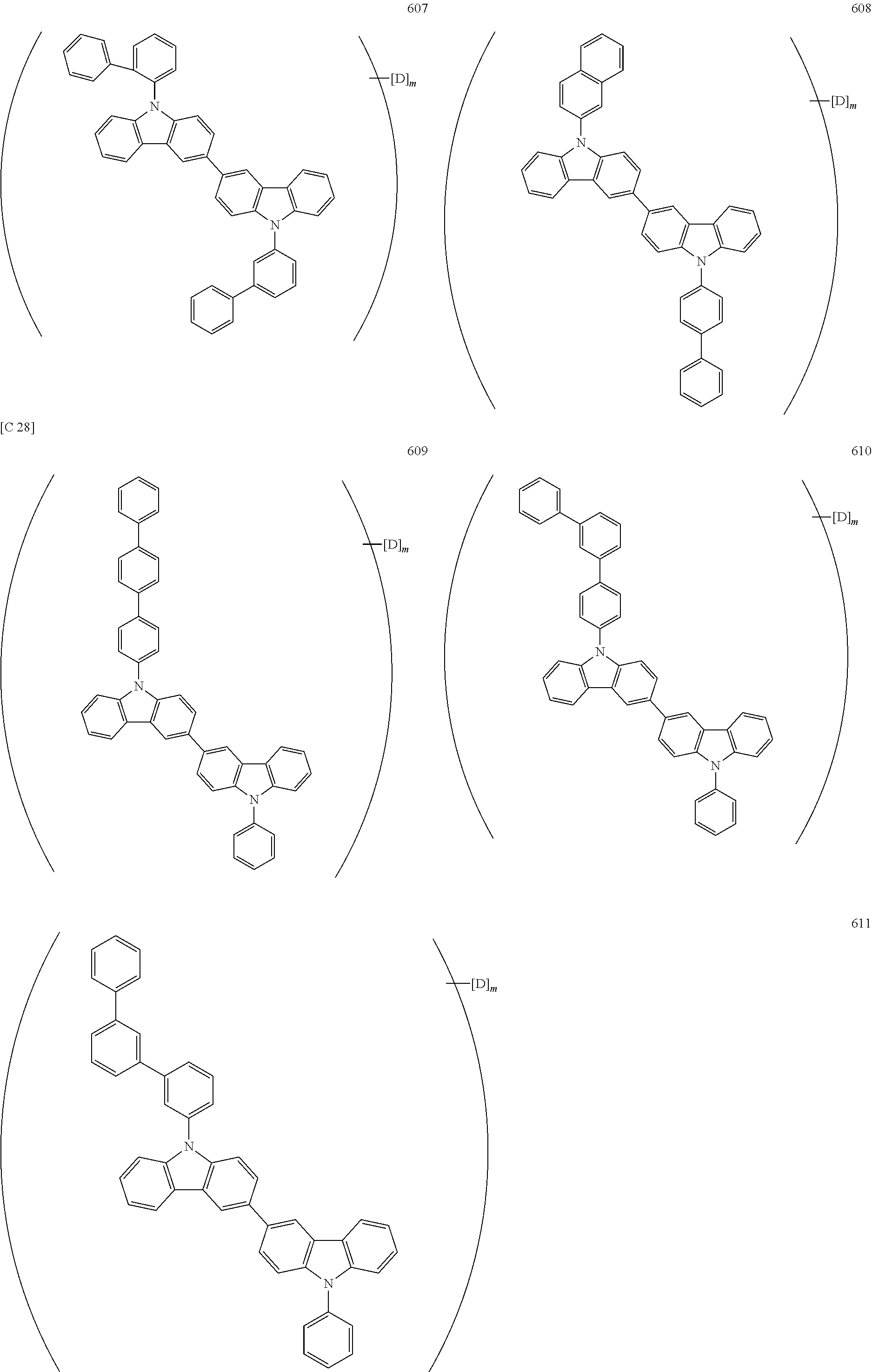

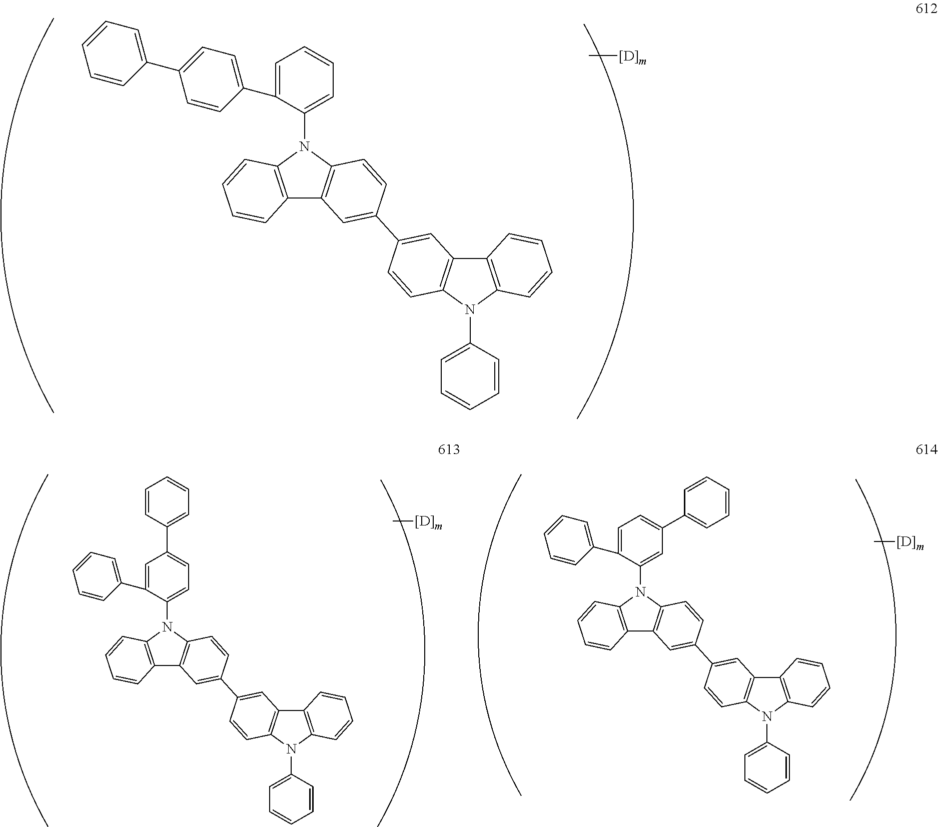

Among these exemplified compounds specifically shown, examples preferably include compounds 502, 503, 505, 506, 507, 508, 509, 510, 511, 513, 515, 516, 517, 518, 519, 520, 601, 602, 603, 604, 605, 606, 607, 608, 609, 610, 611, 612, 613, and 614.

Hydrogen in the compounds included in the mixed composition of the present invention may also be deuterium. In other words, in the general formula (1), hydrogen in a fused ring (indolocarbazole ring) containing the ring A, hydrogen in the group with which the indolocarbazole ring is substituted or in the carbazolyl group, and hydrogen in the aromatic rings in Ar¹, Ar², Ar³, and R¹, and furthermore hydrogen in the substituents with which these aromatic rings are substituted may be partially or fully deuterium. Hydrogen in two carbazole rings in the compound represented by the general formula (2), hydrogen in the aromatic rings in Ar⁴, Ar⁵, L¹ and L², and hydrogen in the substituents in Ar⁴, Ar⁵, L¹ and L² may be partially or fully deuterium.

In the case of a deuteride in which hydrogen in these compounds is partially or fully deuterated, the compound represented by the general formula (1) or the general formula (2) encompasses both a case of the compound that is a single compound and a case of a mixture of two or more such compounds. In other words, the compound represented by the general formula (1) or the general formula (2) may be in the form of two or more compounds encompassed in these formulas, or a mixture of such compounds different in number of deuterations and/or deuteration position.

All the compounds included in the mixed composition may be deuterated, or only some of these compounds may be deuterated.

The average rate of deuteration in the compound represented by the general formula (1) or the general formula (2) is each independently preferably 30% or more, more preferably 40% or more, further preferably 50, or more.

The average rate of deuteration is here specifically described, and a case of an average rate of deuteration of 50% means that half, on average, of all hydrogen is replaced by deuterium.

The average rate of deuteration can be determined by mass analysis or a proton nuclear magnetic resonance method. For example, when the rate is determined by a proton nuclear magnetic resonance method, a measurement sample is first prepared by adding and dissolving a compound and an internal standard material to and in a deuterated solvent, and the proton concentration [mol/g] in the compound included in the measurement sample is calculated from the ratio between integrated intensities derived from the internal standard material and the compound. Next, the ratio between the proton concentration in a deuterated compound and the proton concentration in the corresponding non-deuterated compound is calculated and subtracted from 1, and thus the average rate of deuteration in the deuterated compound can be calculated.

The mixed composition of the present invention comprises the compound represented by the general formula (1) and the compound represented by the general formula (2), and the mixing ratio (weight ratio) of the compound represented by the general formula (1) based on the total of both the compounds is preferably 20 to 70 wt %, more preferably 30 to 60 wt %, further preferably 40 to 50 wt %.

The mixed composition can comprise, in addition to the compound represented by the general formula (1) and the compound represented by the general formula (2), any other compound. Such other compound is, for example, a known host material or light-emitting dopant. However, the compound represented by the general formula (1) and the compound represented by the general formula (2) preferably occupy 50 wt % or more, more preferably 75 wt % or more of the whole composition.

The mixed composition of the present invention is suitable as a material or component of an organic EL device.

The mixed composition, when serves as a component of an organic EL device, is included in an organic layer of the organic EL device, and the organic layer is selected from the group consisting of a light-emitting layer, a hole injection layer, a hole transport layer, an electron transport layer, an electron injection layer, a hole blocking layer and an electron blocking layer. Preferred is a light-emitting layer, and the light-emitting layer preferably contains at least one light-emitting dopant.

The mixed composition of the present invention, when included in the light-emitting layer, is desirably included as a host of the light-emitting layer. Advantageously, it is preferable that the compound represented by the general formula (1) be included as a first host and the compound represented by the general formula (2) be included as a second host.

When the mixed composition of the present invention serves as a component of an organic EL device, a method can also be adopted which includes vapor-depositing a plurality compounds, for example, the compound represented by the general formula (1) and the compound represented by the general formula (2), by evaporation from each individual different vapor deposition source, or previously mixing these compounds before vapor deposition to provide a premixture, and then evaporating and vapor-depositing the premixture simultaneously from one vapor deposition source, to form an organic layer, preferably a light-emitting layer.

When the premixture is used as the mixed composition of the present invention to form a light-emitting layer, a necessary light-emitting dopant material, or another host to be used as necessary may be mixed, but when there is a large difference in temperatures to provide desired vapor pressure, such a light-emitting dopant material or another host may be vapor-deposited from another vapor deposition source.

When the mixed composition of the present invention is the premixture, 50% weight reduction temperatures of the compound represented by the general formula (1) and the compound represented by the general formula (2) are desirably within 20° C., more preferably within 15° C., in order to stably perform vapor deposition.

The organic EL device of the present invention has a plurality of organic layers between electrodes opposite to each other, and at least one of the organic layers is a light-emitting layer. At least one light-emitting layer preferably contains the mixed composition as a host. When the light-emitting layer contains the mixed composition, at least one light-emitting dopant is preferably contained.

Next, the structure of the organic EL device of the present invention will be described by referring to the drawing, but the structure of the organic EL device of the present invention is not limited thereto.

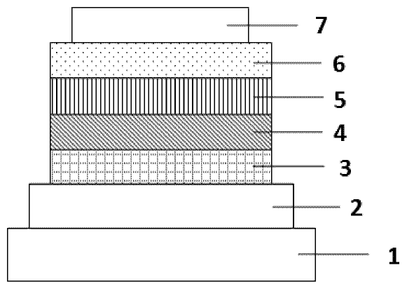

FIG. 1 is a cross-sectional view showing a structure example of an organic EL device generally used for the present invention, in which there are indicated a substrate 1, an anode 2, a hole injection layer 3, a hole transport layer 4, a light-emitting layer 5, an electron transport layer 6, and a cathode 7. The organic EL device of the present invention may have an exciton blocking layer adjacent to the light-emitting layer and may have an electron blocking layer between the light-emitting layer and the hole injection layer. The exciton blocking layer can be inserted into either of the anode side and the cathode side of the light-emitting layer and inserted into both sides at the same time. The organic EL device of the present invention has the anode, the light-emitting layer, and the cathode as essential layers, and preferably has a hole injection transport layer and an electron injection transport layer in addition to the essential layers, and further preferably has a hole blocking layer between the light-emitting layer and the electron injection transport layer. Note that the hole injection transport layer refers to either or both of a hole injection layer and a hole transport layer, and the electron injection transport layer refers to either or both of an electron injection layer and an electron transport layer.

A structure reverse to that of FIG. 1 is applicable, in which a cathode 7, an electron transport layer 6, a light-emitting layer 5, a hole transport layer 4, a hole injection layer 3, and an anode 2 are laminated on a substrate 1 in this order. In this case, layers may be added or omitted as necessary.

### Substrate

The organic EL device of the present invention is preferably supported on a substrate. The substrate is not particularly limited, and those conventionally used in organic EL devices may be used, and substrates made of, for example, glass, a transparent plastic, or quartz may be used.

### Anode

Regarding an anode material for an organic EL device, it is preferable to use a material of a metal, an alloy, an electrically conductive compound, and a mixture thereof, each having a large work function (4 eV or more). Specific examples of such an electrode material include a metal such as Au, and a conductive transparent material such as CuI, indium tin oxide (ITO), SnO₂, and ZnO. In addition, an amorphous material such as IDIXO (In2O3-ZnO), which is capable of forming a transparent conductive film, may be used. Regarding the anode, such an electrode material is used to form a thin film by, for example, a vapor-deposition or sputtering method, and a desired shape pattern may be formed by a photolithographic method; or if the pattern accuracy is not particularly required (about 100 μm or more), a pattern may be formed via a desired shape mask when the electrode material is vapor-deposited or sputtered. Alternatively, when a coatable substance such as an organic conductive compound is used, a wet film formation method such as a printing method or a coating method may be used. For taking emitted light from the anode, it is desired to have a transmittance of more than 10%, and the sheet resistance for the anode is preferably several hundreds Ω/or less. The film thickness is selected usually within 10 to 1000 nm, preferably within 10 to 200 nm though depending on the material.

### Cathode

Regarding a cathode material, preferable to a material of a metal (an electron injection metal), an alloy, an electrically conductive compound, or a mixture thereof, each having a small work function (4 eV or less) are used. Specific examples of such an electrode material include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and a rare earth metal. Among these, from the viewpoint of the electron injectability and the durability against oxidation and the like, a mixture of an electron injection metal and a second metal which is a stable metal having a larger work function value is suitable, and examples thereof include a magnesium/silver mixture, a magnesium/aluminum mixture, a magnesium/indium mixture, an aluminum/aluminum oxide mixture, a lithium/aluminum mixture and aluminum. The cathode can be produced by forming a thin film by a method such as vapor-depositing or sputtering of such a cathode material. In addition, the sheet resistance of cathode is preferably several hundreds Ω/or less. The film thickness is selected usually within 10 nm to 5 μm, preferably within 50 to 200 nm. Note that for transmission of emitted light, if either one of the anode and cathode of the organic EL device is transparent or translucent, emission luminance is improved, which is convenient.

In addition, formation of a film of the above metal with a thickness of 1 to 20 nm on a cathode, followed by formation of a conductive transparent material described in the description on the anode thereon, enables production of a transparent or translucent cathode, and application of this enables production of a device wherein an anode and a cathode both have light transmittance.

### Light-Emitting Layer

The light-emitting layer is a layer that emits light after excitons are generated when holes and electrons injected from the anode and the cathode, respectively, are recombined. As a light-emitting layer, a light-emitting dopant material and a host are contained.

The mixed composition of the present invention can be suitably used as a material for an organic electroluminescent device, and can be preferably used as a host. The host is preferably one in which the compound represented by the general formula (1) is used as a first host and the compound represented by the general formula (2) is used as a second host. Regarding the first host or the second host, one kind of compound may be used, or two or more different compounds may be used. If necessary, one or more other known host materials may be used in combination; however, it is preferable that an amount thereof to be used be 50 wt % or less, preferably 25 wt % or less based on the host materials in total.

The method for producing an organic electroluminescent device of the present invention comprises a step of providing the above premixture, and vapor-depositing the premixture from one vapor deposition source, to form a light-emitting layer. A more preferred method comprises vapor-depositing the premixture by vaporization from a single vapor deposition source. Herein, the premixture is suitably a uniform composition.

When the first host and the second host are premixed and used, it is desirable that a difference in 50% weight reduction temperature (T50) be small in order to produce an organic EL device having favorable characteristics with high reproducibility. The 50% weight reduction temperature is a temperature at which the weight is reduced by 50% when the temperature is raised to 550° C. from room temperature at a rate of 10° C./min in TG-DTA measurement under a nitrogen stream reduced pressure (1 Pa). It is considered that vaporization due to evaporation or sublimation the most vigorously occurs around this temperature.

The difference in 50% weight reduction temperatures is preferably within 20° C. because the premixture can be vaporized from a single vapor deposition source and vapor-deposited to thereby obtain a uniform vapor-deposited film. In this case, the premixture may be mixed with a light-emitting dopant material necessary for formation of a light-emitting layer, or another host to be used as necessary.

The method for providing the premixture by premixing is desirably a method that can allow for mixing of the first host and the second host as uniformly as possible, and examples thereof include pulverization and mixing, and sublimination. Examples also include a method including heating and melting a mixture containing the first host and the second host to a temperature equal to or more than the melting point(s) of both or one of the first host and the second host under reduced pressure, for example, under a reduced pressure of 200 Pa or less, or under an atmosphere of an inert gas such as nitrogen, but not limited thereto. In the case of melting, the premixture may be in a molten state, or is suitably obtained by cooling and solidifying such a molten mixture.

The premixture may be in powder, stick, or granule form.

In a case where the compound represented by the general formula (1) and the compound represented by the general formula (2) are each a deuteride, a production method in which a starting material fully or partially deuterated is used and a production method according to hydrogen/deuterium exchange reaction are known. Such a raw material fully or partially deuterated can be purchased from a supplier of a commercially available product, or can be produced according to known hydrogen/deuterium exchange reaction. Examples of such known hydrogen/deuterium exchange reaction include a method including allowing a non-deuterated substance to act on deuterium gas or an equivalent thereof in the presence of a transition metal catalyst, and a method including treating a non-deuterated substance with a deuterated solvent (deuterated benzene or the like) in the presence of an acid catalyst.

When a phosphorescent dopant is used as a light-emitting dopant material, preferred is a phosphorescent dopant including an organic metal complex containing at least one metal selected from ruthenium, rhodium, palladium, silver, rhenium, osmium, iridium, platinum and gold. Specifically, iridium complexes described in J. Am. Chem. Soc. 2001, 123, 4304, JP2013-530515A, US2016/0049599A, US2017/0069848A, US2018/0282356A, US2019/0036043A, or the like, or platinum complexes described in US2018/0013078A, KR2018-094482A, or the like are preferably used, but the phosphorescent dopant is not limited thereto.

Regarding the phosphorescent dopant material, only one kind thereof may be contained in the light-emitting layer, or two or more kinds thereof may be contained. A content of the phosphorescent dopant material is preferably 0.1 to 30 wt % and more preferably 1 to 20 wt % with respect to the host material.

The phosphorescent dopant material is not particularly limited, and specific examples thereof include the following compounds.

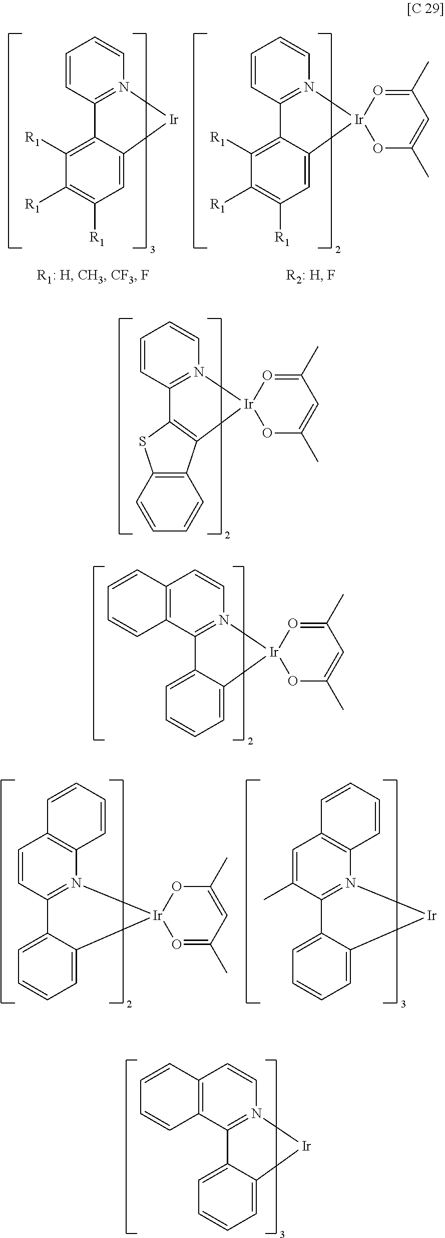

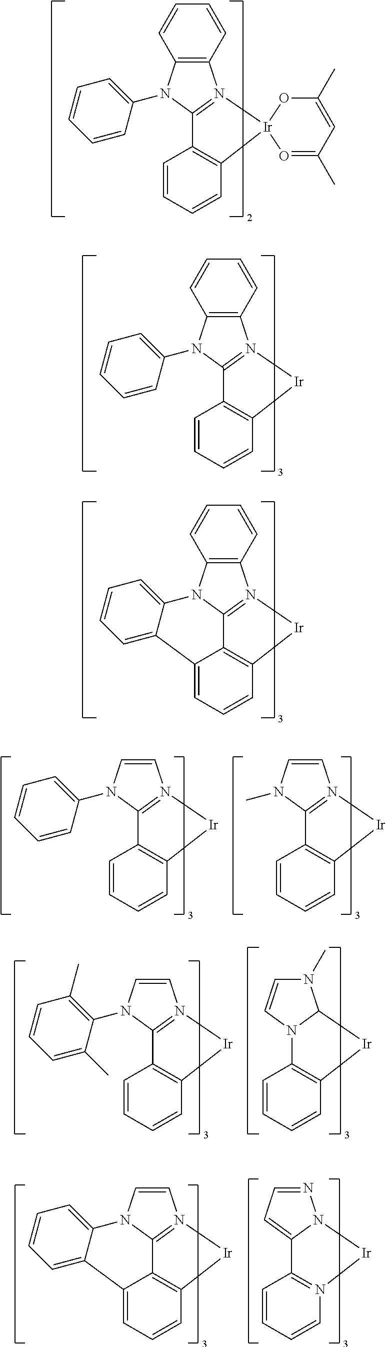

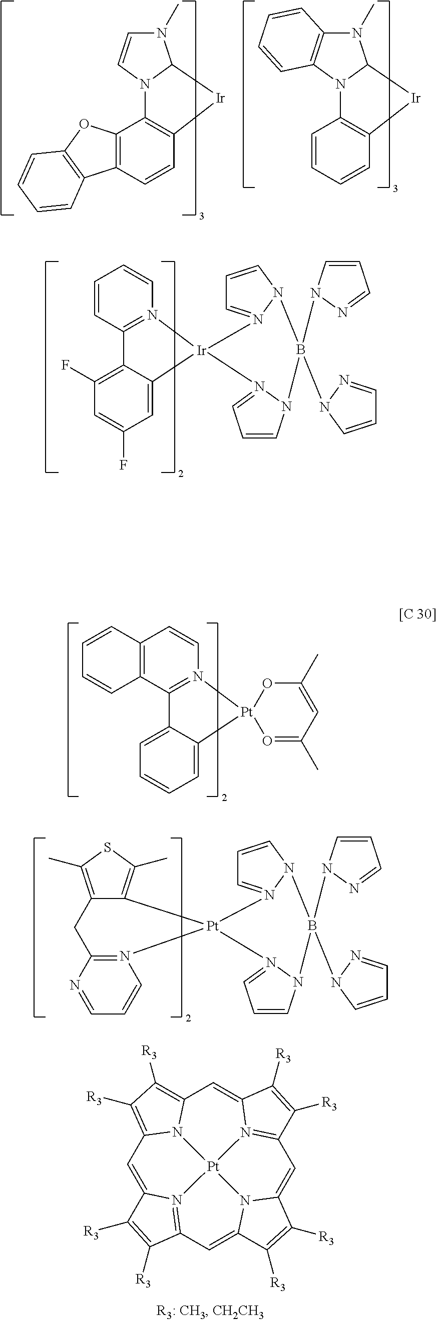

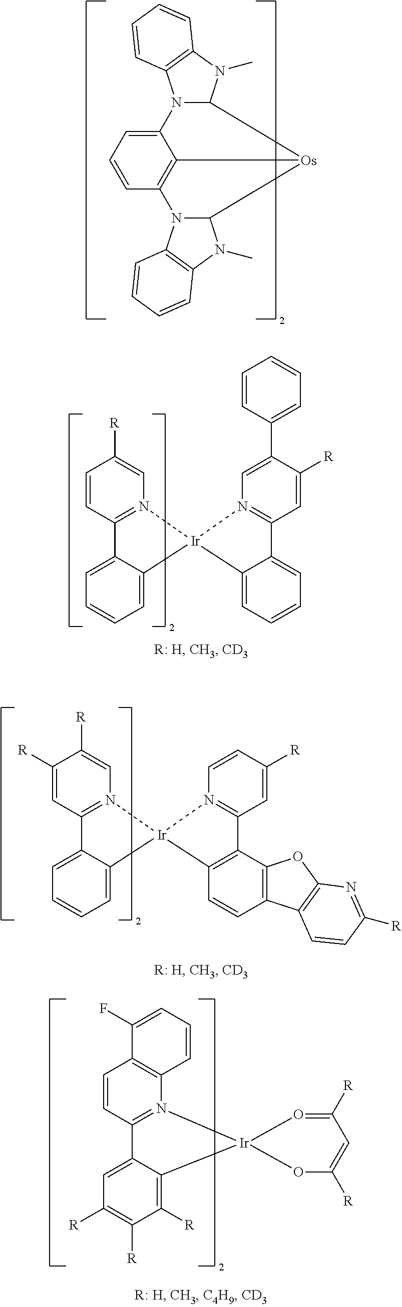

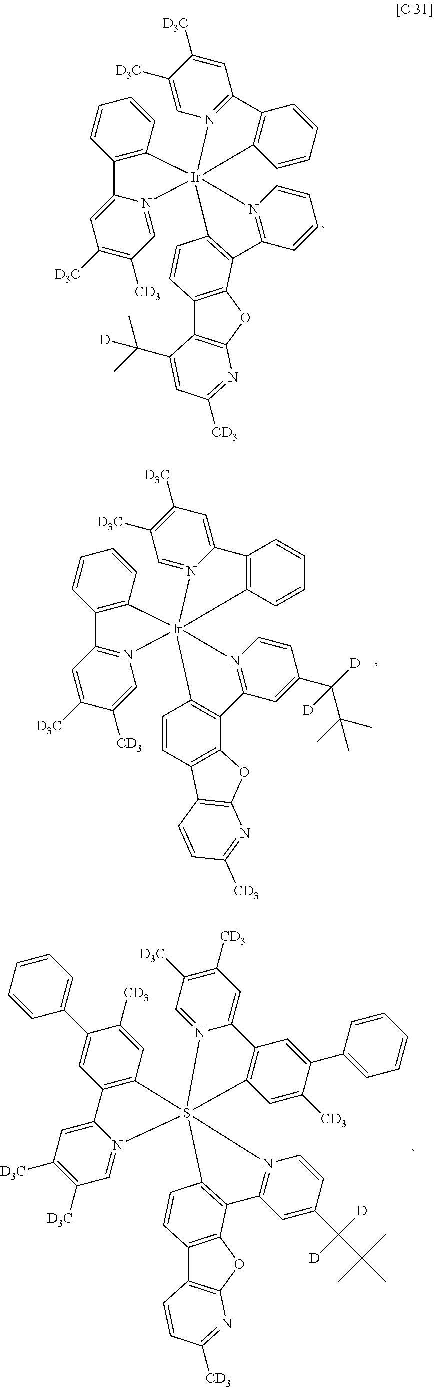

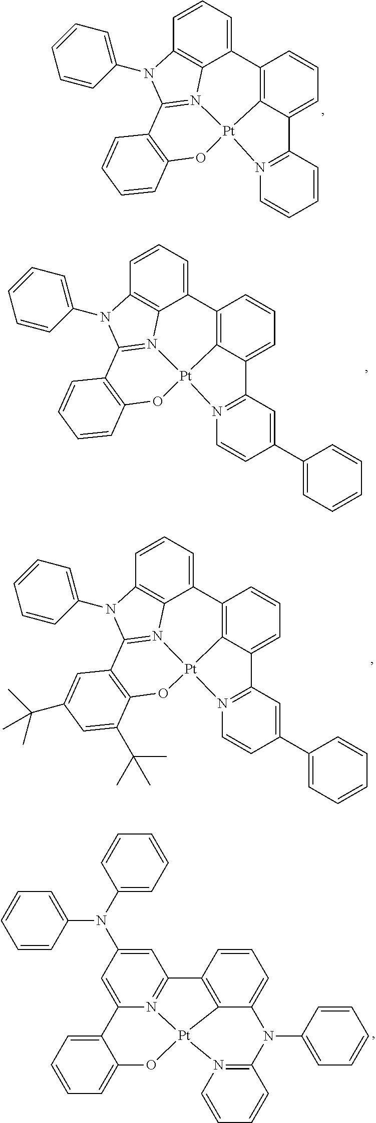

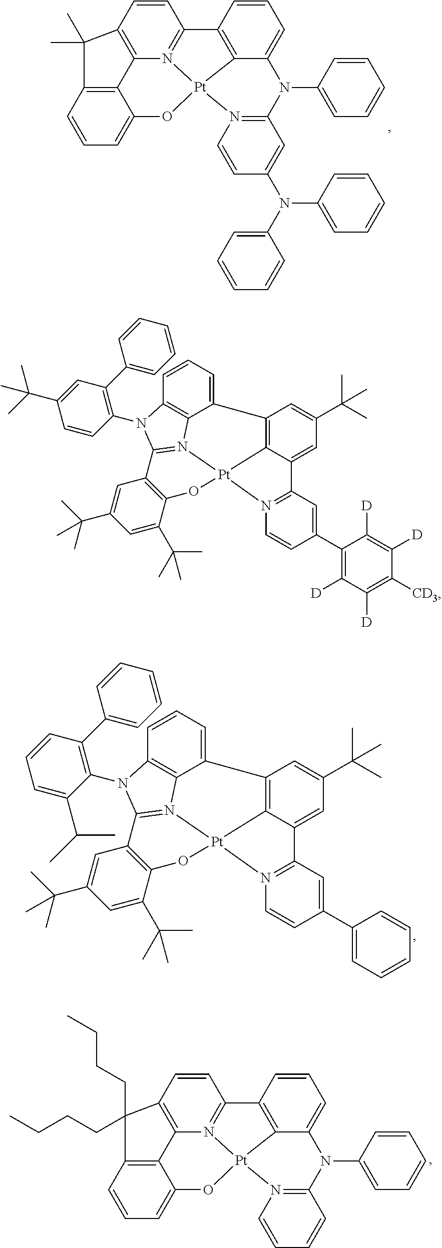

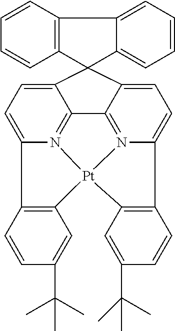

When a fluorescence-emitting dopant is used as the light-emitting dopant material, the fluorescence-emitting dopant is not particularly limited. Examples thereof include benzoxazole derivatives, benzothiazole derivatives, benzimidazole derivatives, styrylbenzene derivatives, polyphenyl derivatives, diphenylbutadiene derivatives, tetraphenyl butadiene derivatives, naphthalimido derivatives, coumarin derivatives, fused aromatic compounds, perinone derivatives, oxadiazole derivatives, oxazine derivatives, aldazine derivatives, pyrrolidine derivatives, cyclopentadiene derivatives, bisstyryl anthracene derivatives, quinacridone derivatives, pyrrolopyridine derivatives, thiadiazolopyridine derivatives, styrylamine derivatives, diketopyrrolopyrrole derivatives, aromatic dimethylidine compounds, metal complexes of 8-quinolinol derivatives or metal complexes of pyromethene derivatives, rare earth complexes, various metal complexes represented by transition metal complexes, polymer compounds such as polythiophene, polyphenylene, and polyphenylene vinylene, and organosilane derivatives. Preferred examples thereof include fused aromatic derivatives, styryl derivatives, diketopyrrolopyrrole derivatives, oxazine derivatives, pyromethene metal complexes, transition metal complexes, and lanthanoid complexes. More preferable examples thereof include naphthalene, pyrene, chrysene, triphenylene, benzo[c]phenanthrene, benzo[a]anthracene, pentacene, perylene, fluoranthene, acenaphthofluoranthene, dibenzo[a,j]anthracene, dibenzo[a,h]anthracene, benzo[a]naphthalene, hexacene, naphtho[2,1-f]isoquinoline, α-naphthaphenanthridine, phenanthrooxazole, quinolino[6,5-f]quinoline, and benzothiophanthrene. These may have an alkyl group, an aryl group, an aromatic heterocyclic group, or a diarylamino group as a substituent.

When a thermally activated delayed fluorescence-emitting dopant is used as the light-emitting dopant material, the thermally activated delayed fluorescence-emitting dopant is not particularly limited. Examples thereof include: metal complexes such as a tin complex and a copper complex; indolocarbazole derivatives described in WO2011/070963A; cyanobenzene derivatives and carbazole derivatives described in Nature 2012, 492, 234; and phenazine derivatives, oxadiazole derivatives, triazole derivatives, sulfone derivatives, phenoxazine derivatives, and acridine derivatives described in Nature Photonics 2014, 8,326.

The thermally activated delayed fluorescence-emitting dopant material is not particularly limited, and specific examples thereof include the following compound.

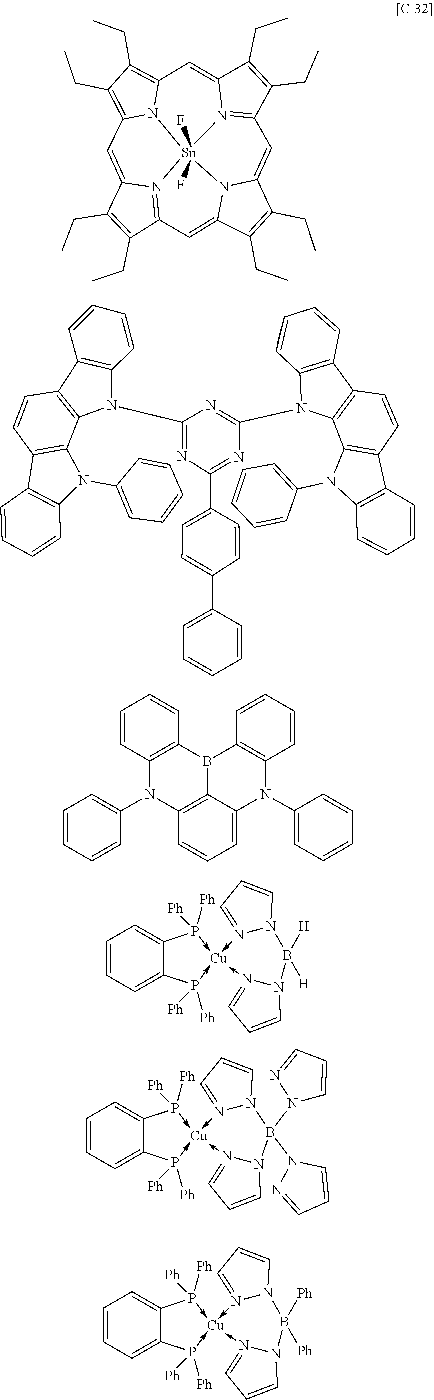

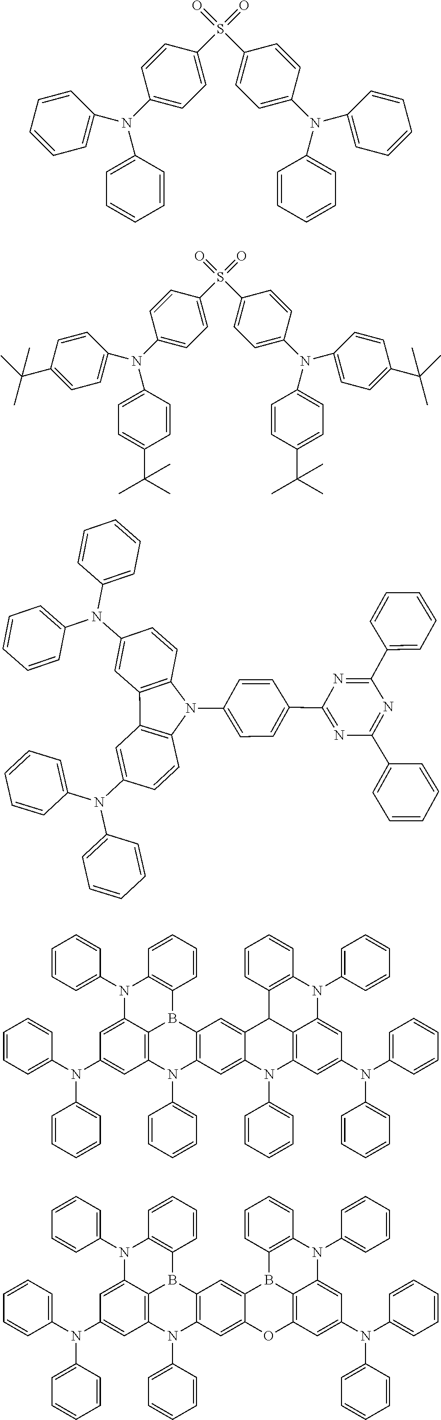

Regarding the thermally activated delayed fluorescence-emitting dopant material, only one kind thereof may be contained in the light-emitting layer, or two or more kinds thereof may be contained. In addition, the thermally activated delayed fluorescence-emitting dopant may be used by mixing with a phosphorescent dopant and a fluorescence-emitting dopant. A content of the thermally activated delayed fluorescence-emitting dopant material is preferably 0.1 wt % to 50 wt % and more preferably 1 wt % to 30 wt with respect to the host material.

### Injection Layer

The injection layer is a layer that is provided between an electrode and an organic layer in order to lower a driving voltage and improve emission luminance, and includes a hole injection layer and an electron injection layer, and may be present between the anode and the light-emitting layer or the hole transport layer, and between the cathode and the light-emitting layer or the electron transport layer. The injection layer can be provided as necessary.

### Hole Blocking Layer

The hole blocking layer has a function of the electron transport layer in a broad sense, and is made of a hole blocking material having a function of transporting electrons and a significantly low ability to transport holes, and can block holes while transporting electrons, thereby improving a probability of recombining electrons and holes in the light-emitting layer.

### Electron Blocking Layer

The electron blocking layer has a function of a hole transport layer in a broad sense and blocks electrons while transporting holes, thereby enabling a probability of recombining electrons and holes in the light-emitting layer to be improved.

Regarding the material of the electron blocking layer, a known electron blocking layer material can be used and a material of the hole transport layer to be described below can be used as necessary. A film thickness of the electron blocking layer is preferably 3 to 100 nm, and more preferably 5 to 30 nm.

### Exciton Blocking Layer

The exciton blocking layer is a layer for preventing excitons generated by recombination of holes and electrons in the light-emitting layer from being diffused in a charge transport layer, and insertion of this layer allows excitons to be efficiently confined in the light-emitting layer, enabling the luminous efficiency of the device to be improved. The exciton blocking layer can be inserted, in a device having two or more light-emitting layers adjacent to each other, between two adjacent light-emitting layers.

Regarding the material of the exciton blocking layer, a known exciton blocking layer material can be used. Examples thereof include 1,3-dicarbazolyl benzene (mCP) and bis(8-hydroxy-2-methylquinoline)-(4-phenylphenoxy)aluminum (III) (BAlq).

### Hole Transport Layer

The hole transport layer is made of a hole transport material having a function of transporting holes, and the hole transport layer can be provided as a single layer or a plurality of layers.

The hole transport material has either hole injection, transport properties or electron barrier properties, and may be an organic material or an inorganic material. For the hole transport layer, any one selected from conventionally known compounds can be used. Examples of such a hole transport material include porphyrin derivatives, arylamine derivatives, triazole derivatives, oxadiazole derivatives, imidazole derivatives, polyarylalkane derivatives, pyrazoline derivatives and pyrazolone derivatives, phenylenediamine derivatives, arylamine derivatives, amino-substituted chalcone derivatives, oxazole derivatives, styryl anthracene derivatives, fluorenone derivatives, hydrazone derivatives, stilbene derivatives, silazane derivatives, an aniline copolymer, and a conductive polymer oligomer, and particularly a thiophene oligomer. Use of porphyrin derivatives, arylamine derivatives, or styrylamine derivatives is preferred. Use of arylamine derivatives is more preferred.

### Electron Transport Layer

The electron transport layer is made of a material having a function of transporting electrons, and the electron transport layer can be provided as a single layer or a plurality of layers.

The electron transport material (which may also serve as a hole blocking material) may have a function of transferring electrons injected from the cathode to the light-emitting layer. For the electron transport layer, any one selected from conventionally known compounds can be used, and examples thereof include polycyclic aromatic derivatives such as naphthalene, anthracene, and phenanthroline, tris(8-hydroxyquinoline)aluminum(III) derivatives, phosphine oxide derivatives, nitro-substituted fluorene derivatives, diphenylquinone derivatives, thiopyrandioxide derivatives, carbodiimide, fluorenylidene methane derivatives, anthraquinodimethane and anthrone derivatives, bipyridine derivatives, quinoline derivatives, oxadiazole derivatives, benzimidazole derivatives, benzothiazole derivatives, and indolocarbazole derivatives. In addition, a polymer material in which the above material is introduced into a polymer chain or the above material is used for a main chain of a polymer can be used.

### EXAMPLES

Hereafter, the present invention will be described in detail by referring to Examples, but the present invention is not limited to these Examples and can be implemented in various forms without departing from the gist thereof.

### Synthesis Example 1

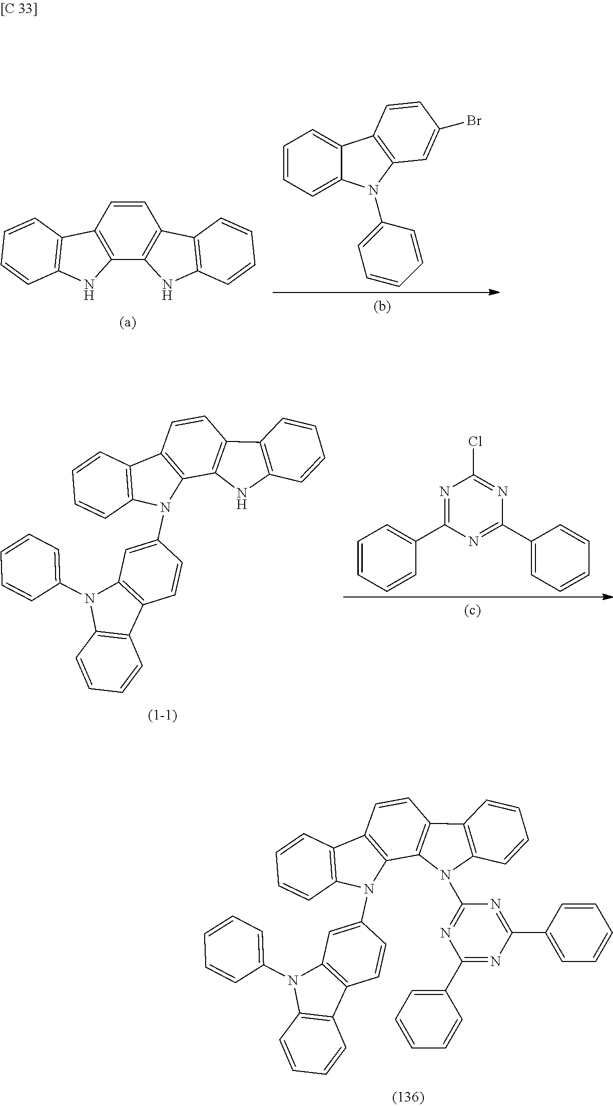 [SMILES: c1ccc2c(c1)[nH]c1c2ccc2c3ccccc3[nH]c21 ; Brc1ccc2c3ccccc3n(-c3ccccc3)c2c1 ; c1ccc(-n2c3ccccc3c3ccc(-n4c5ccccc5c5ccc6c7ccccc7[nH]c6c54)cc32)cc1 ; Clc1nc(-c2ccccc2)nc(-c2ccccc2)n1 ; c1ccc(-c2nc(-c3ccccc3)nc(-n3c4ccccc4c4ccc5c6ccccc6n(-c6ccc7c8ccccc8n(-c8ccccc8)c7c6)c5c43)n2)cc1]

To 61 g of compound (a) were added 94 g of compound (b), 4.5 g of copper iodide, 66 g of potassium carbonate, 0.18 g of 18-crown-6-ether, and 250 ml of 1,3-dimethyl-2-imidazolidinone, and stirred at 190° C. under a nitrogen atmosphere for 19 hours. After cooling to room temperature, the resultant was purified by silica gel chromatography and purified by crystallization to give 71 g (60% yield) of intermediate (1-1) as a white solid.

Under a nitrogen atmosphere, 4.2 g of 61 wt, sodium hydride was added to 60 ml of N,N′-dimethylacetamide (DMAc) to prepare a suspension. Thereto was added 40 g of intermediate (1-1) dissolved in 200 mL of DMAc, and stirred for 30 minutes. Thereto was added 26 g of compound (c), and then stirred for 3 hours. The reaction solution was added to a mixture solution of methanol (250 ml) and distilled water (120 ml) while being stirred, and the resulting precipitated solid was collected by filtration. The resulting solid was purified by silica gel chromatography and purified by crystallization to give 32 g (55% yield) of compound (136) as a yellow solid (APCI-TOFMS, m/z 729[M+H]⁺).

### Synthesis Example 2

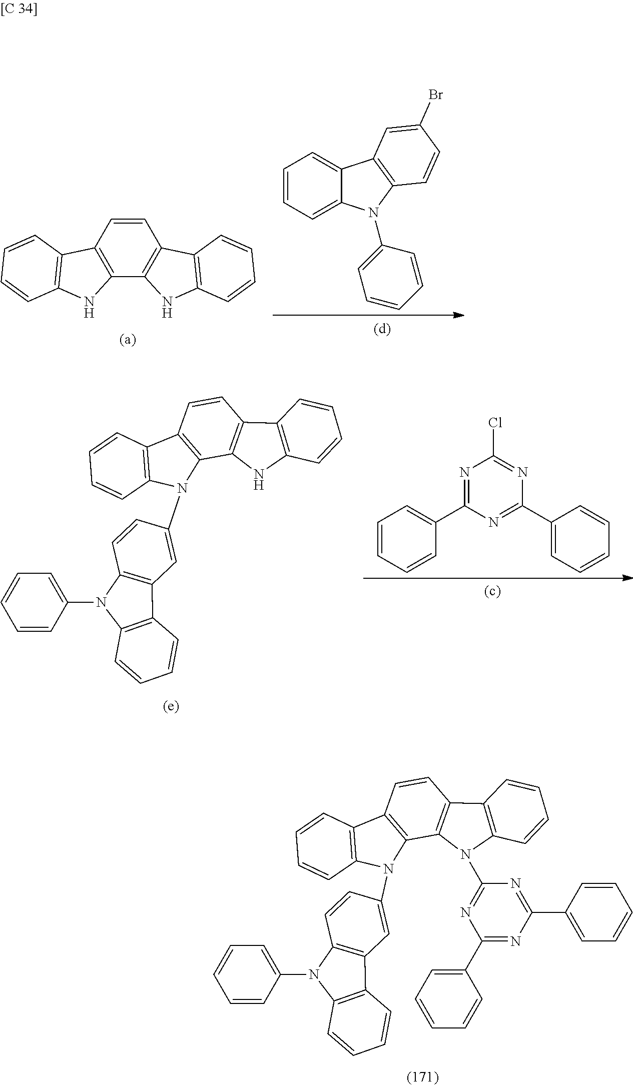 [SMILES: c1ccc2c(c1)[nH]c1c2ccc2c3ccccc3[nH]c21 ; Brc1ccc2c(c1)c1ccccc1n2-c1ccccc1 ; c1ccc(-n2c3ccccc3c3cc(-n4c5ccccc5c5ccc6c7ccccc7[nH]c6c54)ccc32)cc1 ; Clc1nc(-c2ccccc2)nc(-c2ccccc2)n1 ; c1ccc(-c2nc(-c3ccccc3)nc(-n3c4ccccc4c4ccc5c6ccccc6n(-c6ccc7c(c6)c6ccccc6n7-c6ccccc6)c5c43)n2)cc1]

To 20 g of compound (a) were added 31 g of compound (b), 1.5 g of copper iodide, 22 g of potassium carbonate, 0.06 g of 18-crown-6-ether, and 90 ml of 1,3-dimethyl-2-imidazolidinone, and stirred at 190° C. under a nitrogen atmosphere for 17 hours. After cooling to room temperature, the resultant was purified by silica gel chromatography and purified by crystallization to give 25 g (65% yield) of intermediate (e) as a white solid. Under a nitrogen atmosphere, 2.1 g of 61 wt % sodium hydride was added to 30 ml of N,N′-dimethylacetamide (DMAc) to prepare a suspension. Thereto was added 20 g of intermediate (e) dissolved in 100 mL of DMAc, and stirred for 30 minutes. Thereto was added 13 g of compound (c), and then stirred for 4 hours. The reaction solution was added to a mixture solution of methanol (110 ml) and distilled water (60 ml) while being stirred, and the resulting precipitated solid was collected by filtration. The resulting solid was purified by silica gel chromatography and purified by crystallization to give 9.1 g (62% yield) of compound (171) as a yellow solid (APCI-TOFMS, m/z 729[M+H]⁺).

### Synthesis Example 3

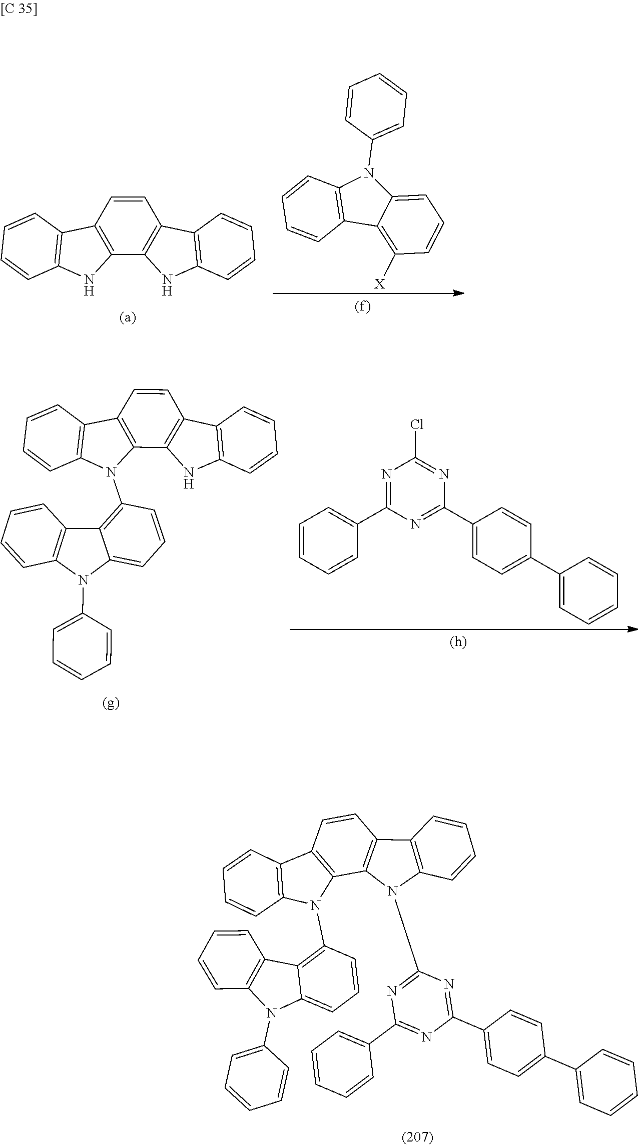

To 10 g of compound (a) were added 11 g of compound (f), 29 g of tripotassium phosphate, and 50 ml of 1,3-dimethyl-2-imidazolidinone, and stirred at 200° C. under a nitrogen atmosphere for 48 hours. After cooling to room temperature, the resultant was purified by silica gel chromatography and purified by crystallization to give 13.8 g (73% yield) of intermediate (g) as a white solid.

Under a nitrogen atmosphere, 1.1 g of 61 wt sodium hydride was added to 16 ml of N,N′-dimethylacetamide to prepare a suspension. Thereto was added 10 g of intermediate (g) dissolved in 50 mL of N,N′-dimethylacetamide, and stirred for 30 minutes. Thereto was added 8.5 g of compound (h), and then stirred for 6 hours. The reaction solution was added to a mixture solution of methanol (60 ml) and distilled water (30 ml) while being stirred, and the resulting precipitated solid was collected by filtration. The resulting solid was purified by silica gel chromatography and purified by crystallization to give 11 g (67% yield) of compound (207) as a yellow solid (APCI-TOFMS, m/z 805[M+H]+).

### Synthesis Example 4

Compound 601 was synthesized in accordance with the next reaction formula.

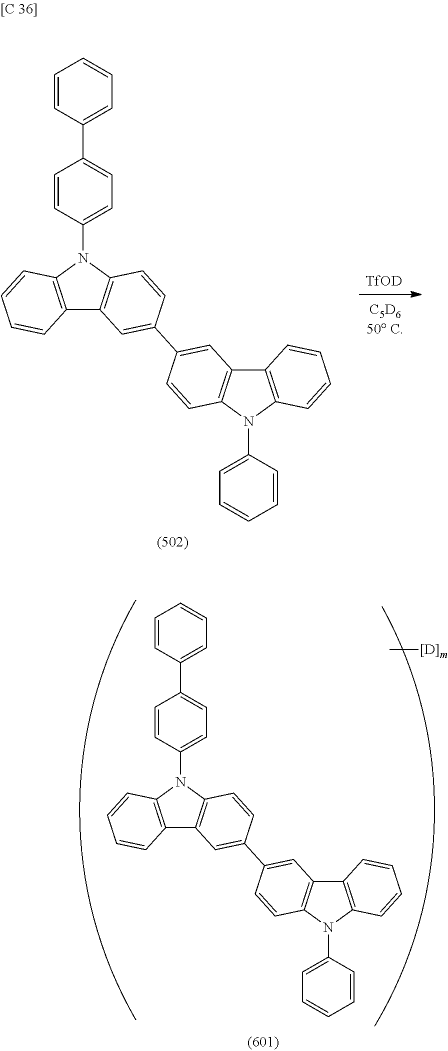

To 8.3 g (14.8 mmol) of compound (502) were added 160 mL of deuterated benzene (C₆D₆) and 10.0 g of deuterated trifluoromethanesulfonic acid (TfOD), and heated and stirred at 50° C. under a nitrogen atmosphere for 6.5 hours. A reaction liquid was added to a deuterium aqueous solution (200 mL) of sodium carbonate (7.4 g) and rapidly cooled, and separated and purified to give 2.5 g (4.25 mmol, 29% yield) of white solid compound (601) (APCI-TOFMS, m/z 589[M+H]+).

### Synthesis Example 5

Compound 605 was synthesized in accordance with the next reaction formula.

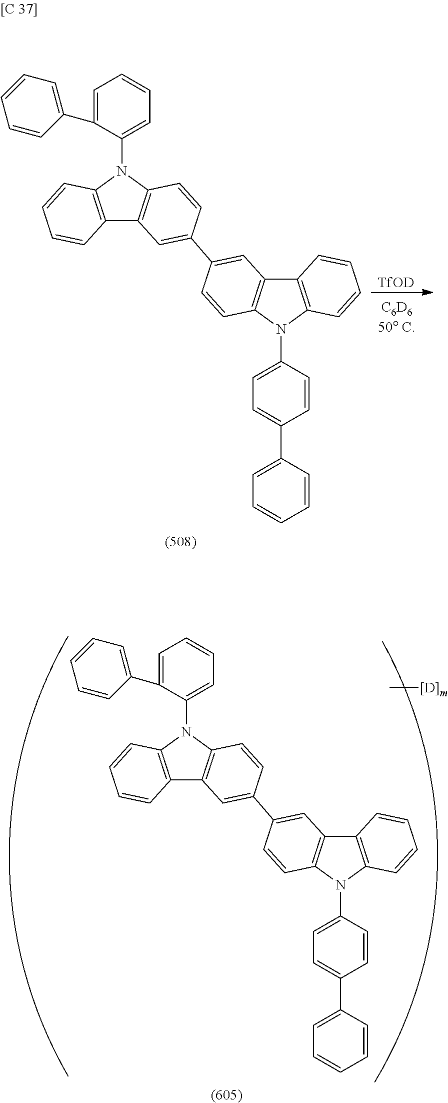

To 8.3 g of compound (508) were added 160 ml of deuterated benzene (C₆D₆) and 10.0 g of deuterated trifluoromethanesulfonic acid (TfOD), and heated and stirred at 50° C. under a nitrogen atmosphere for 6.5 hours. A reaction liquid was added to a deuterium aqueous solution (200 ml) of sodium carbonate (7.4 g) and rapidly cooled, and separated and purified to give 2.5 g of white solid compound (605) as a deuteride.

The average rate of deuteration in compound 601 obtained in Synthesis Example 4 above was determined by a proton nuclear magnetic resonance method. A measurement sample was prepared by dissolving compound 601 (5.0 mg) and dimethylsulfone (2.0 mg) as an internal standard material in deuterated tetrahydrofuran (1.0 ml). The average proton concentration [mol/g] of compound 601 included in the measurement sample was calculated from the ratio between integrated intensities derived from the internal standard material and compound 601. The average proton concentration [mol/g] of a non-deuterated substance (compound 502) of compound 601 was also calculated in the same manner. Next, the average rate of deuteration in compound 601 was calculated to be 74.0% by calculating the ratio between the proton concentration of compound 601 and the proton concentration of compound 502, and subtracting the ratio from 1. The average rate of deuteration in compound 605 obtained in Synthesis Example 5 was also calculated in the same manner. Compound 607 was synthesized and the average rate of deuteration therein was calculated in the same manner as in Synthesis Example 4 and Synthesis Example 5. The results are collectively shown in Table 1.

### Synthesis Example 6

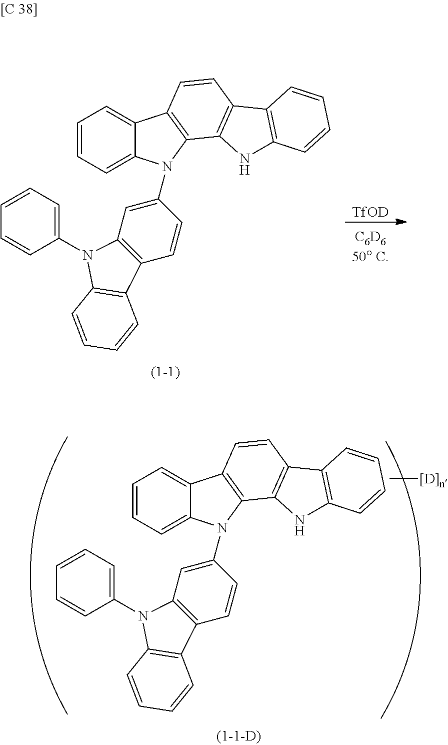

To 10 g of compound (1-1) were added 200 ml of deuterated benzene (C6D6) and 12.7 g of deuterated trifluoromethanesulfonic acid (TfOD), and heated and stirred at 50° C. under a nitrogen atmosphere for 4 hours. A reaction liquid was added to a deuterium aqueous solution (130 ml) of sodium carbonate (10.2 g) and rapidly cooled, and separated and purified to give 8.9 g of compound (1-1-D) as a deuteride.

### Synthesis Example 7

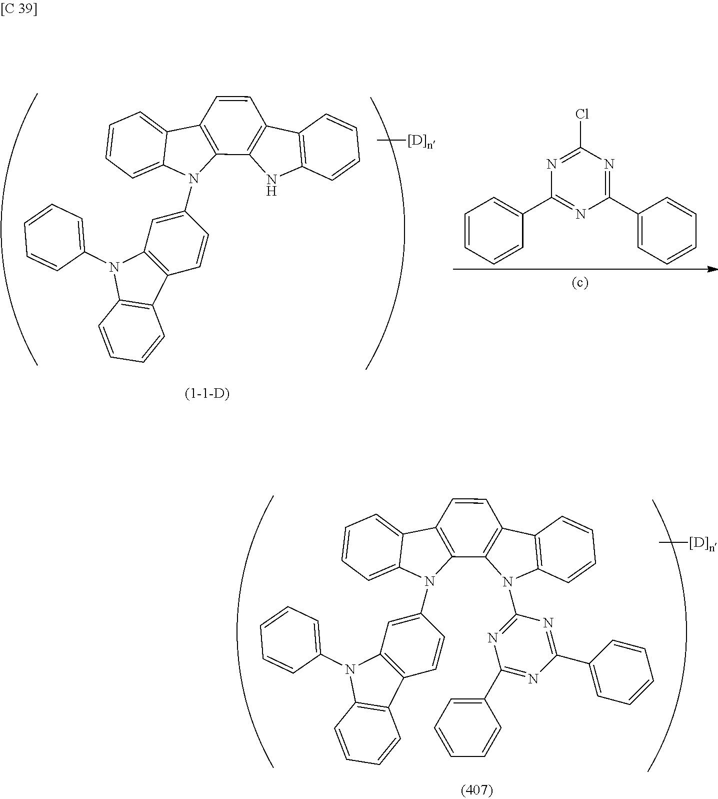

Under a nitrogen atmosphere, 1.8 g of 61 wt % sodium hydride was added to 50 ml of N,N′-dimethylacetamide to prepare a suspension. Thereto was added 8.9 g of intermediate (1-1-D) dissolved in 260 mL of N,N′-dimethylacetamide, and stirred for 30 minutes. Thereto was added 5.1 g of compound (c), and then stirred for 6 hours. The reaction solution was added to a mixture solution of methanol (300 ml) and distilled water (120 ml) while being stirred, and the resulting precipitated solid was collected by filtration. The resulting solid was purified by silica gel chromatography and purified by crystallization to give 7.7 g (66% yield) of compound 407 as a yellow solid.

The average rate of deuteration in compound 407 was determined by a proton nuclear magnetic resonance method. A measurement sample was prepared by dissolving compound 136 (5.0 mg) and dimethylsulfone (2.0 mg) as an internal standard material in deuterated tetrahydrofuran (1.0 ml). The average proton concentration [mol/g] of compound 407 included in the measurement sample was calculated from the ratio between integrated intensities derived from the internal standard material and compound 407. The average proton concentration [mol/g] of a non-deuterated substance (compound 136) of compound 407 was also calculated in the same manner. Next, the average rate of deuteration in compound 407 was calculated to be 52.1% by calculating the ratio between the proton concentration of compound 407 and the proton concentration of compound 136, and subtracting the ratio from 1.

Compounds 413 and 420 as deuterides were synthesized by reaction performed in the same manner as in Synthesis Examples 4 to 7. The average rate of deuteration in each of these compounds was calculated in the same manner as in 601 and 407. The results are shown in Table 1.

TABLE 1

| | Average rate of |
|---|---|
| Compound | deuteration |
| 601 | 74.0% |
| 605 | 68.8% |
| 607 | 68.6% |
| 407 | 52.1% |
| 413 | 48.2% |
| 420 | 50.5% |

Compounds used in Examples and Comparative Examples are shown below.

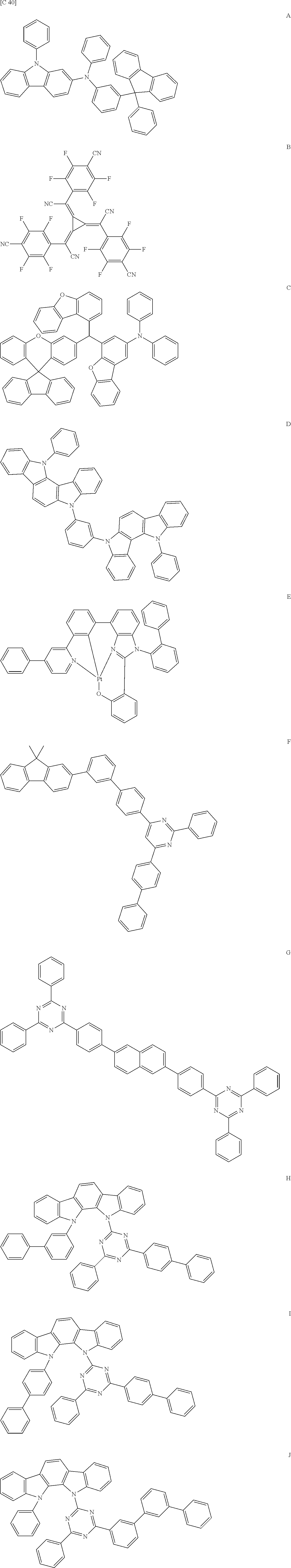

### Example 1

On a glass substrate on which an anode made of ITO with a film thickness of 110 nm was formed, respective thin films were laminated by a vacuum evaporation method at a degree of vacuum of 4.0×10⁻⁵ Pa.

First, compound A and compound B were co-vapordeposited as hole injection layers respectively from different vapor deposition sources and thus formed with a thickness of 10 nm on ITO. The co-vapor deposition was performed under vapor deposition conditions so that the concentration of compound B was 3 wt %.

Next, compound A was formed with a thickness of 110 nm as a first hole transport layer.

Next, compound C was formed with a thickness of 10 nm as a second hole transport layer.

Next, compound D was formed with a thickness of 5 nm as an electron blocking layer.

Next, a premixture obtained by weighing compound 136 (first host) and compound 502 (second host) as hosts at a weight ratio shown in Table 2, and mixing them while grinding in a mortar was evaporated from a single vapor deposition source, compound E was evaporated as a light-emitting dopant from a different vapor deposition source, and these were co-vapordeposited and formed with a thickness of 40 nm as a light-emitting layer. The co-vapor deposition was here performed under vapor deposition conditions so that the concentration of compound E was 15 wt % and the weight ratio between the first host and the second host was 30:70.

Next, compound F was formed with a thickness of 5 nm as a hole blocking layer.

Next, compound G was formed with a thickness of 30 nm as an electron transport layer.

Further, LiF was formed with a thickness of 1 nm as an electron injection layer on the electron transport layer.

Finally, Al was formed with a thickness of 70 nm as a cathode on the electron injection layer to produce an organic EL device.

### Examples 2 to 27 and Comparative Examples 1 to 9

Organic EL devices were produced in the same manner as in Example 1 except that compounds shown in Tables 2 and 3 were used as the first host and the second host at a weight ratio shown in Tables 2 and 3.

Evaluation results of the produced organic EL devices are shown in Tables 2 and 3. In the tables, the luminance, voltage, and current efficiency are values at a driving current of 10 mA/cm², and they exhibit initial characteristics. LT70 is a time period needed for driving at an initial luminance of 9000 nits and reduction of the luminance to 70%, and it represents lifetime characteristics. The numbers with which the first host and the second host are marked are numbers with which the exemplified compounds are marked, and the weight ratio represents first host second host. Herein, all the values of characteristics are expressed as relative values under the assumption that those of characteristics in Comparative Example 1 are each 103.

TABLE 2

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Example 1 | 136 | 502 | 30:70 | 101% | 111% | 106% |
| Example 2 | 136 | 502 | 40:60 | 98% | 116% | 110% |
| Example 3 | 136 | 502 | 50:50 | 97% | 117% | 112% |
| Example 4 | 136 | 601 | 30:70 | 102% | 111% | 123% |
| Example 5 | 136 | 601 | 40:60 | 98% | 117% | 133% |
| Example 6 | 136 | 601 | 50:50 | 96% | 118% | 132% |
| Example 7 | 407 | 601 | 30:70 | 101% | 112% | 131% |
| Example 8 | 407 | 601 | 40:60 | 98% | 117% | 141% |
| Example 9 | 407 | 601 | 50:50 | 97% | 120% | 150% |
| Example 10 | 171 | 502 | 30:70 | 100% | 117% | 107% |
| Example 11 | 171 | 502 | 40:60 | 100% | 117% | 109% |
| Example 12 | 171 | 502 | 50:50 | 98% | 117% | 112% |
| Example 13 | 171 | 601 | 30:70 | 101% | 113% | 114% |
| Example 14 | 171 | 601 | 40:60 | 99% | 119% | 125% |
| Example 15 | 171 | 601 | 50:50 | 97% | 118% | 135% |
| Example 16 | 413 | 601 | 30:70 | 101% | 113% | 136% |
| Example 17 | 413 | 601 | 40:60 | 99% | 118% | 143% |
| Example 18 | 413 | 601 | 50:50 | 98% | 120% | 152% |
| Example 19 | 207 | 508 | 30:70 | 100% | 110% | 107% |
| Example 20 | 207 | 508 | 40:60 | 98% | 111% | 110% |
| Example 21 | 207 | 508 | 50:50 | 94% | 112% | 114% |
| Example 22 | 207 | 605 | 30:70 | 100% | 110% | 108% |
| Example 23 | 207 | 605 | 40:60 | 98% | 114% | 117% |
| Example 24 | 207 | 605 | 50:50 | 96% | 112% | 126% |
| Example 25 | 420 | 605 | 30:70 | 102% | 116% | 127% |
| Example 26 | 420 | 605 | 40:60 | 98% | 119% | 139% |
| Example 27 | 420 | 605 | 50:50 | 97% | 117% | 146% |

TABLE 3

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Comparative | H | 502 | 30:70 | 4.1 | 65.0 | 556 |
| Example 1 | | | | | | |
| Comparative | H | 502 | 40:60 | 96% | 103% | 101% |
| Example 2 | | | | | | |
| Comparative | H | 502 | 50:50 | 95% | 105% | 104% |
| Example 3 | | | | | | |
| Comparative | I | 508 | 30:70 | 103% | 102% | 86% |
| Example 4 | | | | | | |
| Comparative | I | 508 | 40:60 | 101% | 103% | 95% |
| Example 5 | | | | | | |
| Comparative | I | 508 | 50:50 | 100% | 105% | 65% |
| Example 6 | | | | | | |
| Comparative | J | 508 | 30:70 | 111% | 101% | 72% |
| Example 7 | | | | | | |
| Comparative | J | 508 | 40:60 | 106% | 104% | 77% |
| Example 8 | | | | | | |
| Comparative | J | 508 | 50:50 | 105% | 104% | 55% |
| Example 9 | | | | | | |

### Examples 28 to 54 and Comparative Examples 10 to 18

Organic EL devices were produced in the same manner as in Example 1 except that compounds shown in Tables 4 and 5 were used as the first host and the second host and co-vapordeposited respectively from different vapor deposition sources at a weight ratio shown in Tables 4 and 5.

The organic EL devices obtained were evaluated in the same manner as in Example 1 and the like above. The results are shown in Tables 4 and 5. Herein, all the values of characteristics are expressed as relative values under the assumption that those of characteristics in Comparative Example 10 are each 100%.

TABLE 4

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Example 28 | 207 | 502 | 30:70 | 96% | 110% | 111% |
| Example 29 | 207 | 502 | 40:60 | 96% | 110% | 111% |
| Example 30 | 207 | 502 | 50:50 | 92% | 112% | 114% |
| Example 31 | 207 | 601 | 30:70 | 98% | 110% | 118% |
| Example 32 | 207 | 601 | 40:60 | 96% | 110% | 128% |
| Example 33 | 207 | 601 | 50:50 | 98% | 112% | 130% |
| Example 34 | 420 | 601 | 30:70 | 99% | 110% | 139% |
| Example 35 | 420 | 601 | 40:60 | 97% | 115% | 147% |
| Example 36 | 420 | 601 | 50:50 | 99% | 118% | 158% |
| Example 37 | 136 | 508 | 30:70 | 99% | 110% | 107% |
| Example 38 | 136 | 508 | 40:60 | 98% | 113% | 109% |
| Example 39 | 136 | 508 | 50:50 | 97% | 117% | 111% |
| Example 40 | 136 | 605 | 30:70 | 101% | 110% | 110% |
| Example 41 | 136 | 605 | 40:60 | 98% | 115% | 116% |
| Example 42 | 136 | 605 | 50:50 | 96% | 119% | 126% |
| Example 43 | 407 | 605 | 30:70 | 101% | 110% | 122% |
| Example 44 | 407 | 605 | 40:60 | 98% | 114% | 133% |
| Example 45 | 407 | 605 | 50:50 | 96% | 118% | 138% |
| Example 46 | 171 | 508 | 30:70 | 100% | 111% | 108% |
| Example 47 | 171 | 508 | 40:60 | 99% | 114% | 110% |
| Example 48 | 171 | 508 | 50:50 | 97% | 120% | 115% |
| Example 49 | 171 | 605 | 30:70 | 104% | 111% | 111% |
| Example 50 | 171 | 605 | 40:60 | 102% | 115% | 118% |
| Example 51 | 171 | 605 | 50:50 | 99% | 122% | 125% |
| Example 52 | 413 | 605 | 30:70 | 104% | 113% | 122% |
| Example 53 | 413 | 605 | 40:60 | 101% | 118% | 133% |
| Example 54 | 413 | 605 | 50:50 | 99% | 121% | 135% |

TABLE 5

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Comparative | I | 502 | 30:70 | 4.1 | 65.8 | 557 |
| Example 10 | | | | | | |
| Comparative | I | 502 | 40:60 | 95% | 101% | 101% |
| Example 11 | | | | | | |
| Comparative | I | 502 | 50:50 | 93% | 103% | 103% |
| Example 12 | | | | | | |
| Comparative | H | 508 | 30:70 | 106% | 101% | 86% |
| Example 13 | | | | | | |
| Comparative | H | 508 | 40:60 | 103% | 104% | 95% |
| Example 14 | | | | | | |
| Comparative | H | 508 | 50:50 | 101% | 102% | 67% |
| Example 15 | | | | | | |
| Comparative | J | 502 | 30:70 | 106% | 101% | 88% |
| Example 16 | | | | | | |
| Comparative | J | 502 | 40:60 | 102% | 103% | 97% |
| Example 17 | | | | | | |
| Comparative | J | 502 | 50:50 | 103% | 102% | 86% |
| Example 18 | | | | | | |

### Examples 55 to 87 and Comparative Examples 19 to 27

Organic EL device were each produced in the same manner as in Example 1 except that a premixture obtained by using compounds shown in Tables 6 and 7 as a first host and a second host, weighting them at a weight ratio shown in Tables 6 and 7, mixing them while grinding in a mortar, placing the resulting product in a vacuum chamber, heating and melting the resulting product by heating to 320° C. in a state of a reduced pressure of 100 Pa or less, then cooling the resulting product to room temperature, and pulverizing and mixing the resulting product was vapordeposited from a single vapor deposition source.

The organic EL devices obtained were evaluated in the same manner as in Example 1 and the like above. The results are shown in Tables 6 and 7. Herein, all the values of characteristics are expressed as relative values under the assumption that those of characteristics in Comparative Example 19 are each 100%.

TABLE 6

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Example 55 | 136 | 502 | 30:70 | 102% | 111% | 109% |
| Example 56 | 136 | 502 | 40:60 | 99% | 117% | 112% |
| Example 57 | 136 | 502 | 50:50 | 97% | 118% | 114% |
| Example 58 | 136 | 601 | 30:70 | 101% | 112% | 121% |
| Example 59 | 136 | 601 | 40:60 | 99% | 117% | 131% |
| Example 60 | 136 | 601 | 50:50 | 95% | 118% | 132% |
| Example 61 | 407 | 601 | 30:70 | 101% | 111% | 133% |
| Example 62 | 407 | 601 | 40:60 | 97% | 118% | 145% |
| Example 63 | 407 | 601 | 50:50 | 95% | 119% | 151% |
| Example 64 | 136 | 607 | 30:70 | 105% | 114% | 133% |
| Example 65 | 136 | 607 | 40:60 | 102% | 119% | 145% |
| Example 66 | 136 | 607 | 50:50 | 100% | 120% | 149% |
| Example 67 | 407 | 607 | 30:70 | 104% | 115% | 146% |
| Example 68 | 407 | 607 | 40:60 | 102% | 119% | 156% |
| Example 69 | 407 | 607 | 50:50 | 99% | 120% | 160% |
| Example 70 | 171 | 502 | 30:70 | 101% | 115% | 109% |
| Example 71 | 171 | 502 | 40:60 | 100% | 118% | 112% |
| Example 72 | 171 | 502 | 50:50 | 98% | 117% | 113% |
| Example 73 | 171 | 601 | 30:70 | 101% | 112% | 114% |
| Example 74 | 171 | 601 | 40:60 | 98% | 120% | 123% |
| Example 75 | 171 | 601 | 50:50 | 96% | 117% | 130% |
| Example 76 | 413 | 601 | 30:70 | 102% | 113% | 132% |
| Example 77 | 413 | 601 | 40:60 | 100% | 118% | 138% |
| Example 78 | 413 | 601 | 50:50 | 98% | 120% | 145% |
| Example 79 | 207 | 508 | 30:70 | 101% | 110% | 110% |
| Example 80 | 207 | 508 | 40:60 | 99% | 112% | 112% |
| Example 81 | 207 | 508 | 50:50 | 96% | 114% | 115% |
| Example 82 | 207 | 605 | 30:70 | 99% | 110% | 109% |
| Example 83 | 207 | 605 | 40:60 | 97% | 114% | 115% |
| Example 84 | 207 | 605 | 50:50 | 96% | 113% | 128% |
| Example 85 | 420 | 605 | 30:70 | 101% | 117% | 125% |
| Example 86 | 420 | 605 | 40:60 | 98% | 120% | 142% |
| Example 87 | 420 | 605 | 50:50 | 96% | 115% | 145% |

TABLE 7

| | | | | | Current | |
|---|---|---|---|---|---|---|
| | | Second | | Volt- | effi- | |
| | First host | host | Mixing | age | ciency | LT70 |
| | compound | compound | ratio | (V) | (cd/A) | (h) |
| Comparative | H | 502 | 30:70 | 4.1 | 72.1 | 673 |
| Example 19 | | | | | | |
| Comparative | H | 502 | 40:60 | 97% | 102% | 101% |
| Example 20 | | | | | | |
| Comparative | H | 502 | 50:50 | 96% | 104% | 104% |
| Example 21 | | | | | | |
| Comparative | I | 508 | 30:70 | 102% | 102% | 86% |
| Example 22 | | | | | | |
| Comparative | I | 508 | 40:60 | 102% | 103% | 95% |
| Example 23 | | | | | | |
| Comparative | I | 508 | 50:50 | 100% | 104% | 65% |
| Example 24 | | | | | | |
| Comparative | J | 508 | 30:70 | 110% | 102% | 72% |
| Example 25 | | | | | | |
| Comparative | J | 508 | 40:60 | 107% | 104% | 77% |
| Example 26 | | | | | | |
| Comparative | J | 508 | 50:50 | 106% | 103% | 55% |
| Example 27 | | | | | | |

From the results, it is understood that Examples 1 to 87 not only had lifetime characteristics comparable to or better than those in Comparative Examples, but also were enhanced in efficiency and exhibited in favorable characteristics. In general, the efficiency and the lifetime may also be in a trade-off relationship and enhancements in both current efficiency and lifetime characteristics are difficult. In particular, an enhancement in current efficiency leads to a reduction in power consumption and an enhancement in luminance, and therefore a more practical device can be obtained if an enhancement in current efficiency can be achieved with certain lifetime characteristics being obtained. In view of this, Examples with the mixed composition according to the present invention, when compared with Comparative Examples 1, 10, and 19 with conventional compounds as some of mixed host materials, could not only keep lifetime characteristics at a proportion of about 105% or exhibit lifetime characteristics at a proportion of 110% or more, but also achieve a current efficiency of 110% or more depending on the combination of materials and the mixing ratio, and could provide organic EL devices advantageous for practical use.

Table 8 shows the 50% weight reduction temperatures (T50) of compounds 508, 605, 502, 601, 607, 136, 407, 171, 413, 207, 429, H, I, J, and K.

TABLE 8

| Compound | T50 [° C.] |
|---|---|
| 508 | 275 |
| 605 | 278 |
| 502 | 265 |
| 601 | 267 |
| 607 | 269 |
| 136 | 264 |
| 407 | 267 |
| 171 | 265 |
| 413 | 267 |
| 207 | 270 |
| 420 | 271 |
| H | 268 |
| I | 286 |
| J | 285 |

### REFERENCE SIGNS LIST

1 substrate, 2 anode, 3 hole injection layer, 4 hole transport layer, 5 light-emitting layer, 6 electron transport layer, 7 cathode.

1. A mixed composition comprising a compound represented by the following general formula (1) and a compound represented by the following general formula (2): 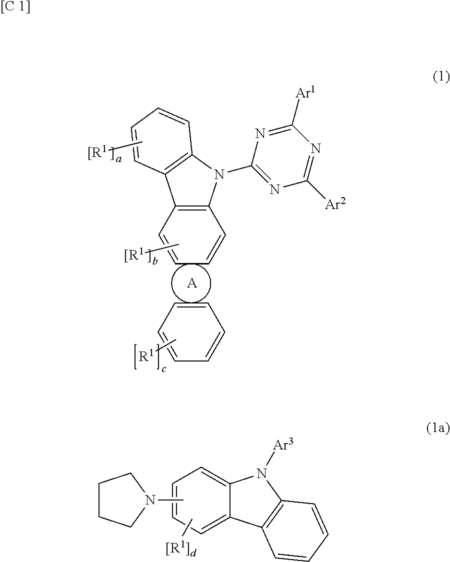
wherein a ring A is a 5-membered heterocycle fused to two adjacent rings at any positions and represented by formula (1a); Ar¹ and Ar² each independently represent a substituted or unsubstituted phenyl group, a substituted or unsubstituted biphenyl group, or a substituted or unsubstituted terphenyl group; Ar³ represents a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other, and aromatic hydrocarbon groups or aromatic heterocyclic groups in the case of these aromatic groups linked are the same as or different from each other; each R¹ independently represents a substituted or unsubstituted aromatic hydrocarbon group having 6 to 10 carbon atoms, a substituted or unsubstituted aromatic heterocyclic group having 3 to 12 carbon atoms, or a substituted or unsubstituted linked aromatic group in which two to five of these aromatic groups are linked to each other, and aromatic hydrocarbon groups or aromatic heterocyclic groups in the case of these aromatic groups linked are the same as or different from each other; and a to d represent the number of substitutions, a and c each independently represent an integer of 0 to 4, b represents an integer of 0 to 2, and d represents an integer of 0 to 3; 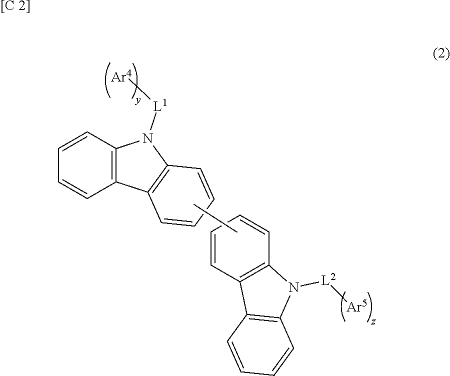
wherein Ar⁴ and Ar⁵ each independently represent a substituted or unsubstituted aromatic hydrocarbon group having 6 to 14 carbon atoms, or a substituted or unsubstituted linked aromatic group in which two of these aromatic hydrocarbon groups are linked to each other, and aromatic hydrocarbon groups in the case of these aromatic rings linked are the same as or different from each other; L¹ and L² each independently represent a direct bond, or a substituted or unsubstituted phenylene group; and y and z represent the number of substitutions and each independently represent an integer of 1 to 2.

2. The mixed composition according to claim 1, wherein the mixed composition comprises 20 wt % or more and 70 wt % or less of the compound represented by the general formula (1) based on the compound represented by the general formula (1) and the compound represented by the general formula (2) in total.

3. The mixed composition according to claim 1, wherein hydrogen in at least one compound of the compounds represented by the general formula (1) and the general formula (2) is partially or fully replaced by deuterium.

4. The mixed composition according to claim 1, wherein hydrogen in at least one compound of the compounds represented by the general formula (1) and the general formula (2) is partially or fully replaced by deuterium, and an average rate of deuteration is 30% or more.

5. The mixed composition according to claim 1, wherein the mixed composition is a premixture which is a material for producing at least one layer in an organic electroluminescent device by a vapor deposition method and which is formed by previous mixing before vapor deposition.

6. The mixed composition according to claim 1, wherein the mixed composition is a premixture which is a material for producing at least one layer in an organic electroluminescent device by a vapor deposition method and which is formed by previous mixing by heating and melting before vapor deposition.

7. The mixed composition according to claim 5, wherein a difference in 50% weight reduction temperatures of the compound represented by the general formula (1) and the compound represented by the general formula (2) is within 20° C.

8. An organic electroluminescent device comprising a plurality of organic layers between an anode and a cathode, wherein at least one of the organic layers contains the mixed composition according to claim 1.

9. The organic electroluminescent device according to claim 8, wherein the organic layer containing the mixed composition is at least one selected from a light-emitting layer, a hole injection layer, a hole transport layer, an electron transport layer, an electron injection layer, a hole blocking layer and an electron blocking layer.

10. The organic electroluminescent device according to claim 8, wherein the organic layer containing the mixed composition is a light-emitting layer, and comprises the compound represented by the general formula (1) as a first host and the compound represented by the general formula (2) as a second host, and the light-emitting layer contains at least one light-emitting dopant.

11. A method for producing an organic electroluminescent device comprising a plurality of organic layers including a light-emitting layer between an anode and a cathode, the method comprising the steps of: providing the mixed composition according to claim 5, and vapor-depositing the mixed composition from one vapor deposition source to form a light-emitting layer.

12. The mixed composition according to claim 1, wherein the compound represented by the general formula (1) is selected from the group consisting of the following compounds, wherein n represents the average number of replacements by deuterium (D) and is varied depending on the average rate of deuteration in each of the compounds.

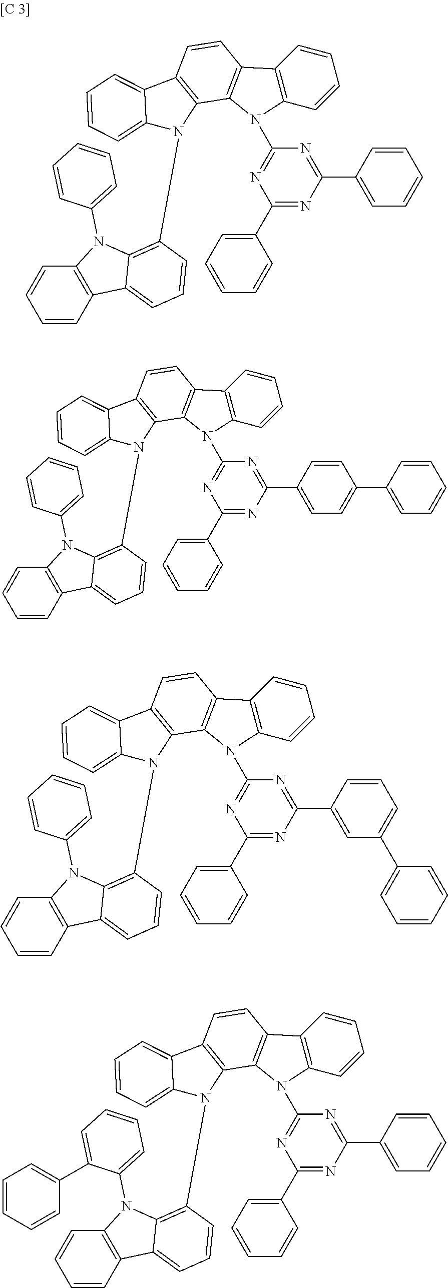

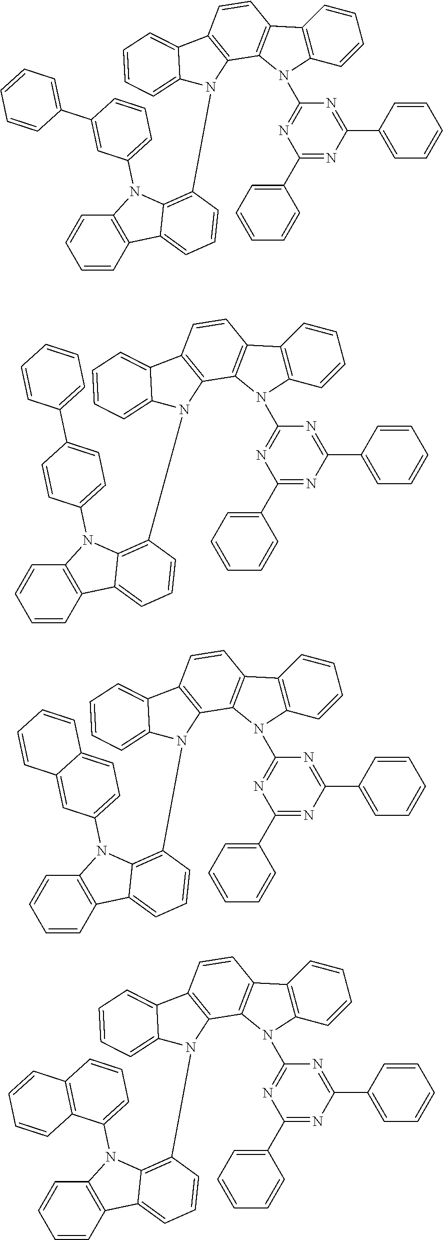

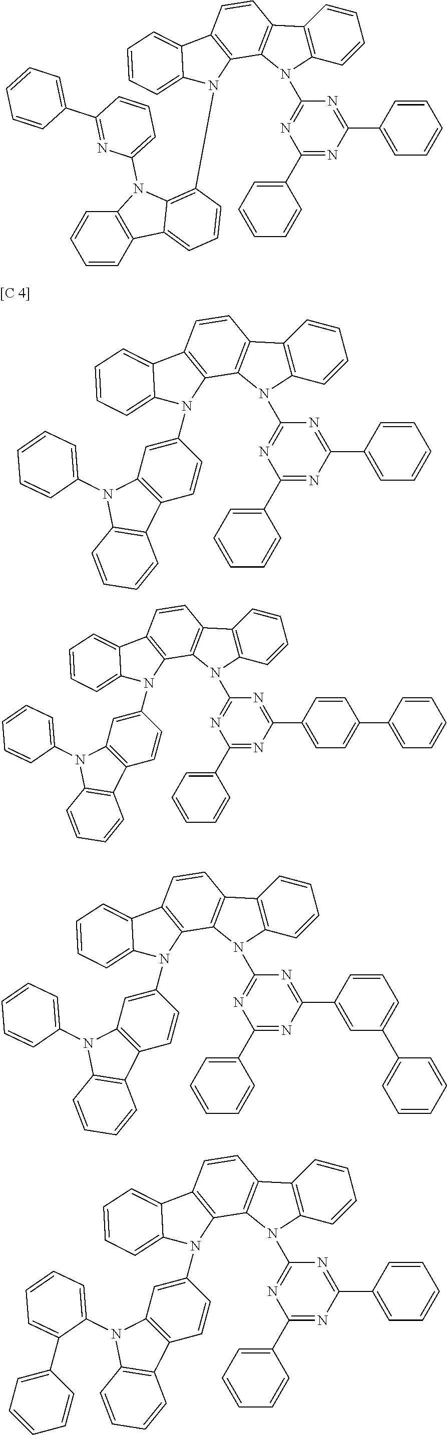

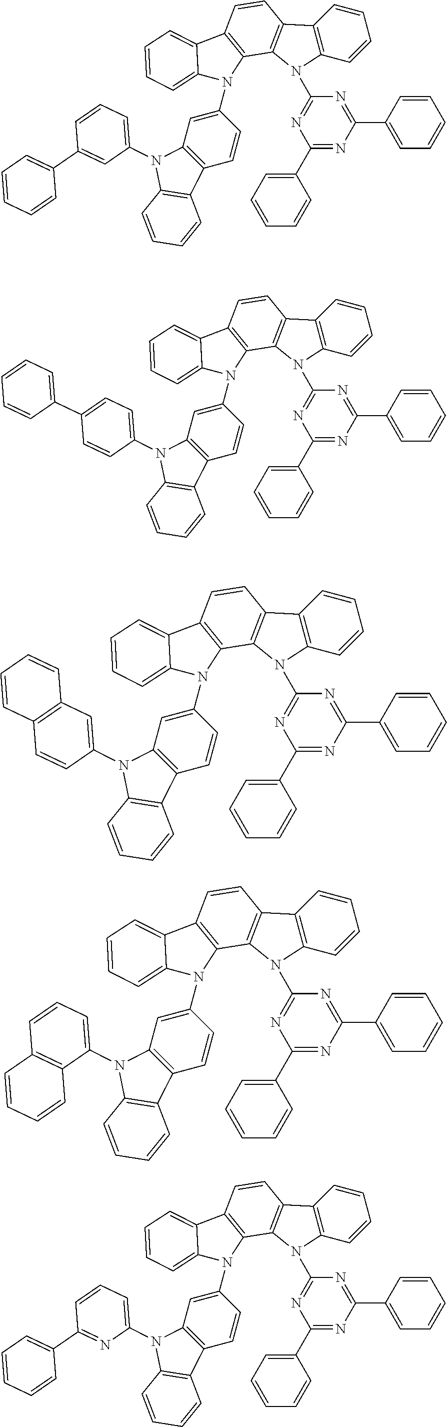

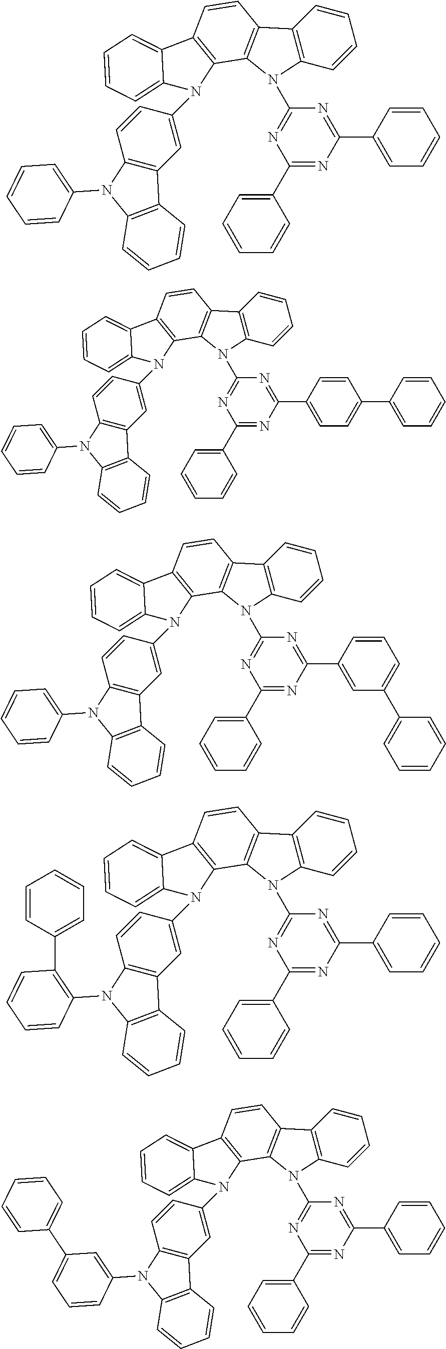

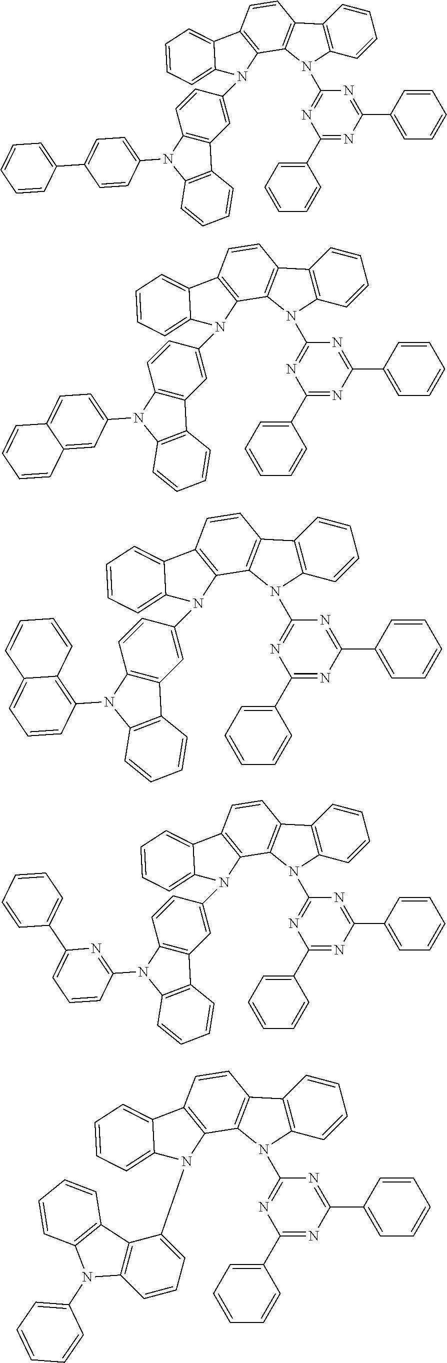

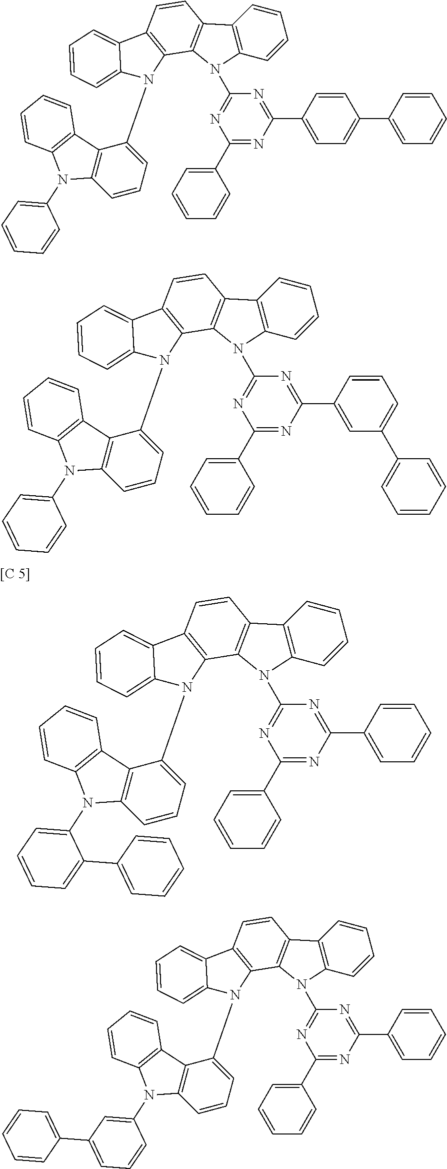

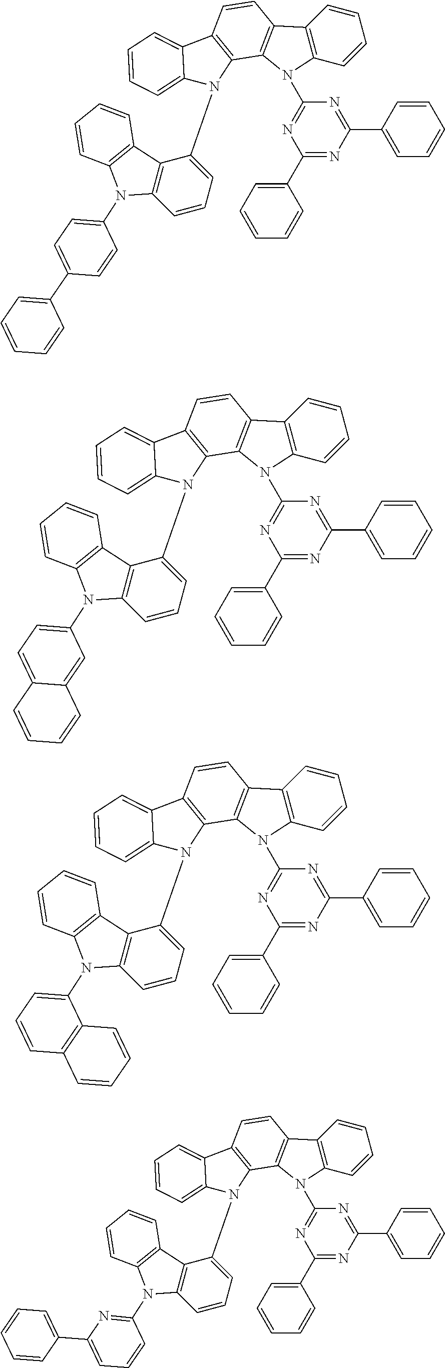

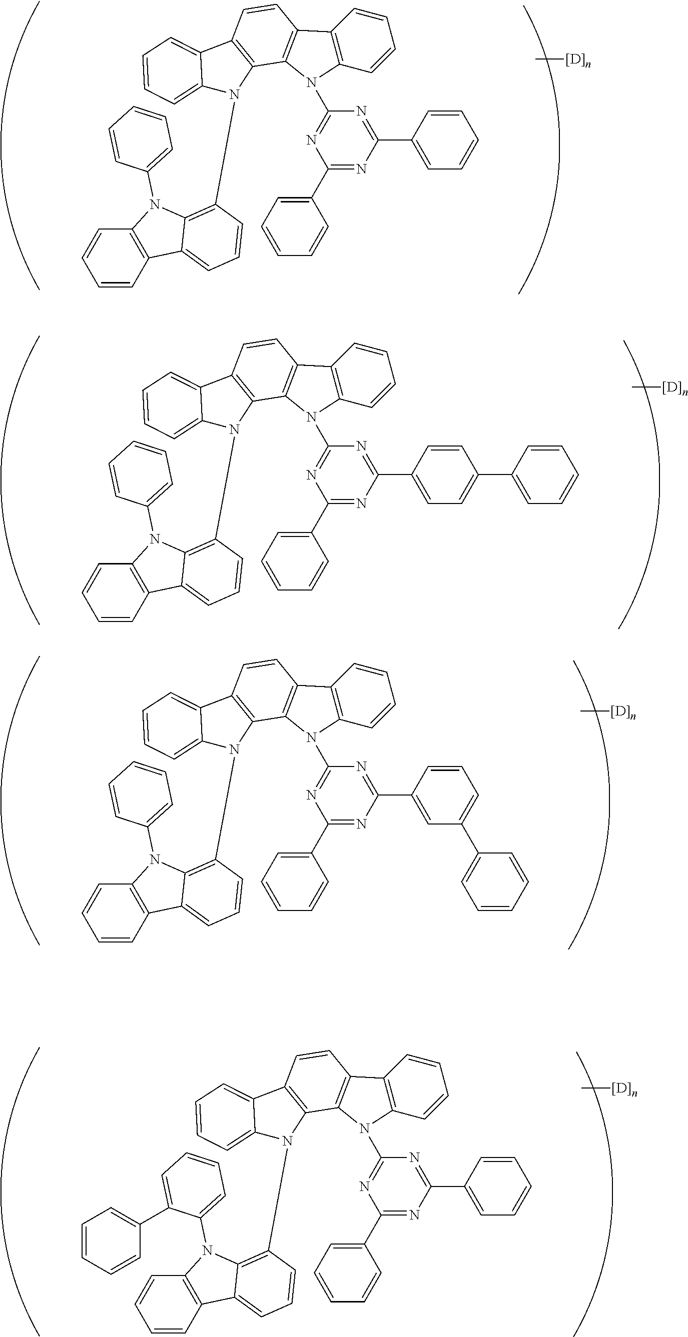

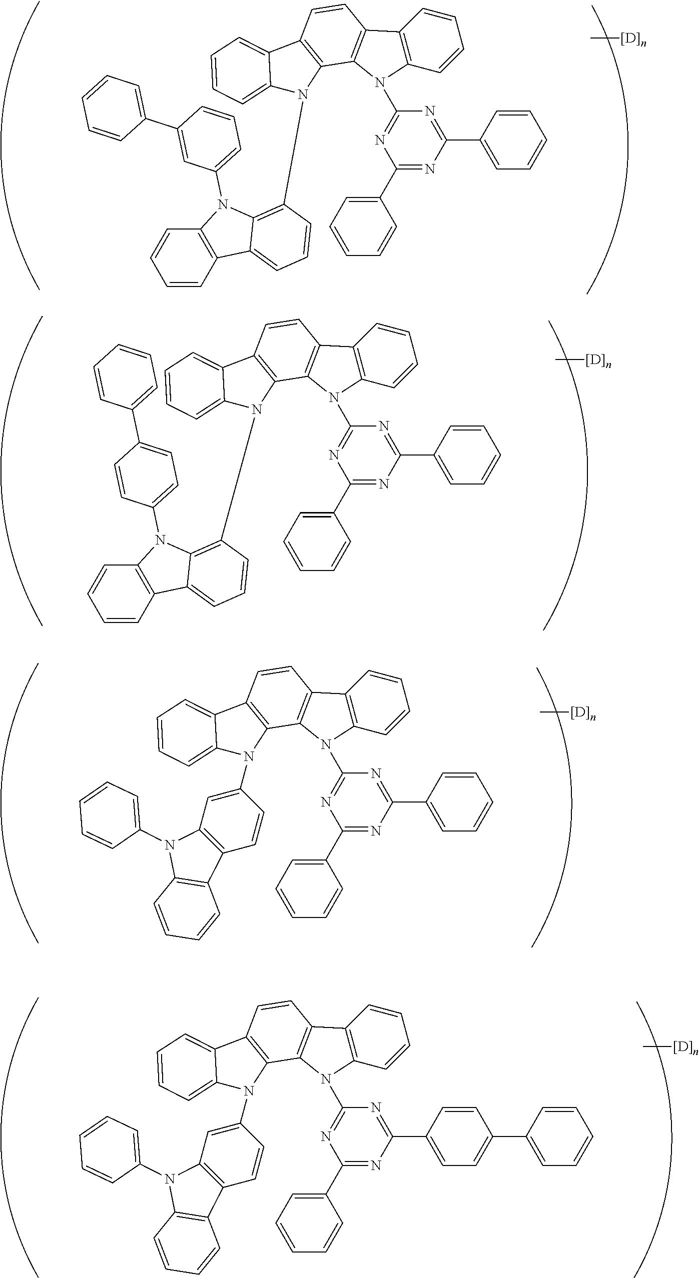

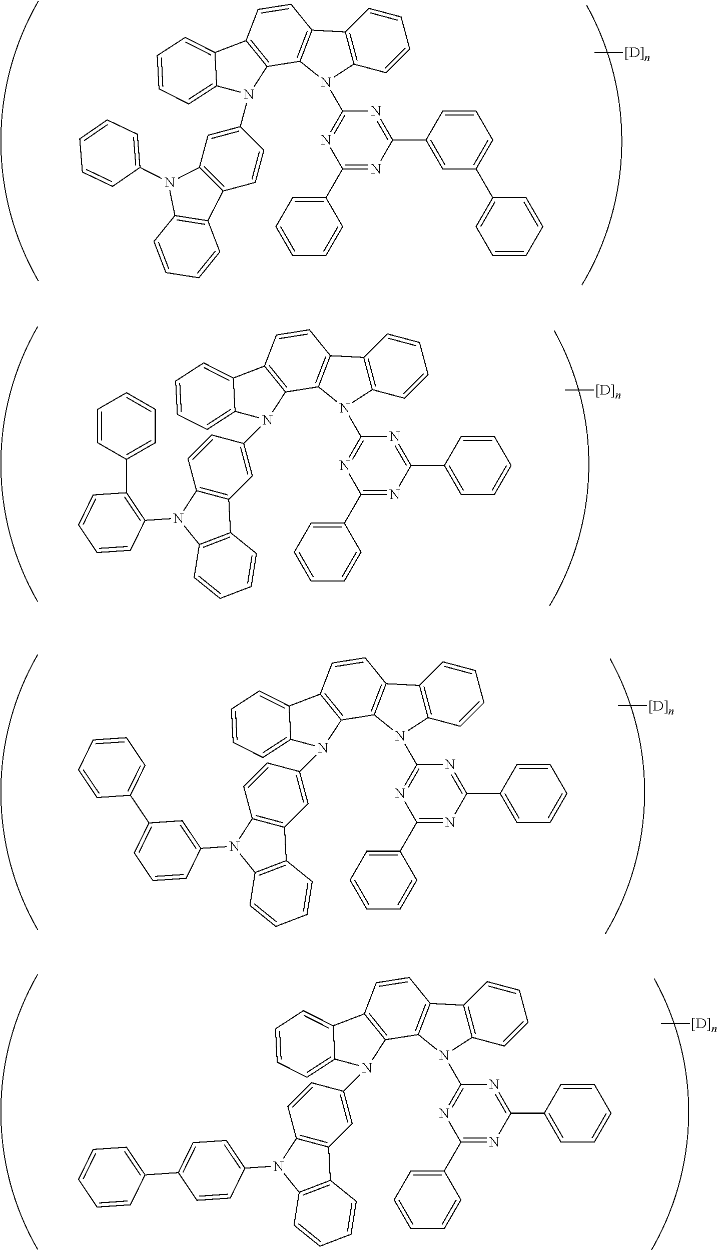

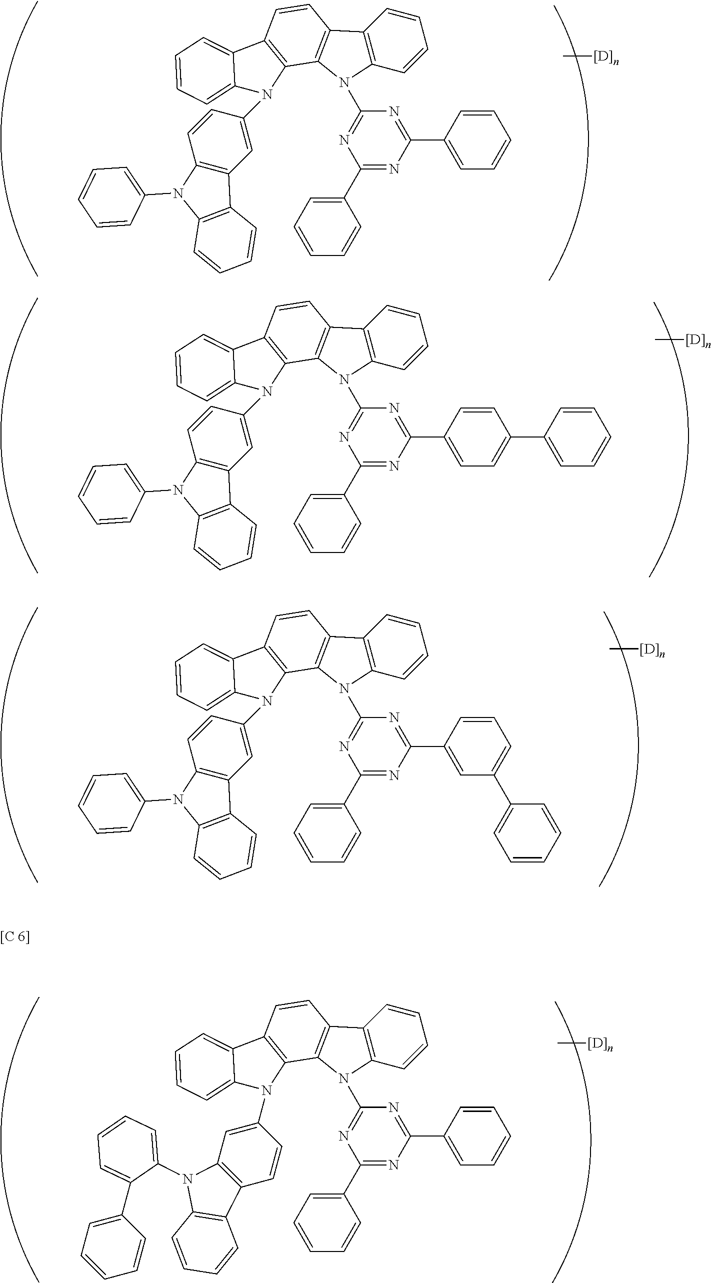

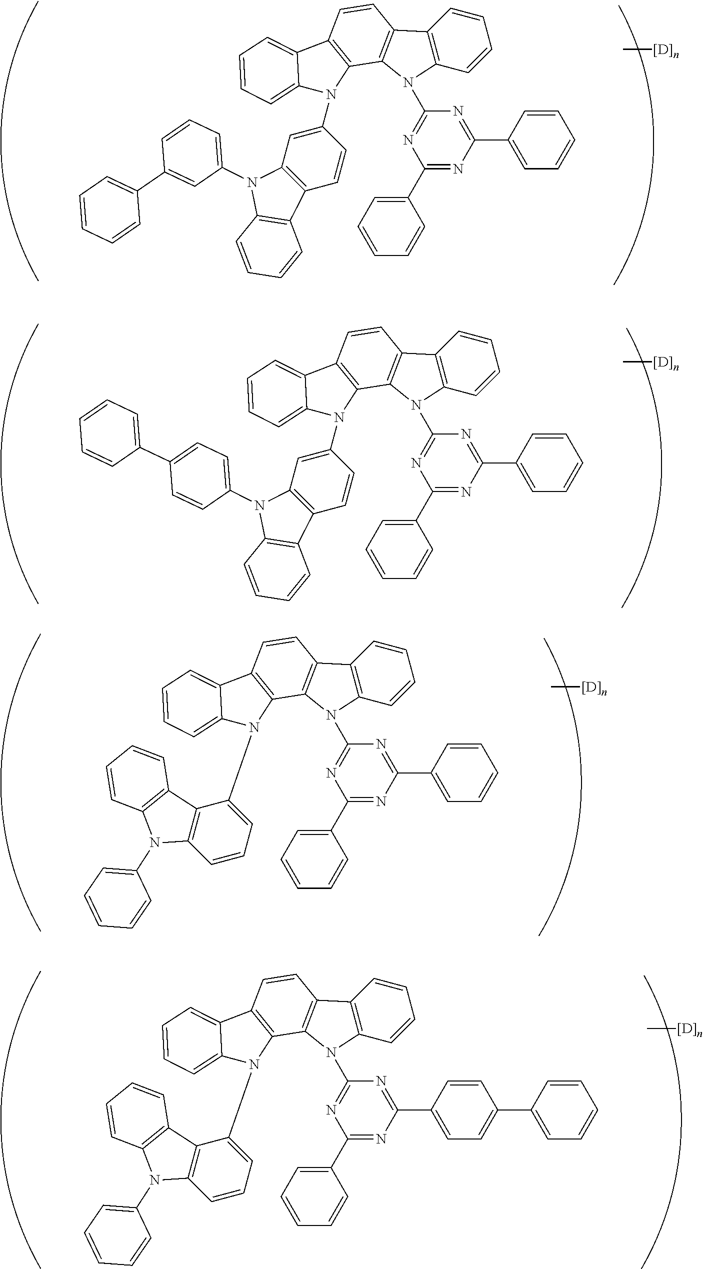

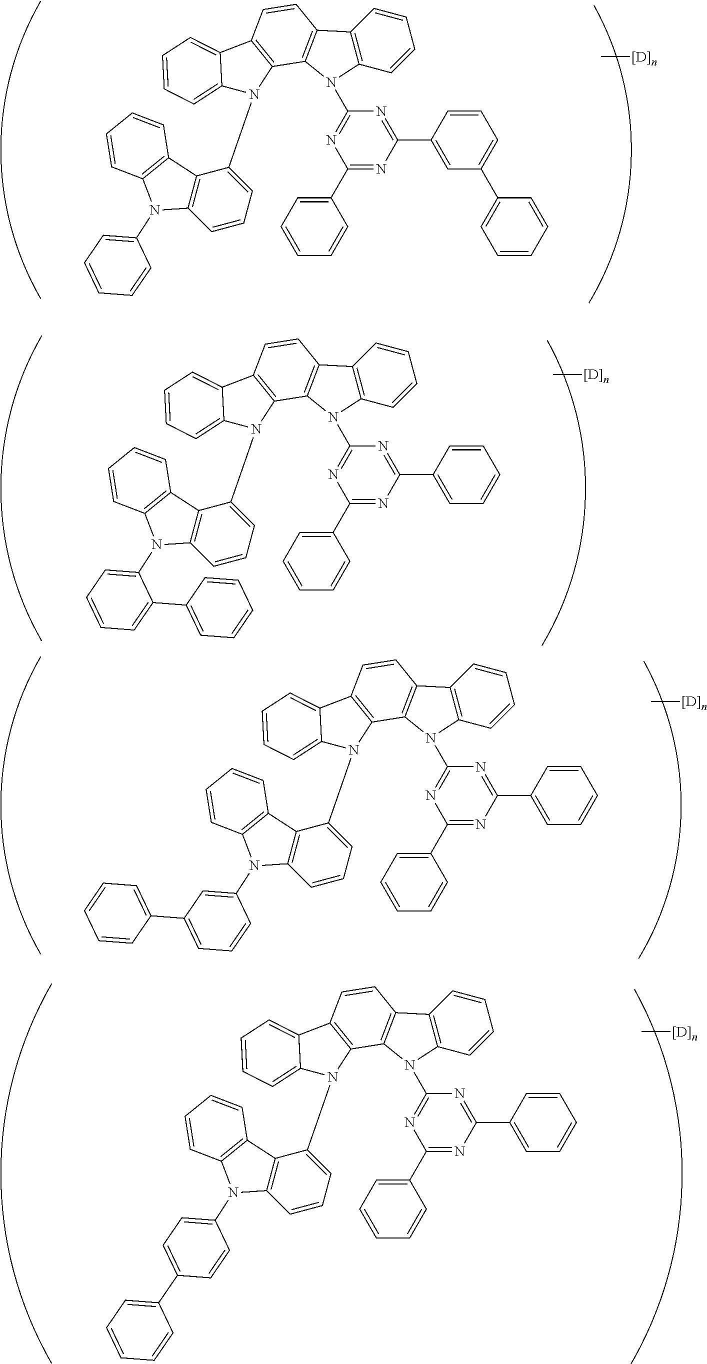

13. The mixed composition according to claim 1, wherein the compound represented by the general formula (2) is selected from the group consisting of the following compounds, wherein m represents the average number of replacements by deuterium (D) and is varied depending on the average rate of deuteration in each of the compounds.

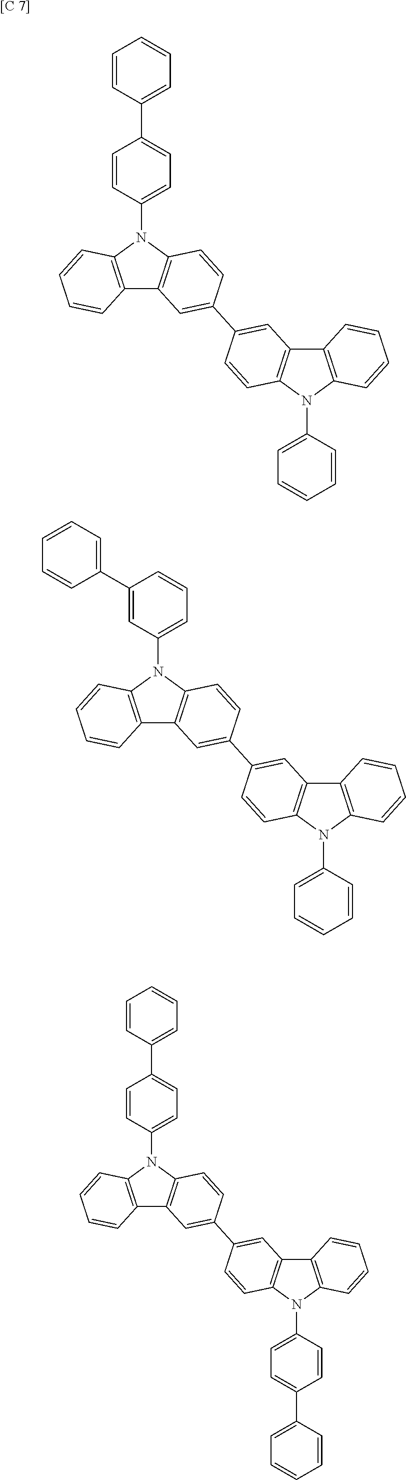

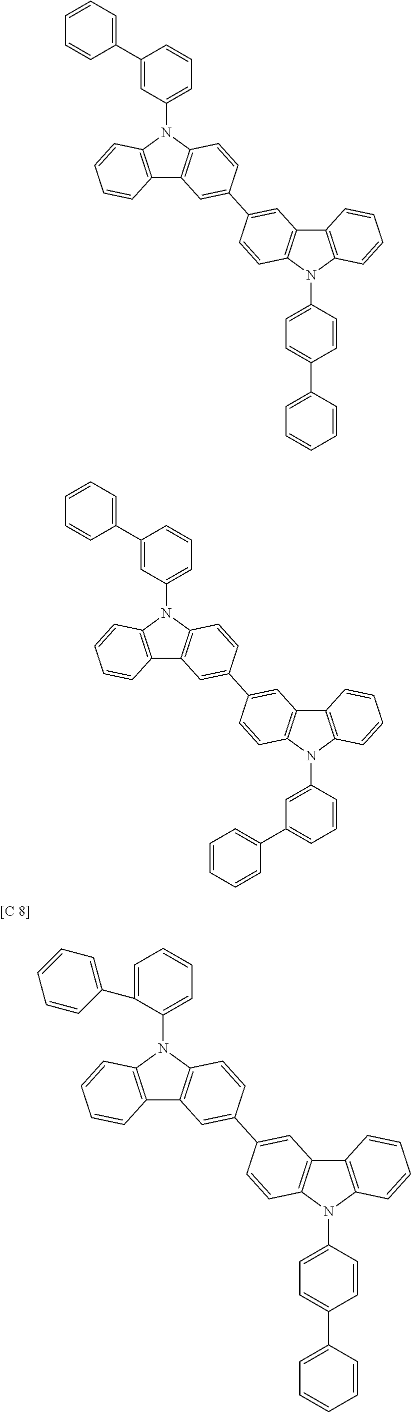

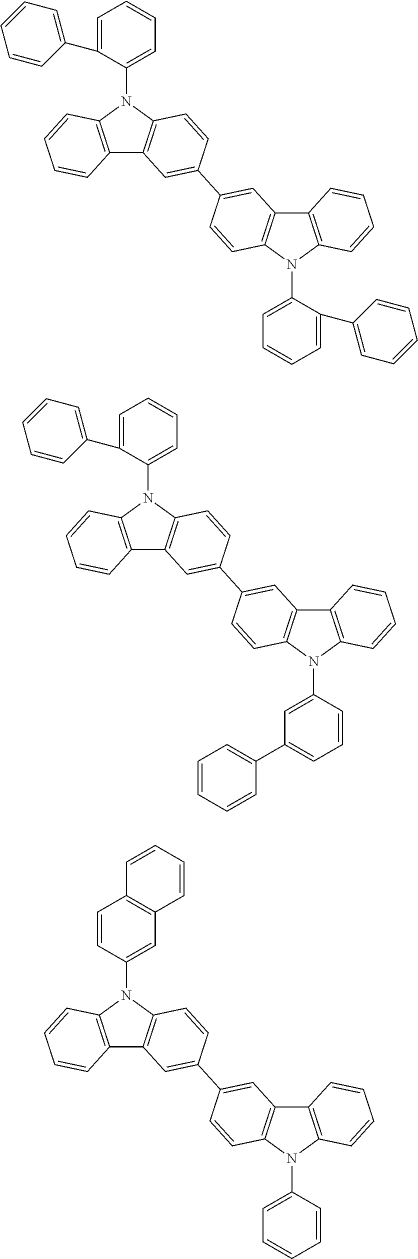

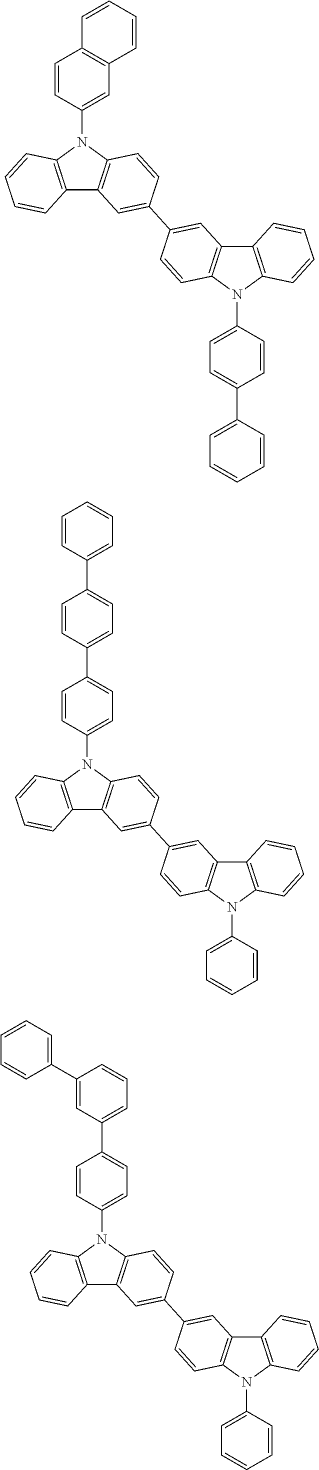

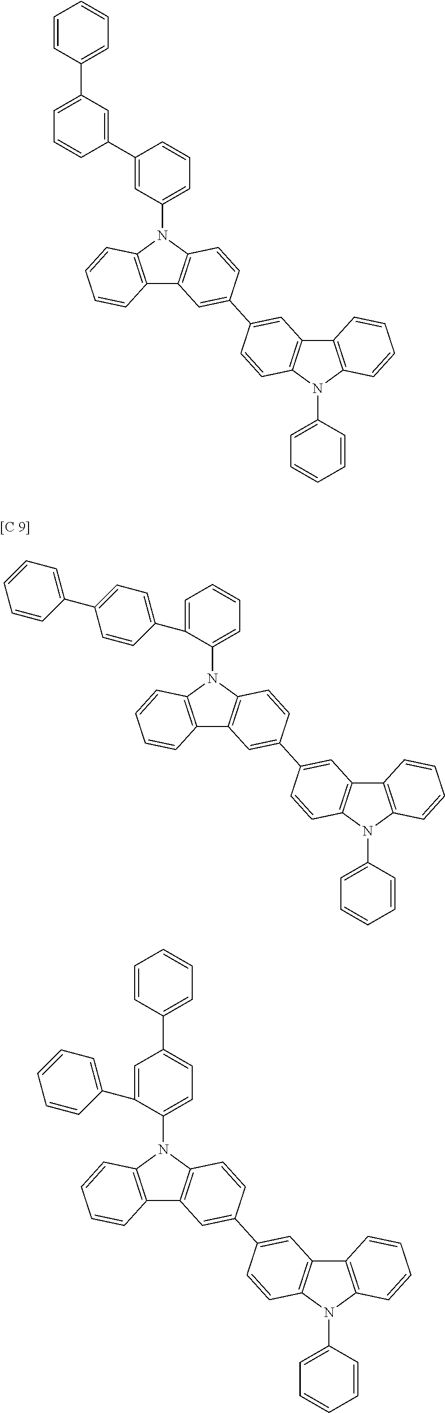

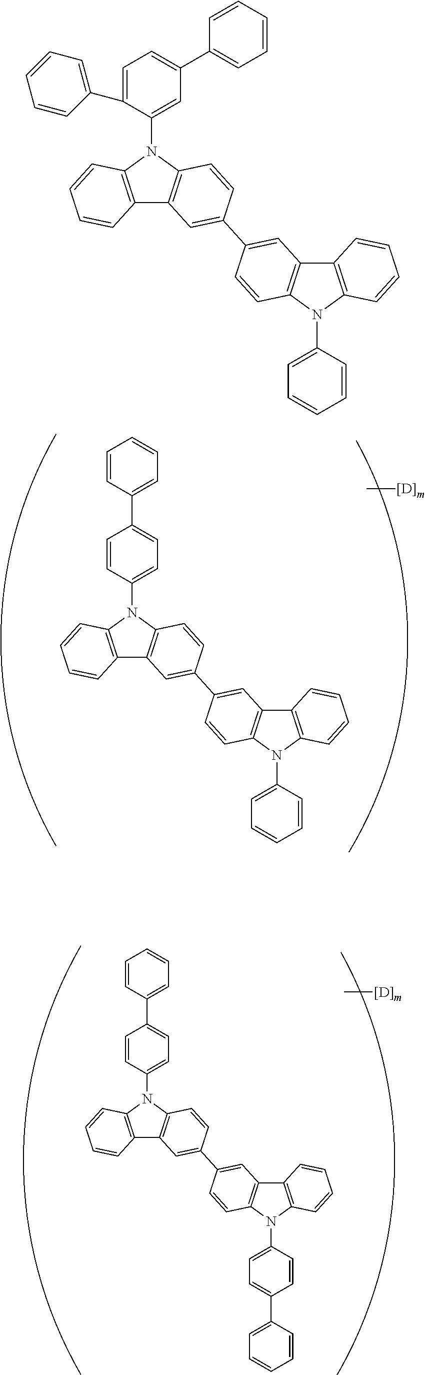

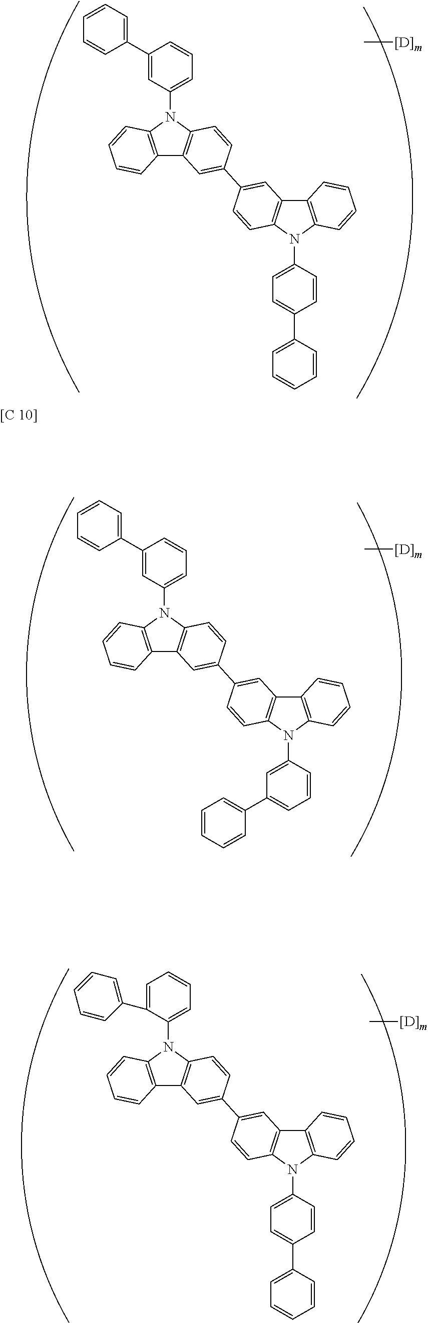

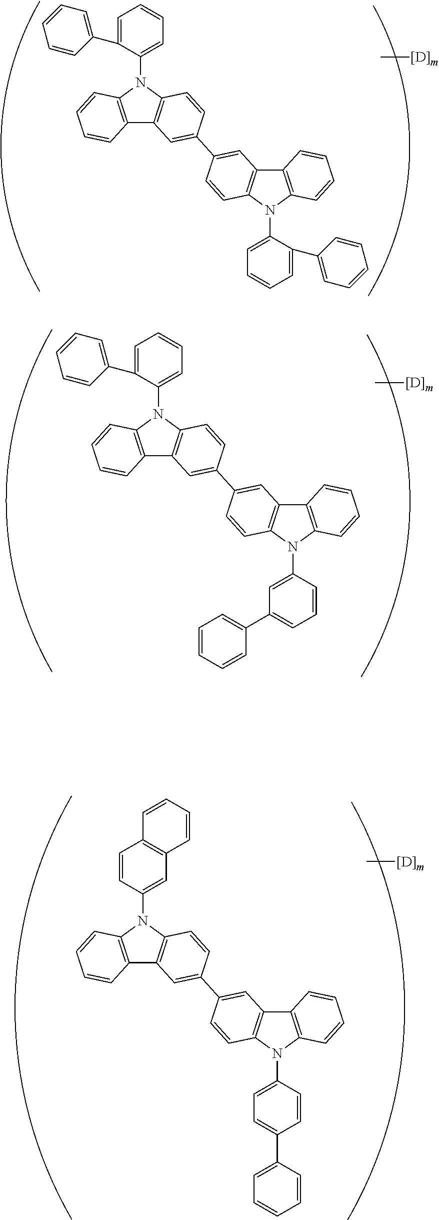

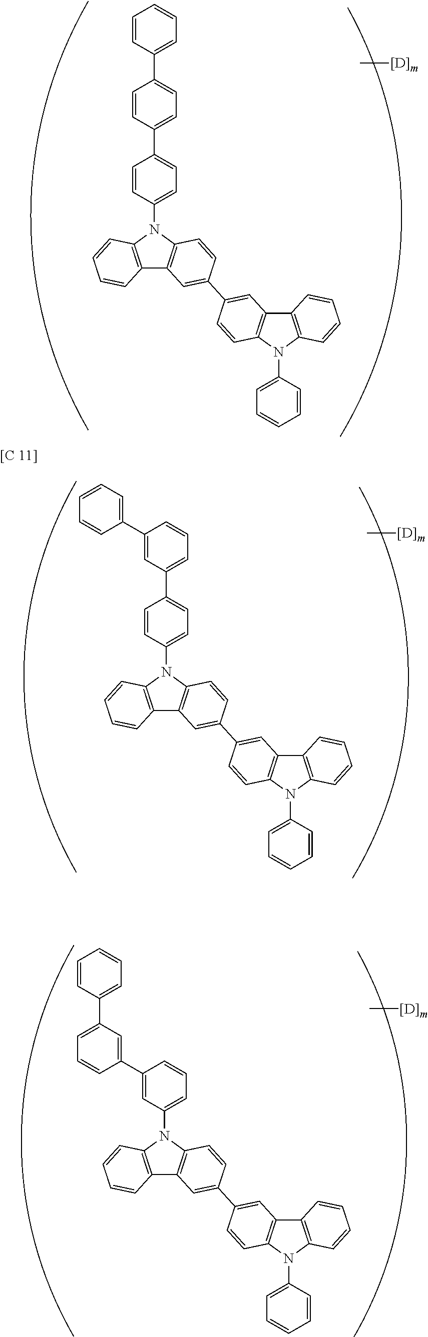

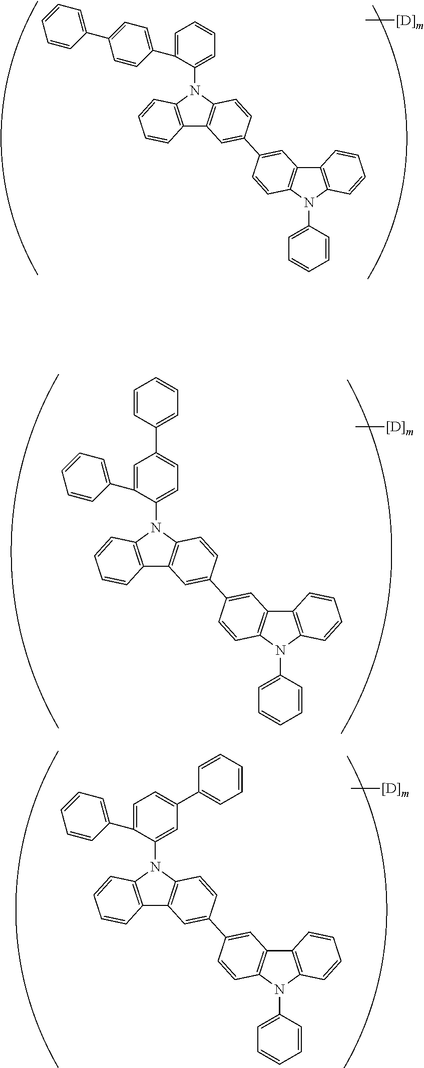

14. The mixed composition according to claim 6, wherein a difference in 50% weight reduction temperatures of the compound represented by the general formula (1) and the compound represented by the general formula (2) is within 20° C.

15. An organic electroluminescent device comprising a plurality of organic layers between an anode and a cathode, wherein at least one of the organic layers contains the mixed composition according to claim 2.

16. An organic electroluminescent device comprising a plurality of organic layers between an anode and a cathode, wherein at least one of the organic layers contains the mixed composition according to claim 3.

17. An organic electroluminescent device comprising a plurality of organic layers between an anode and a cathode, wherein at least one of the organic layers contains the mixed composition according to claim 4.

18. An organic electroluminescent device comprising a plurality of organic layers between an anode and a cathode, wherein at least one of the organic layers contains the mixed composition according to claim 4.

19. A method for producing an organic electroluminescent device comprising a plurality of organic layers including a light-emitting layer between an anode and a cathode, the method comprising the steps of: providing the mixed composition according to claim 6, and vapor-depositing the mixed composition from one vapor deposition source to form a light-emitting layer.
